(19) United States
(12) Patent Application Publication
(10) Pub. No.: US 2025/0283060 A1
(43) Pub. Date: Sep. 11, 2025

(54) COMPOSITIONS AND METHODS RELATING TO ENGINEERED RNA POLYMERASES WITH CAPPING ENZYMES

(71) Applicant: BOARD OF REGENTS, THE UNIVERSITY OF TEXAS SYSTEM, Austin, TX (US)

(72) Inventors: Andrew ELLINGTON, Austin, TX (US); Shaunak KAR, Austin, TX (US); Elizabeth GARDNER, Austin, TX (US); Jimmy GOLLIHAR, Houston, TX (US); Raghav SHROFF, Austin, TX (US)

(21) Appl. No.: 18/859,930

(22) PCT Filed: Apr. 25, 2023
(86) PCT No.: PCT/US2023/066168

§ 371 Date: Oct. 24, 2024

Related U.S. Application Data
(60) Provisional application No. 63/334,406, filed on Apr. 25, 2022.
(60) Provisional application No. 63/409,353, filed on Sep. 23, 2022.

(51) Int. Cl. C12N 9/12; C12N 15/81; C12Q 1/48

(52) U.S. Cl. CPC C12N 9/1247; C12N 15/81; C12Q 1/485; C07K 2319/00; C12Y 207/07006; C12Y 207/0705; G01N 2333/91255

(57) ABSTRACT

Disclosed herein is an engineered fusion enzyme composed of a prokaryotic phage RNA polymerase (T7 RNAP) and a viral capping enzyme (NP868R) for the generation of RNA transcripts containing a 5′ 7-methylguanylate cap.

### CROSS-REFERENCE TO RELATED APPLICATIONS

This application claims benefit of U.S. Provisional Application No. 63/334,406, filed Apr. 25, 2022, and U.S. Provisional Application No. 63/409,353, filed Sep. 23, 2022, both of which are hereby incorporated herein by reference in their entirety.

### BACKGROUND

The role of T7 RNA polymerase (RNAP) based transcription has been central to recombinant protein expression in prokaryotic chassis. Beyond such systems, the simplicity of T7 RNAP catalyzed transcription forms the corner stone for in vitro production of therapeutic RNAs as well as other biotechnological applications. However, the lack of the 5′ modifications in T7 RNAP derived transcripts has limited its use in protein expression in eukaryotic chassis organisms as well as generating functional eukaryotic mRNAs in vitro. Specifically, in the case of the latter, the transcripts can be modified separately using viral capping enzymes, suggesting that when both enzymes are used in conjunction (either fused or separately), the cooperative activity of the two enzymes can lead to the production of functional mRNAs in eukaryotes independent of the host transcriptional and capping machinery. The well characterized and commonly used capping enzyme obtained from vaccinia virus consists of two subunits. Recently, it was reported that the single subunit capping enzyme derived from African Swine Fever virus (NP868R) was able to catalyze all three reactions involved in the generation capped RNA. Thus, the use of this capping enzyme compared to vaccinia can greatly simplify in its implementation for mRNA/protein expression coupled to T7 RNAP.

Previously, the ability of wild type version of the fusion enzyme (NP868R fused to T7 RNAP via a flexible Glycine-serine linker) to generate capped transcripts was determined in mammalian cells (Jais 2019, Eaton 2017). In particular, the fusion enzyme was specifically used for the cytoplasmic expression of target genes under the control of T7 RNAP promoter and the levels of protein produced was used as a proxy for the efficiency of generation of capped transcripts. While the fusion of the capping enzyme augmented the protein expressed compared to T7 RNAP alone, the efficiency of capping was lower than that observed for Pol II derived transcripts (Jais 2019, Eaton 2017). Furthermore, this system has yet to be characterized in other eukaryotic chassis, and there has been no reported characterization of the enzyme for the nuclear expression of target genes in any chassis.

What is needed in the art is an engineered enzyme which combines RNA polymerase with capping capability, wherein the engineered enzyme has significantly increased polymerase and/or capping activity.

### SUMMARY

Disclosed herein is an engineered enzyme comprising a T7 RNA polymerase component and a capping enzyme component separated by a linker, wherein the T7 RNA polymerase component comprises 90% or more identity to SEQ ID NO: 3 and the capping enzyme component comprises 90% or more identity to SEQ ID NO: 5.

Also disclosed herein is an engineered enzyme comprising SEQ ID NO: 1 with at least one substitution which confers at least one improved property compared to SEQ ID NO: 1 without the substitution, and further wherein positions 881-896 of the engineered enzyme comprise a linker which can vary in length or amino acid composition.

Also disclosed is an engineered enzyme comprising an amino acid sequence with at least 90% identity to any one of SEQ ID NOS: 6-24.

Further disclosed are nucleic acids encoding the engineered enzymes, expression vectors comprising the nucleic acids, and host cells comprising the expression vectors.

Disclosed are methods of producing, as well as using, the engineered enzymes disclosed herein.

Also disclosed herein is a method of selecting one or more engineered enzymes comprising a non-eukaryotic polymerase component and a capping enzyme component, wherein the engineered enzyme comprises enhanced activity compared to a control, the method comprising: (a) creating nucleic acid encoding the one or more engineered enzyme variants, wherein said variants comprise a variant of a naturally occurring non-eukaryotic polymerase and a variant of a naturally occurring capping enzyme component; (b) integrating said nucleic acid encoding one or more engineered enzyme variants into a one or more eukaryotic cells, wherein said eukaryotic cells comprises a reporter, wherein said reporter is under the control of a polymerase promoter which is specific for the polymerase of the engineered enzyme, and further wherein the reporter is only expressed when it is capped by said capping enzyme; (c) expressing said nucleic acid encoding one or more engineered enzyme variants; and (d) determining which of the one or more variants confer enhanced activity compared to a control and selecting said engineered enzyme variant. An example of such a method is found in Example 1.

Also disclosed herein is a system which makes use of the method for directed evolution described above. Therefore, described herein is a system for selecting one or more engineered enzymes comprising a non-eukaryotic polymerase component and a capping enzyme component, wherein the engineered enzyme comprises enhanced activity, the system comprising a transformed eukaryotic cell, wherein said eukaryotic cell comprises a reporter plasmid, wherein said reporter plasmid is under the control of a polymerase promoter which is specific for the polymerase of the engineered enzyme, and further wherein the reporter is only expressed when it is capped by said capping enzyme. The eukaryotic cell can be designed for integration of one or more variant nucleic acids.

Further disclosed is a method of selecting one or more engineered enzymes comprising a non-eukaryotic polymerase component and a capping enzyme component, wherein the method comprises: a) providing nucleic acid encoding said engineered enzyme, wherein expression of the engineered enzyme is under control of a promoter, wherein said promoter is recognized by the non-eukaryotic polymerase of the engineered enzyme; b) placing the nucleic acid encoding the engineered enzyme under conditions suitable for its expression; and c) detecting mRNA produced by the engineered enzyme, and selecting said enzyme for further analysis.

Additional aspects and advantages of the disclosure will be set forth, in part, in the detailed description and any claims which follow, and in part will be derived from the detailed description or can be learned by practice of the various aspects of the disclosure. The advantages described below will be realized and attained by means of the elements and combinations particularly pointed out in the appended claims. It is to be understood that both the foregoing general description and the following detailed description are exemplary and explanatory only and are not restrictive of the disclosure.

### BRIEF DESCRIPTION OF THE FIGURES

The accompanying drawings, which are incorporated in and constitute a part of this specification, illustrate certain examples of the present disclosure and together with the description, serve to explain, without limitation, the principles of the disclosure. Like numbers represent the same elements throughout the figures.

FIG. 1 shows the design of the selection scheme for the evolution of the NP868R: T7 RNAP for the cotranscriptional capping and subsequent protein expression in Saccharomyces cerevisiae.

FIG. 2 shows the characterization of the evolved capping: T7 enzymes relative to wild-type (245) and catalytically dead enzyme (246).

FIG. 3 shows the 3D structure of T7 RNAP. T7 is capable of orthogonal transcription, has cross-species function, programmable promoter strength, and high levels of activity. However, it does not create capped transcripts for eukaryotic expression.

FIG. 4 is a bar chart showing bulk fluorescence measurements of capping-T7 polymerase variants expressing ZsGreen in yeast. The “broken” enzyme negative control consists of a K282A mutation which renders the capping domain inactive. “WT” indicates the capping-T7 fusion enzyme with an SV40 NLS but no additional mutations. V1, V2, and V3 correspond to ES-230, ES-368, and ES-443 respectively. ES-443 is 76-fold greater fluorescence than the broken control. ES-230 (V1) comes from round 17 of selection, but ES-368 (V2) and ES-443 (V3) both come from round 20. Fluorescence was measured in a Tecan M200 plate reader.

FIG. 5 shows single cell fluorescence of yeast populations containing the WT, V1, or V3 enzyme expressing the ZsGreen reporter. Fluorescence is measured as fluorescence intensity (height) in a Sony SA3800 spectral analyzer.

FIG. 6A-B shows all fusion variants of NPT7 were placed under the control of the galactose responsive promoter followed by the tENO2 terminator and integrated into the HO locus of the Saccharomyces cerevisiae BY4741 genome. The target gene (ZsGreen) was placed under the control of the T7 promoter, followed by the SV40 polyadenylation signal and the T7 terminator (A). This cloned into a 2-micron plasmid. Fold induction was calculated following the addition of 5% galactose relative to no induction (B).

FIG. 7A-B shows, for strains containing the fusion enzyme variants-WT, 433 and 443 and the target plasmid (A), the reporter expression was determined by adding different levels of the galactose to obtain the dose-response (B).

FIG. 8A-C shows, for comparing the activity of the variants, the same reporter gene (ZsGreen) under the control of the pGal promoter in two different contexts. First, it was integrated into the genome in the HO locus (IV target) (A) and the secondly, it was cloned into the same 2-micron plasmid (B). The galactose dose response for all constructs, and the fold induction was calculated based on the level of ZsGreen observed relative to the uninduced control (C).

FIG. 9A-D shows that the strength of T7 based expression can be controlled using mutant T7 promoters. 3 different mutant T7 promoters were picked which were predicted to give a panel of expression controlling the expression of ZsGreen (Panel B, Wild Type (WT) is SEQ ID NO: 25, Variant 2 (V2) is SEQ ID NO: 26, Variant 3 (V3) is SEQ ID NO: 27, and Variant 4 (V4) is SEQ ID NO: 28). All the mutant promoters were cloned in the same plasmid backbone (C, D).

FIG. 10A-C shows the promoter specificity of T7 RNAP can be obtained by introducing specific mutations in the DNA binding region of the gene. Specifically disclosed herein is that specificity of the fusion protein towards a panel orthogonal promoters can be similarly obtained by grafting the mutations into v443 (A, B). Each variant showed highly specific activity towards its own promoter and minimal cross-talk among the variants was observed (C). Panel C shows SEQ ID NOS: 25 (P_{T7}), SEQ ID NO: 29 (Pₒᵣₜₕₒ₁), SEQ ID NO: 30 (Pₒᵣₜₕₒ₂), SEQ ID NO: 31 (Pₒᵣₜₕₒ₃), SEQ ID NO: 32 (Pₒᵣₜₕₒ₄), and SEQ ID NO: 33 (Pₒᵣₜₕₒ₅).

FIG. 11 shows other reporter genes—BFP and mScarlet-I were cloned under the T7 promoter and transformed into strains containing v433 and v443. Upon induction with galactose, the expression of each gene was determined relative to the uninduced control.

FIG. 12A-C shows levels of the cargos—BFP and mScarlet-I (A) controlled using the set of mutant T7 promoters described previously. The relative order of strength of each promoter was conserved across the three reporter genes (B, C) thus conclusively demonstrating that the control of gene expression is exclusively controlled by the interaction of the fusion protein and its promoter.

FIG. 13A-D shows a plasmid encoding all three reporter genes-ZsGreen, mScarlet-I and BFP was cloned (A). Each gene was placed under the control of the T7 promoter (B). Wild Type (WT) is SEQ ID NO: 25. PT7 V2 (Variant 2) is SEQ ID NO: 26. PT7 V3 (Variant 3) is SEQ ID NO: 27. Versions of the same plasmid was built by placing ZsGreen under the control of mutant promoters (v2 and v3). These reporter plasmids were transformed into strains containing the fusion proteins-v433 and v443. Upon induction of the fusion protein with galactose, the relative expression of each gene to BFP was determined. As shown, the levels of Zsgreen can be predictably controlled while the expression of the other two genes remains consistent demonstrating multiplexed control of expression using the fusion proteins (C, D).

FIG. 14A-B shows a two-plasmid system for assaying the cytoplasmic activity of the fusion enzyme in mammalian cells. First, the NLS was removed from the WT NPT7 and v443 and placed under the control of the strong constitutive promoter-CMV. The reporter plasmid consisted of ZsGreen under the control of T7 promoter followed by a Kozak sequence. A synthetic sequence of a string 120 As were added followed by the T7 terminator (A). Upon transfection of both the plasmids in HEK293T, the levels of ZsGreen was determined after 48 hours post transfection. The variant 443 showed about 1.8-fold higher expression compared to WT NPT7 (B).

FIG. 15A-B shows the expression and purification of the fusion proteins. First, an affinity tagged (Twin Strep) of the T7 RNAP coding regions of the fusion protein was cloned (A). Following expression in BL21 cells and subsequent StrepTactin based purification, pure fractions of T7 RNAP were obtained and the yield was compared commercially available in vitro transcription mixes (Thermo and Promega) (B).

FIG. 16A-C shows that to assess the activity of the purified T7 RNAP variants including WT, the reporter plasmid was linearized and used as the transcription template (A). In vitro transcription was carried out using 200 ng template and 250 ug of purified T7 RNAP. The in vitro transcription reactions were carried out at two different temperatures (37 and 30), the RNA yield was analyzed using the Agilent TapeStation 4200 (B). Higher yields were obtained with Promega mix given that the higher amount of enzyme present (C).

FIG. 17A-C shows that for the expression and purification of the full-length fusion protein, an affinity tag was cloned under the control of the strong inducible E. coli promoter T5-lac (A). Following expression in BL21 cells and subsequent StrepTactin based purification, the elution fractions were analyzed using SDS-PAGE gel electrophoresis (B). The bands from the gel were excised and the full-length sequence was confirmed using mass spectrometry (C is full length sequence, SEQ ID NO: 34).

### DETAILED DESCRIPTION

### Definitions

Unless defined otherwise, all technical and scientific terms used herein generally have the same meaning as commonly understood by one of ordinary skill in the art to which tins invention pertains. Generally, the nomenclature used herein and the laboratory procedures of cell culture, molecular genetics, microbiology, biochemistry, organic chemistry, analytical chemistry and nucleic acid chemistry described below are those well-known and commonly employed in the art. Such techniques are well-known and described in numerous texts and reference works well known to those of skill in the art. Standard techniques, or modifications thereof, are used for chemical syntheses and chemical analyses. All patents, patent applications, articles and publications mentioned herein, both supra and infra, are hereby expressly incorporated herein by reference.

Although any suitable methods and materials similar or equivalent to those described herein find use in the practice of the present invention, some methods and materials are described herein. It is to be understood that this invention is not limited to the particular methodology, protocols, and reagents described, as these may vary, depending upon the context they are used by those of skill in the art. Accordingly, die terms defined immediately below are more fully described by reference to the application as a whole. All patents, patent applications, articles and publications mentioned herein, both supra and infra, are hereby expressly incorporated herein by reference.

Also, as used herein, the singular “a”, “an,” and “the” include the plural references, unless the context clearly indicates otherwise.

Numeric ranges are inclusive of the numbers defining the range. Thus, every numerical range disclosed herein is intended to encompass every narrower numerical range that falls within such broader numerical range, as if such narrower numerical ranges were all expressly written herein. It is also intended that every maximum (or minimum) numerical limitation disclosed herein includes every lower (or higher) numerical limitation, as if such lower (or higher) numerical limitations were expressly written herein.

The term “about” means an acceptable error for a particular value. In some instances “about” means within 0.05%, 0.5%, 1.0%, or 2.0%, of a given value range. In some instances, “about” means within 1, 2, 3, or 4 standard deviations of a given value.

Furthermore, the headings provided herein are not limitations of the various aspects or embodiments of the invention which can be had by reference to the application as a whole.

Accordingly, the terms defined immediately below are more fully defined by reference to the application as a whole. Nonetheless, in order to facilitate understanding of the invention, a number of terms are defined below.

Unless otherwise indicated, nucleic acids are written left to right in 5′ to 3′ orientation; ammo acid sequences are written left to right in amino to carboxy orientation, respectively.

As used herein, the term “comprising” and its cognates are used in their inclusive sense (i.e., equivalent to the term “including” and its corresponding cognates).

“EC” number refers to the Enzyme Nomenclature of the Nomenclature Committee of the International Union of Biochemistry and Molecular Biology (NC-IUBMB). The IUBMB biochemical classification is a numerical classification system for enzymes based on the chemical reactions they catalyze.

“ATCC” refers to the American Type Culture Collection whose biorepository collection includes genes and strains.

“NCBI” refers to National Center for Biological Information and the sequence databases provided therein.

As used herein, “T7 RNA polymerase” refers to a T7 bacteriophage-encoded DNA directed RNA polymerase that catalyzes the formation of RNA in the 5′ to 3′ direction.

As used herein, the term “cap” refers to the guanine nucleoside that is joined via its 5″ carbon to a triphosphate group that is, in turn, joined to the 5′ carbon of the most 5′ nucleotide of an mRNA transcript. In some embodiments, the nitrogen at the 7 position of guanine in the cap is methylated.

As used herein, the terms “capped RNA,” “5′ capped RNA,” and “capped mRNA” refer to RNA and mRNA, respectively that comprise the cap.

As used herein, “polynucleotide” and “nucleic acid’ refer to two or more nucleosides that are covalently linked together. The polynucleotide may be wholly comprised of ribonucleotides (i.e., NA), wholly comprised of deoxyribonucleotides (i.e., DNA), or comprised of mixtures of ribo- and deoxyribonucleotides. While the nucleosides will typically be linked together via standard phosphodiester linkages, the polynucleotides may include one or more non-standard linkages. The polynucleotide may be single-stranded or double-stranded, or may include both single-stranded regions and double-stranded regions. Moreover, while a polynucleotide will typically be composed of the naturally occurring encoding nucleobases (i.e., adenine, guanine, uracil, thymine and cytosine), it may include one or more modified and/or synthetic nucleobases, such as, for example, inosine, xanthine, hypoxanthine, etc. In some embodiments, such modified or synthetic nucleobases are nucleobases encoding amino acid sequences.

“Protein,” “polypeptide,” and “peptide” are used interchangeably herein to denote a polymer of at least two amino acids covalently linked by an amide bond, regardless of length or post-translational modification (e.g., glycosylation or phosphorylation).

“Amino acids” are referred to herein by either their commonly known three-letter symbols or by the one-letter symbols recommended by IUPAC-IUB Biochemical Nomenclature Commission. Nucleotides, likewise, may be referred to by their commonly accepted single letter codes. The abbreviations used for the genetically encoded amino acids are conventional and are as follows: alanine (Ala or A), arginine (Are or R), asparagine (Asn or N), aspartate (Asp or D), cysteine (Cys or C), glutamate (Glu or E), glutamine (Gin or Q), histidine (His or H), isoleucine (lie or I), leucine (Leu or L), lysine (Lys or K), methionine (Met or M), phenylalanine (Phe or F), proline (Pro or P), serine (Ser or S), threonine (Thr or T), tryptophan (Tip or W), tyrosine (Tyr or Y), and valine (Val or V). Θ054] When the three-letter abbreviations are used, unless specifically preceded by an “L” or a “D” or clear from the context in which the abbreviation is used, the amino acid may be in either the L- or D-configuration about a-carbon (C«). For example, whereas “Ala” designates alanine without specifying the configuration about the α-carbon, “D-Ala” and “L-A3a” designate D-alanine and L-alanine, respectively. When the one-letter abbreviations are used, upper case letters designate ammo acids in the L-configuration about the a-carbon and lower case letters designate amino acids in the D-configuration about the a-carbon. For example, “A” designates L-alanine and “a” designates D-alanine. When polypeptide sequences are presented as a string of one-letter or three-letter abbreviations (or mixtures thereof), the sequences are presented in the amino (N) to carboxy (C) direction in accordance with common convention.

The abbreviations used for the genetically encoding nucleosides are conventional and are as follows: adenosine (A); guanosine (G): cytidine (C); thymidine (T); and uridine (U). Unless specifically delineated, the abbreviated nucleosides may be either ribonucleosides or deoxyribonucleosides. The nucleosides may be specified as being either ribonucleosides or deoxyribonucleosides on an individual basis or on an aggregate basis. When nucleic acid sequences are presented as a string of one-letter abbreviations, the sequences are presented in the 5′ to 3′ direction in accordance with common convention, and the phosphates are not indicated.

The term “engineered,” “recombinant,” “non-naturally occurring,” and “‘variant,” when used with reference to a cell, a polynucleotide or a polypeptide refers to a material or a material corresponding to the natural or native form of the material that has been modified in a manner that would not otherwise exist in nature or is identical thereto but produced or derived from synthetic materials and/or by manipulation using recombinant techniques.

As used herein, “wild-type” and “naturally-occurring” refer to the form found in nature. For example a wild-type polypeptide or polynucleotide sequence is a sequence present in an organism that can be isolated from a source in nature and which has not been intentionally modified by human manipulation. In the present disclosure, “wild type” also refers to the fusion of wild-type RNAP with a wild-type capping enzyme from another organism. What is meant is that the fusion protein has not been further mutated, although it doesn't exist in nature because it is a fusion of enzymes from two different organisms. For example, a “wild type” fusion protein is found in SEQ ID NO: 1.

“Coding sequence” refers to that part of a nucleic acid (e.g., a gene) that encodes an amino acid sequence of a protein.

The term “percent (%) sequence identity” is used herein to refer to comparisons among polynucleotides and polypeptides, and are determined by comparing two optimally aligned sequences over a comparison window, wherein the portion of the polynucleotide or polypeptide sequence in the comparison window may comprise additions or deletions {i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman (Smith and Waterman, Adv. Appl. Math., 2:482) [1981], by the homology alignment algorithm of Needleman and Wunsch (Needleman and Wunsch, J. Mol. Biol, 48:443 [1970), by the search for similarity method of Pearson and Lipman (Pearson and Lipman, Proc. Natl. Acad. Sci. USA 85:2444 [1988]), by computerized implementations of these algorithms (e.g., GAP, BESTFIT, FASTA, and TFASTA in the GCG Wisconsin Software Package), or by visual inspection, as known in the art. Examples of algorithms that are suitable for determining percent sequence identity and sequence similarity include, but are not limited to the BLAST and BLAST 2.0 algorithms, which are described by Altschul et al. (See, Altschul et al., J. Mol. Biol, 215:403-410 [1990]; and Altschul et al., Nucleic Acids Res, 25:3389-3402 [1977], respectively). Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (See, Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum, achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (See, Henikoff and Henikoff, Proc. Natl. Acad. Sci. USA 89:10915 [1989]). Exemplary determination of sequence alignment and % sequence identity can employ the BESTFIT or GAP programs in the GCG Wisconsin Software package (Accelrys, Madison WI), using default parameters provided.

“Reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, at least 100 residues in length or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically-performed by comparing sequences of the two polynucleotides or polypeptides over a “comparison window” to identify and compare local regions of sequence similarity. In some embodiments, a “reference sequence” can be based on a primary amino acid sequence, where the reference sequence is a sequence that can have one or more changes in the primary sequence.

“Comparison window” refers to a conceptual segment of at least about 20 contiguous nucleotide positions or amino acids residues wherein a sequence may be compared to a reference sequence of at least 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. The comparison window can he longer than 20 contiguous residues, and includes, optionally 30, 40, 50, 100, or longer windows.

“Corresponding to”, “reference to” or “relative to” when used in the context of the numbering of a given amino acid or polynucleotide sequence refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered T7 RNA polymerase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Amino acid difference” or “residue difference” refers to a difference in the amino acid residue at a position of a polypeptide sequence relative to the amino acid residue at a corresponding position in a reference sequence. The positions of amino acid differences generally are referred to herein as “Xn,” where n refers to the corresponding position in the reference sequence upon which the residue difference is based. For example, a “residue difference at position K9 as compared to SEQ ID NO: 1” refers to a difference of the amino acid residue at the polypeptide position corresponding to position 9 of SEQ ID NO: 1. Thus, if the reference polypeptide of SEQ ID NO: 1 has a lysine at position 9, then a “residue difference at position K9 as compared to SEQ ID NO: 1” an amino acid substitution of any residue other than lysine at the position of the polypeptide corresponding to position 9 of SEQ ID NO: 1. In most instances herein, the specific ammo acid residue difference at a position is indicated as “XnY” where “Xn” specified the corresponding position as described above, and “Y” is the single letter identifier of the amino acid found in the engineered polypeptide (i.e., the different residue than in the reference polypeptide). In some instances (e.g., in the Tables provided in the Examples herein), the present disclosure also provides specific amino acid differences denoted by the conventional notation “AnB”, where A is the single letter identifier of the residue in the reference sequence, “n” is the number of the residue position in the reference sequence, and B is the single letter identifier of the residue substitution in the sequence of the engineered polypeptide. Referring again to the example given above, a substitution for asparagine in place of lysine at position K would read, “K9N.” In some instances, a polypeptide of the present disclosure can include one or more amino acid residue differences relative to a reference sequence, which is indicated by a list of the specified positions where residue differences are present relative to the reference sequence. In some embodiments, the enzyme variants comprise more than one substitution. These substitutions are separated by a slash for ease in reading (e.g., R10K/R10I). The present application includes engineered polypeptide sequences comprising one or more ammo acid differences that include either/or both conservative and non-conservative amino acid substitutions.

“Conservative amino acid substitution” refers to a substitution of a residue with a different residue having a similar side chain, and thus typically involves substitution of the amino acid in the polypeptide with amino acids within the same or similar defined class of amino acids. By way of example and not limitation, an amino acid with an aliphatic side chain may be substituted with another aliphatic amino acid (e.g., alanine, valine, leucine, and isoleucine); an ammo acid with hydroxy! side chain is substituted with another amino acid with a hydroxy! side chain (e.g., serine and threonine); an amino acids having aromatic side chains is substituted with another amino acid having an aromatic side chain (e.g., phenylalanine, tyrosine, tryptophan, and histidine): an amino acid with a basic side chain is substituted with another ammo acid with a basis side chain (e.g., lysine and arginine); an amino acid with an acidic side chain is substituted with another amino acid with an acidic side chain (e.g., aspartic acid or glutamic acid); and/or a hydrophobic or hydrophilic ammo acid is replaced with another hydrophobic or hydrophilic amino acid, respectively.

“Non-conservative substitution” refers to substitution of an amino acid in the polypeptide with an amino acid with significantly differing side chain properties. Non-conservative substitutions may use amino acids between, rather than within, the defined groups and affects (a) the structure of the peptide backbone in the area of the substitution (e.g., proline for glycine) (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

“Deletion” refers to modification to the polypeptide by removal of one or more amino acids from the reference polypeptide. Deletions can comprise removal of 1 or more amino acids, 2 or more ammo acids, 5 or more amino acids, 10 or more ammo acids, 15 or more ammo acids, or 20 or more amino acids, up to 10% of the total number of amino acids, or up to 20% of the total number of amino acids making up the reference enzyme while retaining enzymatic activity and/or retaining the improved properties of an engineered enzyme. Deletions can be directed to the internal portions and/or terminal portions of the polypeptide. In various embodiments, the deletion can comprise a continuous segment or can be discontinuous.

“Insertion” refers to modification to the polypeptide by addition of one or more ammo acids from the reference polypeptide, insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

“Isolated polypeptide” refers to a polypeptide which is substantially separated from other contaminants that naturally accompany it (e.g., protein, lipids, and polynucleotides). The term embraces polypeptides which have been removed or purified from their naturally-occurring environment or expression system (e.g., host cell or in vitro synthesis). The recombinant T7 RNA polymerase polypeptides may be present within a cell, present in the cellular medium, or prepared in various forms, such as lysates or isolated preparations. As such, in some embodiments, the recombinant T7 RNA polymerase polypeptides can be an isolated polypeptide.”

“Substantially pure polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure T7 RNA polymerase composition comprises about 60% or more, about 70% or more, about 80% or more, about 90% or more, about 95% or more, and about 98% or more of all macromolecular species by mole or % weight present in the composition, in some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species, in some embodiments, the isolated recombinant T7 RNA polymerase polypeptides are substantially pure polypeptide compositions.

“Improved enzyme property” of a T7 RNA polymerase and/or capping enzyme refers to an engineered T7 RNA polymerase polypeptide and/or capping enzyme that exhibits an improvement in any enzyme property as compared to a reference T7 RNA polymerase polypeptide and/or a wild-type T7 RNA polymerase polypeptide and/or another engineered T7 RNA polymerase polypeptide, or to a reference capping enzyme and/or a wild-type capping enzyme and/or another engineered capping enzyme. Improved properties include, but are not limited to such properties which relate to improved capping enzyme specific properties, improved T7 RNA polymerase properties, and improved properties resulting from both enzymes together. These include increased selectivity for cap analog over GTP, increased fidelity of replication, increased RNA yield, increased protein expression, increased thermoactivity, increased pseudouridine incorporation or other RNA base analogs, increased thermostability, increased pH activity, increased stability, increased enzymatic activity, increased substrate specificity or affinity, increased specific activity, increased resistance to substrate or end-product inhibition (including pyrophosphate), increased chemical stability, improved solvent stability, increased tolerance to acidic or basic pH, increased tolerance to proteolytic activity (i.e., reduced sensitivity to proteolysis), reduced aggregation, increased solubility, and altered temperature profile. Improved capping properties include increased RNA triphosphatase activity, guanylyltransferase activity, and methyltransferase activity, for example. Also included are increased chromatin remodeling or epigenetic modification in eukaryotic cells. Another improvement is less abortive transcripts using the T7 variants compared to wild type. Improved joint properties can also include altered T7 kinetics relating to initiation and elongation that can enhance capping efficiency.

“Increased enzymatic activity” or “enhanced catalytic activity” refers to an improved property of the engineered T7 RNA polymerase, which can be represented by an increase in specific activity (e.g., product produced/time/weight protein) or an increase in percent conversion of the substrate to the product (e.g., percent conversion of starting amount of substrate to product in a specified time period using a specified amount of variant T7 RNA polymerase as compared to the reference T7 RNA polymerase. Exemplary methods to determine enzyme activity are provided in the Examples. Any property relating to enzyme activity may be affected.

“Hybridization stringency” relates to hybridization conditions, such as washing conditions, in the hybridization of nucleic acids. Generally, hybridization reactions are performed under conditions of lower stringency, followed by washes of varying but higher stringency. The term “moderately stringent hybridization” refers to conditions that perm it target-DNA to bind a complementary nucleic acid that has about 60% identity, preferably about 75% identity, about 85% identity to the target DNA, with greater than about 90% identity to target-polynucleotide. Exemplary moderately stringent conditions are conditions equivalent to hybridization in 50% formamide, 5×Denhart's solution, 5×SSPE, 0.2% SDS at 42° C., followed by washing in 0.2×SSPE, 0.2% SDS, at 42° C. “High stringency hybridization” refers generally to conditions that are about 10° C. or less from the thermal melting temperature Tm as determined under the solution condition for a defined polynucleotide sequence. In some embodiments, a high stringency condition refers to conditions that permit hybridization of only those nucleic acid sequences that form stable hybrids in 0.018M NaCl at 65° C. (i.e., if a hybrid is not stable in 0.018M NaCl at 65° C., it will not be stable under high stringency conditions, as contemplated herein). High stringency conditions can be provided, for example, by hybridization in conditions equivalent to 50% formamide, 5*Denhart's solution, 5*SSPE, 0.2% SDS at 42° C., followed by washing in 0.1×SSPE, and 0.1% SDS at 65° C. Another high stringency condition is hybridizing in conditions equivalent to hybridizing in 5×SSC containing 0.1% (w:v) SDS at 65° C. and washing in 0.1×SSC containing 0.1% SDS at 65° C. Other high stringency hybridization conditions, as well as moderately stringent conditions, are described in the references cited above.

“Codon optimized” refers to changes in the codons of the polynucleotide encoding a protein to those preferentially used in a particular organism such that the encoded protein is more efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the T7 RNA polymerase enzymes may be codon optimized for optimal production from the host organism selected for expression.

“Control sequence” refers herein to include all components, which are necessary or advantageous for the expression of a polynucleotide and/or polypeptide of the present application. Each control sequence may be native or foreign to the nucleic acid sequence encoding the polypeptide. Such control sequences include, but are not limited to, a leader, polyadenylation sequence, propeptide sequence, promoter sequence, signal peptide sequence, initiation sequence and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleic acid sequence encoding a polypeptide.

“Operably linked” is defined herein as a configuration in which a control sequence is appropriately placed (i.e., in a functional relationship) at a position relative to a polynucleotide of interest such that the control sequence directs or regulates the expression of the polynucleotide and/or polypeptide of interest.

“Promoter sequence” refers to a nucleic acid sequence that is recognized by a host cell for expression of a polynucleotide of interest, such as a coding sequence. The promoter sequence contains transcriptional control sequences, which mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

“Suitable reaction conditions” refers to those conditions in the enzymatic conversion reaction solution (e.g., ranges of enzyme loading, substrate loading, temperature, pH, buffers, co-solvents, etc.) under which a 17 RNA polymerase polypeptide of the present application is capable of converting a substrate to the desired product compound.

“Substrate” in the context of an enzymatic conversion reaction process refers to the compound or molecule acted on by the T7 RNA polymerase polypeptide.

“Product” in the context of an enzymatic conversion process refers to the compound or molecule resulting from the action of the T7 RNA polymerase polypeptide on a substrate.

As used herein the term “culturing” refers to the growing of a population of microbial cells under any suitable conditions (e.g., using a liquid, gel or solid medium).

Recombinant polypeptides can be produced using any suitable methods known the art. Genes encoding the wild-type polypeptide of interest can be cloned in vectors, such as plasmids, and expressed in desired hosts, such as E. coli, S. cerevisiae, etc. Variants of recombinant polypeptides can be generated by various methods known in the art. Indeed, there is a wide variety of different mutagenesis techniques well known to those skilled in the art. In addition, mutagenesis kits are also available from many commercial molecular biology suppliers. Methods are available to make specific substitutions at defined amino acids (site-directed), specific or random mutations in a localized region of the gene (regio-specific), or random mutagenesis over the entire gene (e.g., saturation mutagenesis). Numerous suitable methods are known to those in the art to generate enzyme variants, including but not limited to site-directed mutagenesis of single-stranded DNA or double-stranded DNA using PCR, cassette mutagenesis, gene synthesis, error-prone PCR, shuffling, and chemical saturation mutagenesis, or any other suitable method known in the art. Non-limiting examples of methods used for DNA and protein engineering are provided in the following patents: U.S. Pat. Nos. 6,117,679; 6,420,175; 6,376,246; 6,586,182; 7,747,391; 7,747,393; 7,783,428; and 8,383,346. After the variants are produced, they can he screened for any desired property (e.g., high or increased activity, or low or reduced activity, increased thermal activity, increased thermal stability, and/or acidic pH stability, etc.).

In some embodiments, “recombinant T7 RNA polymerase polypeptides” (also referred to herein as “engineered T7 RNA polymerase polypeptides,” “variant T7 RNA polymerase enzymes,” and “T7 RNA polymerase variants”) find use.

As used herein, a “vector” is a DNA construct for introducing a DNA sequence into a cell. In some embodiments, the vector is an expression vector that is operably linked to a suitable control sequence capable of affecting the expression in a suitable host of the polypeptide encoded in the DNA sequence. In some embodiments, an “expression vector” has a promoter sequence operably linked to the DNA sequence (e.g., transgene) to drive expression in a host ceil, and in some embodiments, also comprises a transcription terminator sequence.

As used herein, the term “expression” includes any step involved in the production of the polypeptide including, but not limited to, transcription, post-transcriptional modification, translation, and post-translational modification. In some embodiments, the term also encompasses secretion of the polypeptide from a cell.

As used herein, the term “produces” refers to the production of proteins and/or other compounds by cells. It is intended that the term encompass any step involved in the production of polypeptides including, but not limited to, transcription, post-transcriptional modification, translation, and post-translational modification. In some embodiments, the term also encompasses secretion of the polypeptide from a cell.

As used herein, an amino acid or nucleotide sequence (e.g., a promoter sequence, signal peptide, terminator sequence, etc.) is “heterologous” to another sequence with which it is operably linked if the two sequences are not associated in nature.

As used herein, the terms “host cell” and “host strain” refer to suitable hosts for expression vectors comprising DNA provided herein (e.g., the polynucleotides encoding the T7 RNA polymerase variants). In some embodiments, the host cells are prokaryotic or eukaryotic cells that have been transformed or transfected with vectors constructed using recombinant DNA techniques as known in the art.

The term “analog” when used in reference to a polypeptide, means a polypeptide having more than 70% sequence identity but less than 100% sequence identity (e.g., more than 75%, 78%, 80%, 83%, 85%, 88%, 90%, 91%, 92%, 93%, 94%, 95%, 96%, 97%, 98%, 99% sequence identity) with a reference polypeptide. In some embodiments, analogues means polypeptides that contain one or more non-naturally occurring ammo acid residues including, but not limited, to homoarginine, ornithine and norvaline, as well as naturally occurring amino acids. In some embodiments, analogues also include one or more D-amino acid residues and non-peptide linkages between two or more amino acid residues.

The term “effective amount” means an amount sufficient to produce the desired result. One of general skill in the art may determine what the effective amount by using routine experimentation.

The terms “isolated” and “purified” are used to refer to a molecule (e.g., an isolated nucleic acid, polypeptide, etc.) or other component that is removed from at least one other component with which it is naturally associated. The term “purified” does not require absolute purity, rather it is intended as a relative definition.

As used herein, “composition” and “formulation” encompass products comprising at least one engineered T7 RNA polymerase of the present invention, intended for any suitable use (e.g., research, diagnostics, etc.).

The term “transcription” is used to refer to the process whereby a portion of a DNA template is copied into RNA by the action of an RNA polymerase enzyme.

The term “DNA template” is used to refer to a double or single-stranded DNA molecule including a promoter sequence and a sequence coding for the RNA product of transcription.

The term “promoter” is used to refer to a DNA sequence that is recognized by RNA polymerase as the start site of transcription. The promoter recruits RNA polymerase, and in the case of T7 RNA polymerase, determines the start site of transcription.

The term “RNA polymerase” is used to refer to a DNA-directed RNA polymerase, which copies a DNA template into an RNA polynucleotide, by incorporating nucleotide triphosphates stepwise into the growing RNA polymer.

The terms “messenger RNA” and “mRNA” are used to refer to RNA molecules that code for a protein. This protein is decoded through the action of translation.

The terms “7-methylguanosine cap,” “7meG,” “five-prime cap,” and “5″cap” are used in reference to a specific modified nucleotide structure present at the 5′ end of eukaryotic mRNAs. The 7-methylguanosine cap structure is attached through a 5′ to 5′ triphosphate linkage to the first nucleotide in the mRNA. In vivo, this cap structure is added to the 5′ end of a nascent mRNA through the successive activities of multiple enzymes. In vitro, the cap can be incorporated directly at the initiation of transcription by an RNA polymerase by use of a cap analog.

The term “cap analog” refers to a dinucleotide containing a 5 ‘-5’ di-, tri-, or tetra-phosphate linkage. One end of the dinucleotide terminates in either a guanosine or substituted guanosine residue; it is this end from which RNA polymerase will initiate transcription by extending from the 3′ hydroxyl. The other end of the dinucleotide is a guanosine that mimics the eukaryotic cap structure, and will typically have 7-methyl-, 7-benzyl-, or 7-ethyl-substitutions and/or 7-aminomethyl or 7-aminoethyl substitutions. In some cases, this nucleotide also is substituted at the 3′ hydroxyl group to prevent initiation of transcription from the cap end of the molecule.

The terms “ARCA” and “anti-reverse cap analog” refer to chemically modified forms of cap analogs, designed to maximize the efficient of in vitro translation by ensuring that the cap analog is properly incorporated into the transcript in the correct orientation. These analogs find use in enhancing translation. In some embodiments, the ARCAs known in the art find use (e.g., Peng et al., Org. Lett., 4:161-164 [2002]).

As used herein the term “endogenous DNA-dependent RNA polymerase” relates to the endogenous DNA-dependent RNA polymerase of said host cell. When the host cell is a eukaryotic cell, said endogenous DNA-dependent RNA polymerase is the RNA polymerase II.

As used herein the term “endogenous capping enzyme” refers to the endogenous capping enzyme of said host cell.

As used herein the term “inhibiting the expression of a protein” relates to a decrease of at least 20%, particularly at least 35%, at least 50% and more particularly at least 65%, at least 80%, at least 90% of expression of said protein. Inhibition of protein expression can be determined by techniques well known to one skilled in the art, including but not limiting to Northern-Blot, Western-Blot, RT-PCR.

The term “riboswitch” is used to refer to an autocatalytic RNA enzyme that cleaves itself or another RNA in the presence of a ligand.

The term “fidelity” is used to refer to the accuracy of an RNA polymerase in transcribing, or copying, a DNA template into an RNA polynucleotide. Inaccurate transcription can result in single-nucleotide polymorphisms (SNPs) or Indels.

The terms “single-nucleotide polymorphism” or “SNP” refer to a change in the nucleotide present at a single position in polynucleotide. In the context of transcription, SNPs can result from misincorporation of a non-complementary ribonucleotide (A, C, G, or U) by RNA polymerase at a position on the DNA template.

The term “Indel” is used to refer to an insertion or deletion of one or more polynucleotides. In the context of transcription by RNA polymerase, indel errors can result from the addition of a one or more extra ribonucleotides or failure to incorporate one or more nucleotides at a position on the DNA template.

The term “selectivity” is used to refer to the trait of an enzyme to have higher activity against one substrate as compared to another substrate during a catalyzed reaction. In the context of co-transcriptional capping, the RNA polymerase may have high or low selectivity for a cap analog over GTP.

‘The term “inorganic pyrophosphatase” is used to refer to an enzyme that degrades inorganic pyrophosphate to orthophosphate.

### General Description

Prior to this invention, the state of the art for orthogonal protein expression in eukaryotes relied on the use of synthetic transcription factors in conjunction with characterized endogenous or viral promoters. However, the entire process was still reliant on the host RNA polymerase II and its regulatory associated limitations. The use of T7 RNAP for transcription and a viral capping enzyme (NP868R) for the expression of target genes completely decouples the process from Pol II, thus providing an orthogonal mode of control and potentially allowing the overexpression of a target gene. The use of a completely orthogonal RNA polymerase for the expression of functional mRNAs greatly expands the expression and control capabilities and expand the repertoire of synthetic circuit elements. This engineered enzyme has been evolved to function optimally not only in a yeast background, but also in other eukaryotic hosts.

This orthogonal mode of gene expression is highly desirable for protein overexpression because it proceeds without inhibitory feedback which is characteristic of cell stress. Orthogonal gene expression also has use in cellular circuits that are used outside of the laboratory since environmental stresses can otherwise affect gene expression.

Perhaps more significantly, beyond in vivo applications, this fusion enzyme also streamlines the process of generating capped mRNA in vitro in a single reaction. Normally this workflow proceeds with two distinct steps: T7-driven RNA transcription followed by capping with a viral capping enzyme (typically vaccinia virus capping enzyme). This invention allows for fewer required reagents and simplifies the reaction to a single step. In addition, the mutations observed solely in the T7 RNAP domain of the evolved variants can result in higher processivity, and lesser abortive products compared to that with wild type enzyme. In the case of NP868R, the evolved versions can result in improved capping efficiencies compared to the industrial workhorse-vaccinia capping enzyme.

Further, this invention instantiates methods for engineering capping enzymes for mRNA vaccine manufacturing. A current bottleneck in mRNA vaccines is the production and activity of the capping enzyme. Methods described herein greatly aid in the generation of capping enzyme variants for scale up manufacturing of mRNA for vaccines and therapeutics.

Disclosed herein is an engineered enzyme comprising a T7 RNA polymerase (SEQ ID NO: 3) and a single subunit capping enzyme derived from African Swine Fever virus (NP8968R) (SEQ ID NO: 5). These two components can be linked by a linker. Such linkers are known to those of skill in the art, and can be 5, 6, 7, 8, 9, 10, 11, 12, 13, 14, 15, 16, 17, 18, 19, 20, 21, 22, 23, 24, 25, 26, 27, 28, 29, or 30 amino acid residues in length (or longer or shorter, as one of skill in the art can determine). The linker can vary in content as well as length. An example of a linker can be found in SEQ ID NO: 4. The engineered enzyme can also comprise a signal peptide, such as a nuclear localization signal (NLS). An example of a nuclear localization sequence (NLS) can be found in SEQ ID NO: 2, as well as in positions 1-13 of SEQ ID NO: 1. Mutations in the NLS can lead to increased activity of the engineered enzyme.

Also disclosed are variants of any of SEQ ID NOS: 2-6 wherein said variants comprise 1, 2, 3, 4, 5, 6, 7, 8, 9, 10, 11, 12, 13, 14, 15, 16, 17, 18, 19, or 20 or more amino acid variations, wherein said variations comprise deletions, insertions, or substitutions. These variations can confer improved properties, which are discussed in detail below. Put another way, disclosed herein are variants of any of SEQ ID NOS: 2-6 which have 80, 81, 82, 83, 84, 85, 86, 87, 88, 89, 90, 91, 92, 93, 94, 95, 96, 97, 98, or 99% identity to SEQ ID NOS: 2-6, respectively.

When the components described above are combined, the result is SEQ ID NO: 1, which comprises the NLS, the T7 RNAP, a linker, and the capping enzyme. Variants of SEQ ID NO: 1 can have 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 or more differences in amino acid composition compared with SEQ ID NO: 1. Some of these mutations are typified in SEQ ID NOS: 6-24. Positions 881-896 of SEQ ID NO: 1 can encompass the linker. As discussed above, the linker can be varied and the enzyme can still retain its function. When referring to percent identity to SEQ ID NO: 1, the linker may or may not be considered. For example, a variant of SEQ ID NO: 1 can have 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% to SEQ ID NO: 1. This percentage can include, or not include, the linker.

Mutations to the T7 RNAP, the capping enzyme, and/or the NLS were found to confer surprising and unexpected benefits to the activity of the engineered enzyme. For example, the enzyme can have higher protein expression compared to wild type enzyme. In other examples, the improved property can be selected from improved selectivity for capping, improved processivity of capping, improved protein expression, improved RNA yield, improved stability in storage buffer, improved stability under reaction conditions, improved processivity of translation, improved thermostability, and improved transcription fidelity. The improved property can also include improved capping of enzymatic activities, such as improvement to activity of RNA triphosphatase guanyltransferase, and/or methyltransferase.

By “improved” is meant that the properties specified above are improved compared to the wild-type T7 RNAP, capping enzyme, NLS, linker, or combination of all of these, by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or by 2, 3, 4, 5, 6, 7, 8, 9, 10, 11, 12, 13, 14, 15, 16, 17, 18, 19, or 20 fold or more.

As discussed above, the sequences can comprise mutations which include substitutions, deletions, or insertions. Substitutions include, but are not limited to, those found in Table 1 and Table 2. One or more of these substitutions can occur in the same engineered enzyme. Examples of engineered enzymes comprising these substitutions can be found in SEQ ID NOS: 6-24.

Further disclosed herein is a capping enzyme derived from African Swine Fever virus (NP868R), wherein said capping enzyme comprises one or more mutations which confer improved properties to the enzyme. These improved properties are disclosed elsewhere herein. The capping enzyme can comprise 90, 91, 92, 93, 94, 95, 96, 79, 98, or 99% or more identity to SEQ ID NO: 5. Put another way, the capping enzyme can comprise 1, 2, 3, 4, 5, 6, 7, 8, 9, 10, 11, 12, 13, 14, 15, 16, 17, 18, 19, or 20 or more amino acid deletions, insertions, or substitutions which confer improved properties.

Also disclosed is a T7 RNA polymerase, wherein said T7 RNA polymerase comprises one or more mutations which confer improved properties of the enzyme. These improved properties are disclosed elsewhere herein. The T7 RNA polymerase can comprise 90, 91, 92, 93, 94, 95, 96, 79, 98, or 99% or more identity to SEQ ID NO: 3. Put another way, the T7 RNA polymerase can comprise 1, 2, 3, 4, 5, 6, 7, 8, 9, 10, 11, 12, 13, 14, 15, 16, 17, 18, 19, or 20 or more amino acid deletions, insertions, or substitutions which confer improved properties.

Also disclosed herein is a nucleic acid encoding the engineered enzymes of this invention. Said group of isolated nucleic molecules encoding the engineered enzyme according to the invention can comprise all of the nucleic acid molecules which are necessary and sufficient to obtain an engineered enzyme according to the invention by their expression. The nucleic acid encoding the engineered enzyme can be operably linked to a control sequence.

In particular, the nucleic acid molecule according to the invention can be operatively linked to a promoter. The link of the nucleic acid to a promoter for a eukaryotic DNA-dependent RNA polymerase, preferably for RNA polymerase II, has notably the advantage that when the chimeric enzyme of the invention is expressed in an eukaryotic host cell, the expression of the chimeric enzymes is driven by the eukaryotic RNA polymerase, preferably the RNA polymerase II. These chimeric enzymes, in turn, can initiate transcription of the transgene. If tissue-specific RNA polymerase II promoters are used, the chimeric enzyme of the invention can be selectively expressed in the targeted tissues/cells. Said promoter can be a constitutive promoter or an inducible promoter well known by one skilled in the art. The promoter can be developmentally regulated, inducible or tissue specific.

The invention also relates to a vector comprising a nucleic acid molecule according to the invention. Said vector can be appropriated for semi-stable or stable expression. The invention also relates to a group of vectors comprising said group of isolated nucleic acid molecules according to the invention. Particularly, said vector according to the invention is a cloning or an expression vector.

The invention also relates to a host cell comprising a nucleic acid molecule according to the invention or a vector according to the invention or a group of vectors according to the invention. The host cell according to the invention can be useful for large-scale protein production.

The invention also relates to a genetically engineered eukaryotic organism, which expresses an engineered enzyme encoded by the nucleic acid molecule or the group of isolated nucleic acid molecules according to the invention, in particular an engineered enzyme according to the invention. Said eukaryotic organism can be any single-celled eukaryotic organisms like yeast. Also contemplated are organisms such as mammals, or any other animals or plants. Examples in yeast include, but are not limited to, Saccharomyces cerevisiae and Pichia pastoris. Examples of mammalian cells which can be used with the invention include, but are not limited to, HEK 293, Jurkat, CHO, COS, and primary human cells including immune cells and stem cells. This invention can be used in vivo or in vitro.

The invention also relates to the use of the engineered enzymes according to the invention, for the production of RNA molecule with 5′-terminal cap. Particularly, said RNA molecule can be synthesized by a bacteriophage DNA-dependent RNA polymerase, such as T7 RNAP.

The invention also relates to the use of the engineered enzymes according to the invention or an isolated nucleic acid molecule or a group of isolated nucleic acid molecules according to the invention, for the production of protein, in particular protein of therapeutic interest like a vaccine or an antibody, particularly in eukaryotic systems, such as in vitro synthesized protein assay or cultured cells. These can be used in the context of purified protein therapeutics or as cell factories where the protein is continuously produced in or outside of the host organism.

The invention also relates to method for producing an RNA molecule with a 5′-terminal cap, said method comprising the step of expressing in the host cell a nucleic acid molecule or a group of isolated nucleic acid molecules according to the invention, wherein said DNA sequence is covalently linked to at least one sequence encoding the RNA element of said protein-RNA tethering system, which specifically binds to said RNA-binding domain. As used herein the term “the RNA element of a protein-RNA tethering system which specifically binds to said RNA-binding domain” relates to an RNA sequence, usually forming a stem-loop, which is able to bind with high affinity to the corresponding RNA-binding domain of a protein-RNA tethering system.

Particularly, said DNA sequence is operatively linked to the promoter for a bacteriophage DNA-dependent RNA polymerase or to the promoter for said DNA-dependent RNA polymerase of the chimeric of the invention. In particular, when the RNA-binding domain of a protein-RNA tethering system is the RNA-binding domain of the lambdoid N antitermination protein-RNA tethering systems, the element, which specifically binds to said RNA-binding domain can be a boxBL and/or a boxBR stem loop RNA structure (Das 1993, Greenblatt, Nodwell et al. 1993, Friedman and Court 1995).

In particular, said DNA sequence is operatively linked to the promoter for a bacteriophage DNA-dependent RNA polymerase or to the promoter for said DNA-dependent RNA polymerase of the chimeric of the invention and covalently linked at its 3′ terminal end to at least one, preferably at least two, at least three and more preferably at least four sequences encoding the element which specifically binds to said RNA-binding domain.

In particular, said method according to the invention further comprises the step of contacting said DNA sequence encoding the RNA molecule with the enzyme of the invention. For example, said DNA sequence can be operatively linked to the promoter for a bacteriophage DNA-dependent RNA polymerase or to the promoter for said DNA-dependent RNA polymerase of the chimeric of the invention and covalently linked at its 3′ terminal end to at least one sequence encoding the element which specifically binds to said RNA-binding domain covalently linked to a poly(A) track sequence consisting of at least 10, in particular at least 20, 30, and more particularly at least 40 deoxyadenosine residues. Also, PolyA signal sequences can be used that recruit polyadenylation enzymes.

In particular, said poly(A) track sequence can be covalently linked at its 3′ terminal end to a self-cleaving RNA sequence and optionally to a transcription stop sequence. Said self-cleaving RNA sequence can be the self-cleaving RNA sequence from the group comprising the genomic pseudoknot ribozyme of the hepatitis D virus (Genbank accession number AJ000558.1), antigenomic hepatitis-D Virus pseudoknot ribozyme (Genbank accession number AJ000558.1), tobacco Ringspot Virus satellite hairpin ribozyme (Genbank accession number NC_003889.1) or artificial short hairpin RNA (shRNA).

In particular, said method according to the invention can further comprise the step of introducing in the host cell said DNA sequence and/or the nucleic acid according to the invention, using well-known methods by one skilled in the art like by transfection using calcium phosphate, by electroporation or by mixing a cationic lipid with DNA to produce liposomes.

In one embodiment, said method according to the invention further comprises the step of inhibiting, in particular silencing, preferably by siRNA (small interfering RNA), miRNA (microRNA) or shRNA, the cellular transcription and post-transcriptional machineries of said host cell.

In one embodiment, said method according to the invention further comprises the step of inhibiting the expression of the endogenous DNA-dependent RNA polymerase and/or the endogenous capping enzyme in said host cell.

The step of inhibiting the expression of the endogenous DNA-dependent RNA polymerase and/or the endogenous capping enzyme in said host cell can be implemented by any techniques well known to one skilled in the art, including but not limiting to siRNA techniques that target said endogenous DNA-dependent RNA polymerase and/or the endogenous capping enzyme, antisense RNA techniques that target said endogenous DNA-dependent RNA polymerase and/or the endogenous capping enzyme, shRNA techniques that target said endogenous DNA-dependent RNA polymerase and/or the endogenous capping enzyme.

In addition to siRNA (or shRNA), other inhibitory sequences might be also considered for the same purpose including DNA or RNA antisense (Liu and Carmichael 1994, Dias and Stein 2002), hammerhead ribozyme (Salehi-Ashtiani and Szostak 2001), hairpin ribozyme (Lian, De Young et al. 1999) or chimeric snRNA U1-antisense targeting sequence (Fortes, Cuevas et al. 2003). In addition, other cellular target genes might be considered for inhibition, including other genes involved in the cellular transcription (e.g. other subunits of the RNA polymerase II or transcription factors), post-transcriptional processing (e.g. other subunit of the capping enzyme, as well as polyadenylation or spliceosome factors), and mRNA nuclear export pathway.

In one embodiment of the method according to the invention, said RNA molecule can encode a polypeptide of therapeutic interest.

In another embodiment, said RNA molecule can be a non-coding RNA molecule selected in the group comprising siRNA, ribozyme, shRNA and antisense RNA. In particular, said DNA sequence can encode an RNA molecule selected in the group consisting of mRNA, non-coding RNA, particularly siRNA, ribozyme, shRNA and antisense RNA.

The invention also relates to the use of an engineered enzyme according to the invention as a capping enzyme and preferably a pol (A) polymerase and a DNA-dependent RNA polymerase.

The invention also relates to a kit for the production of a RNA molecule with 5′-terminal cap, in particular 5′-terminal m7GpppN cap, comprising at least one engineered enzyme according to the invention as defined above, and/or an isolated nucleic acid molecule and/or a group of nucleic acid molecule according to the invention as defined above, and/or a vector according to the invention as defined above, or a protein comprising the engineered enzymes disclosed herein.

Advantageously, the kit or the compositions of the invention can be used as an orthogonal gene expression system. As used herein, the term “orthogonal” designate biological systems whose basic structures are independent and generally originates from different species.

The invention also relates to an engineered enzyme according to the invention, an isolated nucleic acid molecule according to the invention, a group of nucleic acid molecule according to the invention or a vector according to the invention, for its use in the prevention and/or treatment of human or animal pathologies, preferably by means of gene therapy.

The invention also relates to a pharmaceutical composition comprising a chimeric enzyme according to the invention, and/or an isolated nucleic acid molecule according to the invention and/or a group of nucleic acid molecule according to the invention, and/or a vector according to the invention. Preferably, said pharmaceutical composition according to the invention is formulated in a pharmaceutical acceptable carrier.

Pharmaceutical acceptable carriers are well known by one skilled in the art.

The pharmaceutical composition according to the invention can further comprise at least one DNA sequence of interest, wherein said DNA sequence is operatively linked to a promoter for said catalytic domain of a DNA-dependent RNA polymerase and covalently linked to at least one sequence encoding the element which specifically binds to said RNA-binding domain.

Such components (in particular selected in the group consisting of a chimeric enzyme according to the invention, an isolated nucleic acid molecule according to the invention, a vector according to the invention and at least one DNA sequence of interest) can be present in the pharmaceutical composition or medicament according to the invention in a therapeutically amount (active and non-toxic amount).

Such therapeutically amount can be determined by one skilled in the art by routine tests including assessment of the effect of administration of said components on the pathologies and/or disorders which are sought to be prevent and/or to be treated by the administration of said pharmaceutical composition or medicament according to the invention.

For example, such tests can be implemented by analyzing both quantitative and qualitative effect of the administration of different amounts of said aforementioned components (in particular selected in the group consisting of a chimeric enzyme according to the invention, an isolated nucleic acid molecule according to the invention, a vector according to the invention and at least one DNA sequence of interest) on a set of markers (biological and/or clinical) characteristics of said pathologies and/or of said disorders, in particular from a biological sample of a subject.

The invention also relates to a therapeutic method comprising the administration of an engineered enzyme according to the invention, and/or an isolated nucleic acid molecule according to the invention, and/or a group of nucleic acid molecule according to the invention and/or a vector according to the invention in a therapeutically amount to a subject in need thereof. The therapeutic method according to the invention can further comprise the administration of at least one DNA sequence of interest, wherein said DNA sequence is operatively linked to a promoter for said catalytic domain of a DNA-dependent RNA polymerase and covalently linked to at least one sequence encoding the element which specifically binds to said RNA-binding domain, in a therapeutically amount to a subject in need thereof.

Said engineered enzyme, nucleic acid molecule and/or said vector according to the invention can be administrated simultaneously, separately or sequentially of said DNA sequence of interest, in particular before said DNA sequence of interest.

The invention also relates to a pharmaceutical composition according to the invention for its use for the prevention and/or treatment of human or animal pathologies, in particular by means of gene therapy.

Said pathologies can be selected from the group consisting of pathologies, which can be improved by the administration of said at least one DNA sequence of interest.

The invention also relates to the use of an engineered enzyme according to the invention, and/or an isolated nucleic acid molecule according to the invention, and/or a group of nucleic acid molecule according to the invention and/or a vector according to the invention, for the preparation of a medicament for the prevention and/or treatment of human or animal pathologies, in particular by means of gene therapy.

The invention can further comprise an engineered enzyme according to the invention and/or at least one nucleic acid molecule according to the invention and/or a group of nucleic acid molecule according to the invention and/or a at least one vector comprising and/or expressing a nucleic acid molecule according to the invention; and at least one DNA sequence of interest, wherein said DNA sequence is operatively linked to a promoter for said catalytic domain of a DNA-dependent RNA polymerase and covalently linked to at least one sequence encoding the element which specifically binds to said RNA-binding domain, wherein said active ingredients are formulated for separate, simultaneous or sequential administration.

Said DNA sequence of interest can be an anti-oncogene (a tumor suppressor gene). Said DNA sequence of interest can encode a polypeptide of therapeutic interest or a non-coding RNA selected in the group comprising siRNA, ribozyme, shRNA and antisense RNA. Said polypeptide of therapeutic interest can be selected from, a monoclonal antibody or its fragments, a growth factor, a cytokine, a cell or nuclear receptor, a ligand, a coagulation factor, the CFTR protein, insulin, dystrophin, a hormone, an enzyme, an enzyme inhibitor, a polypeptide which has an antineoplastic effect, a polypeptide which is capable of inhibiting a bacterial, parasitic or viral, in particular HIV, infection, an antibody, a toxin, an immunotoxin. Preferably, the combination product according to the invention can be formulated in a pharmaceutical acceptable carrier. In one embodiment of the combination product according to the invention, said vector is administrated before said DNA sequence of interest.

The invention also relates to a combination product according to the invention for its use as a medicament in the prevention and/or treatment of human or animal pathologies, particularly by means of gene therapy. Said pathologies can be selected from the group consisting of pathologies, which can be improved by the administration of at least one DNA sequence of interest, as described above.

For example, said pathologies, as well as their clinical, biological or genetic subtypes, can be selected from the group comprising liver disorders (e.g. acute liver failure due to acetaminophen intoxication or other causes, prevention of liver failure post-hepatectomy, liver primary cancers including hepatoma or cholangiocarcinoma, nonalcoholic steatohepatitis, as well as liver monogenic disorders such as hemochromatosis, ornithine transarbamylase deficiency, argininosuccinatelyase deficiency, argininosuccinate synthetase 1, hemochromatosis or Wilson's disease), disorders due or associated to deficiencies of secreted proteins (e.g. lysosomal storage diseases such as Gaucher's disease, Niemann-Pick disease, Tay-Sacks or Sandhoff disease, Hunter syndrome, or Hurler disease; deficiencies of coagulation factors including factors VIIIc, IX, Von Willebrand, fibrinogen or other coagulation proteins, as well as colony stimulating factors including erythropoietin, granulocyte colony stimulating factor and thrombopoietin), cancers and their predisposition (e.g. breast, colorectal, pancreas, gastric, esophageal and lung cancers, as well as melanoma), malignant hemopathies (e.g. leukemias, Hodgkin's and non-Hodgkin's lymphomas, myeloma), hemoglobinopathies (e.g. sickle cell anemia, glucose-6-phosphate dehydrogenase deficiency) and thalassemias, autoimmune disorders (e.g. systemic lupus erythematosus, scleroderma, autoimmune hepatitis), cardiovascular disorders (e.g. cardiac rhythm and conduction disorders, hypertrophic cardiomyopathy, cardiovascular disease, or chronic cardiac failure), metabolic disorders (e.g. type I and type II diabetes mellitus and their complications, dyslipidemia, atherosclerosis and their complications), infectious disorders (e.g. AIDS, viral hepatitis B, viral hepatitis C, influenza flu, Zika, Ebola and other viral diseases; botulism, tetanus and other bacterial disorders; malaria and other parasitic disorders), muscular disorders (e.g. Duchenne muscular dystrophy and Steinert myotonic muscular dystrophy), respiratory diseases (e.g. cystic fibrosis, alpha-1 antitrypsin deficiency, acute respiratory distress syndrome, pulmonary arterial hypertension, pulmonary veno-occlusive disease), renal diseases (e.g. polycystic kidney disease, glomerulopathy), colorectal disorders (e.g. Crohn's disease and ulcerative colitis), ocular disorders especially retinal diseases (e.g. Leber's amaurosis, retinitis pigmentosa, age related macular degeneration), central nervous system disorders (e.g. Alzheimer's disease, Parkinson's disease, amyotrophic lateral sclerosis, multiple sclerosis, Huntington's disease, neurofibromatosis, adrenoleukodystrophy, bipolar disease, schizophrenia and autism), bone and joint disorders (e.g. rheumatoid arthritis, ankylosing spondylitis, osteoarthritis) and skin and connective tissue disorders (e.g. neurofibromatosis and psoriasis).

The invention also relates to a method for producing the chimeric enzyme according to the invention comprising the step of expressing in at least one host cell said nucleic acid molecule or said group of nucleic acid molecules encoding the chimeric enzyme of the invention in conditions allowing the expression of said nucleic acid molecule(s) in said host cell.

Also disclosed herein is a method of selecting one or more engineered enzymes comprising a non-eukaryotic polymerase component and a capping enzyme component, wherein the engineered enzyme comprises enhanced activity compared to a control, the method comprising: (a) creating nucleic acid encoding the one or more engineered enzyme variants, wherein said variants comprise a variant of a naturally occurring non-eukaryotic polymerase and a variant of a naturally occurring capping enzyme component; (b) integrating said nucleic acid encoding one or more engineered enzyme variants into a one or more eukaryotic cells, wherein said eukaryotic cells comprises a reporter, wherein said reporter is under the control of a polymerase promoter which is specific for the polymerase of the engineered enzyme, and further wherein the reporter is only expressed when it is capped by said capping enzyme; (c) expressing said nucleic acid encoding one or more engineered enzyme variants; and (d) determining which of the one or more variants confer enhanced activity compared to a control and selecting said engineered enzyme variant. An example of such a method is found in Example 1.

In one embodiment, the naturally occurring non-eukaryotic polymerase and naturally occurring capping enzyme component are not naturally occurring in the same organism. For example, the polymerase can be T7 RNA polymerase, and the capping enzyme can be NP868R. The polymerase and capping enzyme can be separated by a linker. Examples of these enzymes, as well as linkers thereof, are described herein. The variant encoding said fusion protein can also encode a nuclear localization signal (NLS), which can be at an N-terminus of said fusion protein. Again, such NLSs are described elsewhere herein. The eukaryotic cell can be a yeast cell, for example, such as Saccharomyces cerevisiae.

The nucleic acid encoding one or more engineered enzyme variants can under the control of a promoter. This allows for the practitioner to initiate expression of the fusion protein as desired. Such promoters are known to those of skill in the art, and an example includes a galactose-inducible promoter.

The reporter which is used to detect expression of the fusion protein can be found in a plasmid. Examples of such reporters are known to those of skill in the art. Having the reporter in a separate plasmid allows for customization of the system. The reporter plasmid can, for example, comprise a fluorescent molecule which can easily be detected upon expression of the desired product.

The desired fusion protein products can then be identified and isolated. Optionally, the can then be put through further rounds of selection. For example, desired fusion protein products can be further mutated to determine additional mutations which confer desired benefits. These further mutants can then be subjected to the method of selecting described above. This method can be carried out 2, 3, 4, 5, 6, 7, 8, 9, 10, 20, 30, 40, 50, 60, 70, 80, 90, or more times. As discussed in Example 1, the top 0.1, 0.2, 0.3, 0.4, 0.5, 0.6. 0.7, 0.8, 0.9, 1.0, 1.1, 1.2, 1.3, 1.4, 1.5, 1.6, 1.7, 1.8, 1.9, or 2.0%, or more, of desired fluorescent clones can be gated and selected for further rounds of directed evolution. ‘Sexual PCR’ can then be used to minimize deleterious mutations along the path of selecting fusion proteins. This can be done in a high throughput fashion, for example.

After a desired engineered enzyme (also termed “fusion protein” herein) has been identified and isolated, it can be sequenced. Methods of sequencing are known to those of skill in the art. This sequencing can occur in a high-throughput manner, or by fluorescent (Sanger method) sequencing.

Disclosed herein are engineered enzyme variants which are discovered as a result of the methods of selecting a fusion protein as described herein. Also disclosed are nucleic acid molecules which encode said fusion proteins.

The control used in the method described above can be the naturally occurring non-eukaryotic polymerase and/or the naturally occurring capping enzyme component which corresponds with the variant or variants. Other controls include, but are not limited to, non-functional proteins, proteins from other organisms, or mutated proteins from other rounds of selection.

Also disclosed herein is a system which makes use of the method for directed evolution described above. Therefore, described herein is a system for selecting one or more engineered enzymes comprising a non-eukaryotic polymerase component and a capping enzyme component, wherein the engineered enzyme comprises enhanced activity, the system comprising a transformed eukaryotic cell, wherein said eukaryotic cell comprises a reporter plasmid, wherein said reporter plasmid is under the control of a polymerase promoter which is specific for the polymerase of the engineered enzyme, and further wherein the reporter is only expressed when it is capped by said capping enzyme. The eukaryotic cell can designed for integration of one or more variant nucleic acids.

Further disclosed is a method of selecting one or more engineered enzymes comprising a non-eukaryotic polymerase component and a capping enzyme component, wherein the method comprises: a) providing nucleic acid encoding said engineered enzyme, wherein expression of the engineered enzyme is under control of a promoter, wherein said promoter is recognized by the non-eukaryotic polymerase of the engineered enzyme; b) placing the nucleic acid encoding the engineered enzyme under conditions suitable for its expression; and c) detecting mRNA produced by the engineered enzyme, and selecting said enzyme for further analysis. In this method, the polymerase of the engineered enzyme is controlling the promoter for its own expression. This allows for a “feedback loop” which can yield an mRNA product which can then be detected and/or quantified to determine efficiency of transcription, or total amount present, for example. Using this feedback loop, one can determine if the designed engineered enzyme is, indeed, functional. Further analysis can comprise sequencing or amplification of the mRNA which is produced. Methods of sequencing and amplification are described elsewhere herein. A separate reporter can be included as well. Said reporters are known to those of skill in the art.

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. Efforts have been made to ensure accuracy with respect to numbers (e.g., amounts, temperatures, etc.); however, some errors and deviations should be accounted for. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Example 1

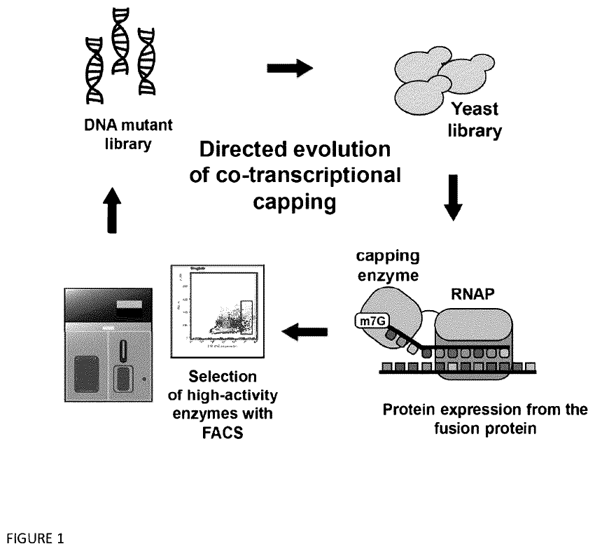

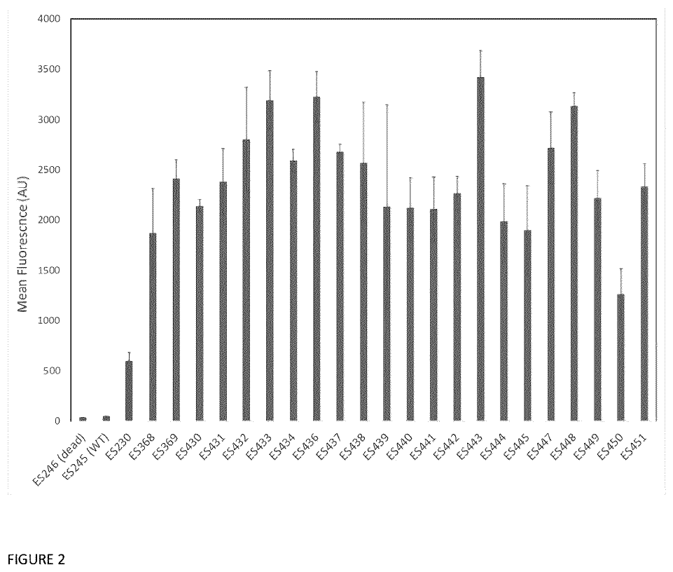

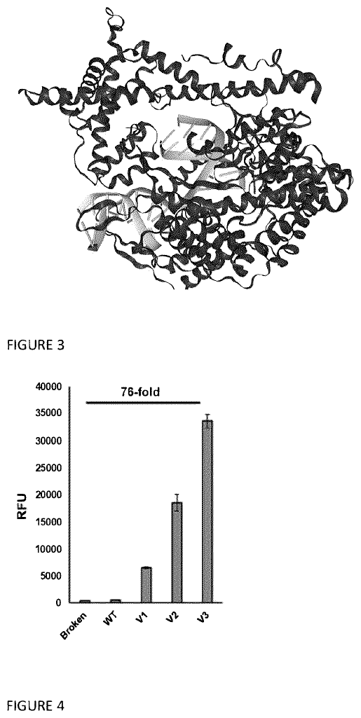

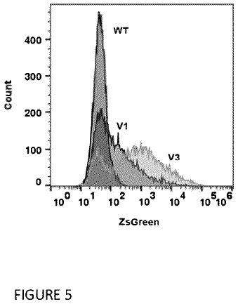

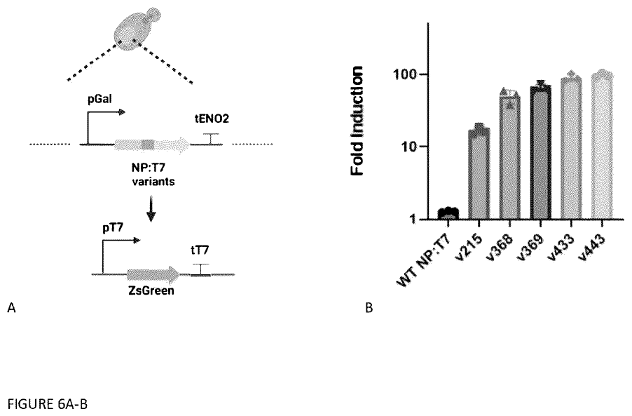

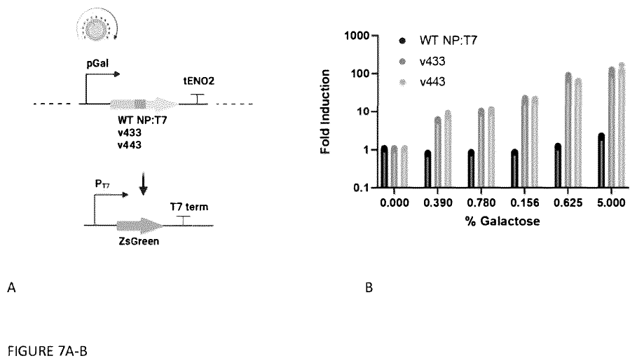

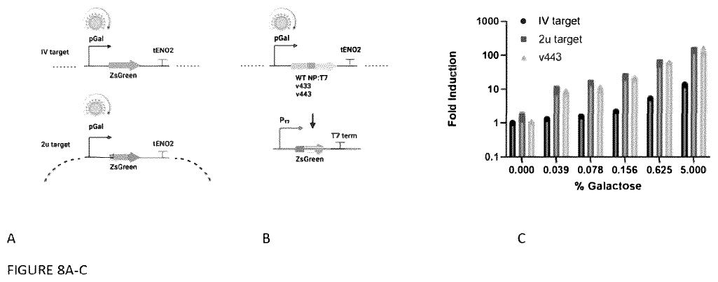

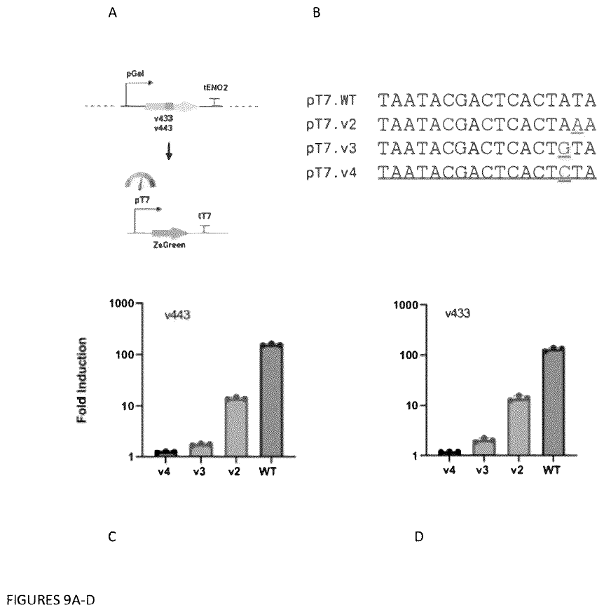

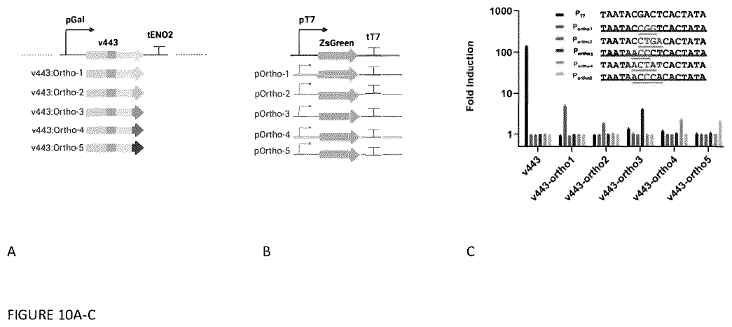

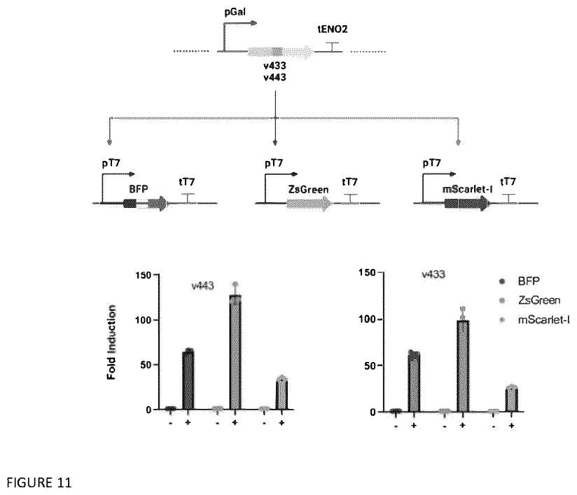

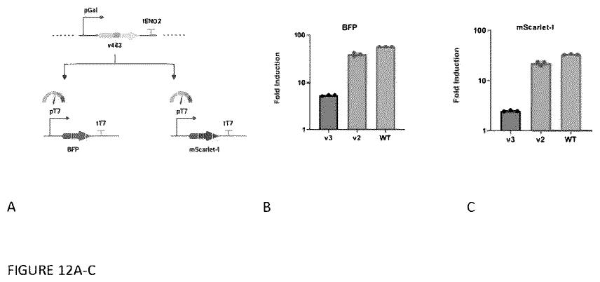

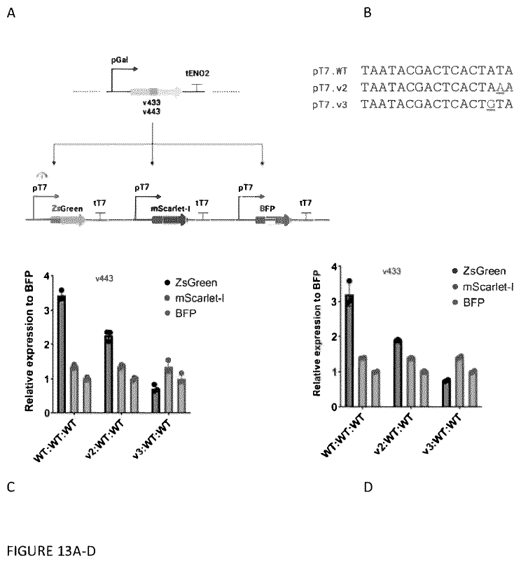

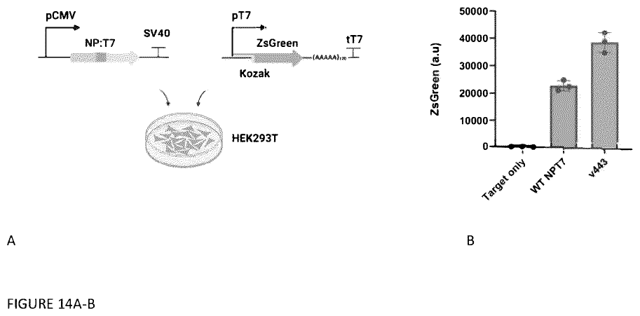

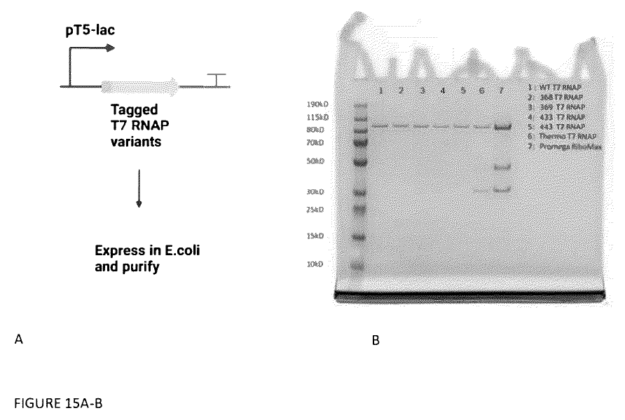

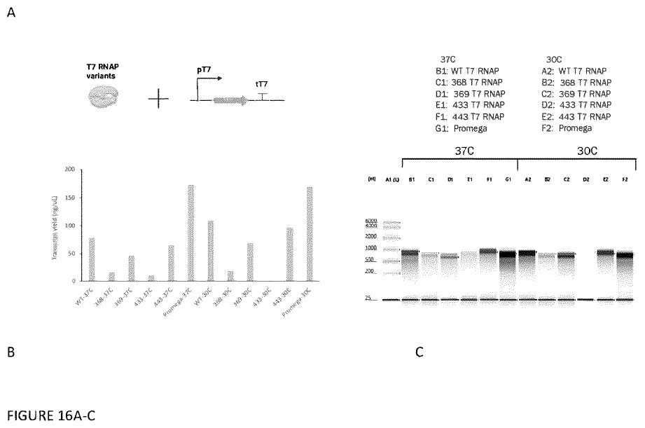

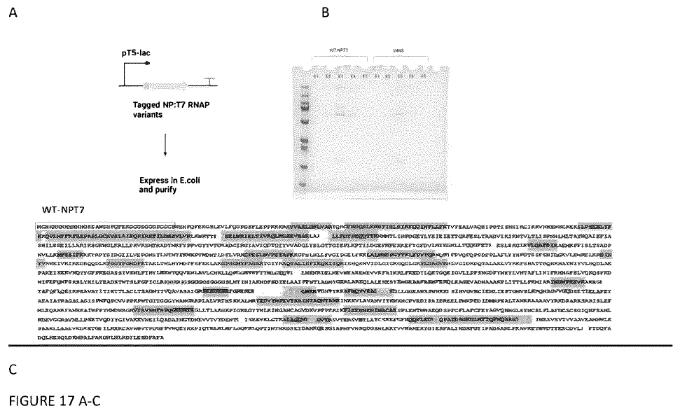

For further characterization of the fusion enzymes disclosed herein, and its ability to generate capped RNAs in the nucleus, a model eukaryote Saccharomyces cerevisiae was used. First to adapt this system for nuclear expression, a nuclear localization signal (SV40 NLS) was fused to the N-terminal of the fusion protein. The gene was placed under the control of the tightly regulated Gal promoter, and the entire cassette was integrated into the HO locus of S. cerevisiae. Next, a nuclear episomal reporter plasmid (2 micron) containing a fluorescent protein (ZsGreen1) under the control of an insulated T7 RNAP promoter followed by a polyadenylation signal (SV40) was built. Given the critical role of 5′ methylguanylate in the efficient translation of any mRNA in eukaryotic hosts, the levels of reporter protein expressed was used as a direct proxy for determining the transcriptional capping capacity of the enzyme (FIG. 1). As a negative control, a fusion protein containing the K282A mutation in NP868R was designed that abrogates the capping activity of the enzyme (ES246). When first characterized in yeast to drive expression of reporter (in the nucleus), the activity of the wild-type enzyme (ES245) was minimal compared to the negative control (FIG. 2).

It was hypothesized that this fusion enzyme can be engineered to generate capped transcripts more efficiently, which would likely result in higher protein expression. To engineer this enzyme, the entire gene (˜5.5 kbp) was mutagenized using error prone PCR. The library of variants was subcloned in E. coli and then integrated into the yeast strain containing the reporter plasmid. Following induction with galactose, the top 0.5-1% of the fluorescent clones were gated and selected for the next round of directed evolution. After numerous rounds of selection, specific variants containing numerous mutations in both proteins, showed greatly enhanced activity (about 75-fold higher protein expression compared to wild type enzyme) FIG. 2. ‘Sexual PCR’ was used to minimize deleterious mutations along the path of selecting fusion proteins. The complete list of mutations obtained from these variants are listed in Table 1. Additionally, machine learning tools such as convolution neural networks were used to identify more beneficial mutations in addition to those obtained from our selection (Table 2).

The capacity of the enzyme variants are characterized for improved protein production across other industrially relevant eukaryotic chassis organisms such as human cell lines and plants. In addition, the in vitro activity of the variants to generate 5′ capped RNAs are evaluated both as fusion and separate enzymes, and the performance is compared to wild T7 RNAP and the vaccinia capping enzyme for improved production of mRNA therapeutics and vaccines.

TABLE 1
Summary of all mutations from the
active variants (SEQ ID NOS: 6-24)

| | | |
|---|---|---|
| Position | WT | Mutation |

NLS

| | | |
|---|---|---|
| 9 | K | N |
| 10 | R | K |
| 10 | R | I |

NP868R

| | | |
|---|---|---|
| 15 | S | P |
| 59 | V | A |
| 160 | G | S |
| 184 | K | R |
| 279 | D | Y |
| 279 | D | N |
| 305 | Q | H |
| 351 | Y | C |
| 379 | N | D |
| 410 | T | A |
| 493 | L | M |
| 498 | Q | P |
| 534 | V | I |
| 544 | I | T |
| 553 | K | R |
| 578 | T | A |
| 606 | G | S |
| 620 | Q | R |
| 624 | Q | R |
| 654 | L | V |
| 690 | H | N |
| 740 | L | M |
| 752 | W | C |
| 753 | F | L |
| 757 | L | F |
| 798 | Q | K |
| 831 | K | E |
| 842 | S | P |
| 880 | N | S |

T7 RNAP

| | | |
|---|---|---|
| 905 | D | N |
| 921 | D | G |
| 963 | A | T |
| 982 | D | N |
| 983 | W | R |
| 1012 | I | T |
| 1024 | A | T |
| 1026 | N | K |
| 1058 | K | R |
| 1076 | A | V |
| 1078 | M | V |
| 1133 | G | C |
| 1156 | L | M |
| 1172 | P | T |
| 1178 | G | C |
| 1195 | H | R |
| 1199 | A | V |
| 1202 | R | H |
| 1227 | K | E |
| 1301 | N | K |
| 1390 | S | F |
| 1401 | D | G |
| 1407 | L | I |
| 1415 | G | E |
| 1478 | Q | P |
| 1543 | Q | R |
| 1551 | Q | R |
| 1581 | S | N |
| 1667 | H | R |
| 1754 | D | N |
| 1769 | D | G |

TABLE 2
51 positions to improve the stability
of the engineered fusion protein

| | | |
|---|---|---|
| 22 | ARG | 0.009591 |
| 30 | GLN | 0.018925 |
| 38 | GLU | 0.008359 |
| 45 | GLN | 0.023296 |
| 98 | GLN | 0.006222 |
| 100 | LEU | 0.004354 |
| 124 | ARG | 0.023449 |
| 143 | ARG | 0.019333 |
| 159 | MET | 0.016378 |
| 324 | ILE | 0.022483 |
| 396 | ASN | 0.009478 |
| 497 | THR | 0.01009 |
| 498 | GLN | 0.009947 |
| 502 | ASN | 0.006502 |
| 598 | MET | 0.008656 |
| 678 | ARG | 0.022212 |
| 679 | GLN | 0.024754 |
| 760 | GLN | 0.018512 |
| 832 | HIS | 0.021956 |
| 911 | LEU | 0.010602 |
| 914 | ILE | 0.018836 |
| 922 | HIS | 0.001159 |
| 952 | ARG | 0.006844 |
| 994 | ARG | 0.010308 |
| 1022 | THR | 0.012806 |
| 1025 | ASP | 0.010886 |
| 1027 | THR | 0.00784 |
| 1058 | LYS | 0.002599 |
| 1073 | TYR | 0.021673 |
| 1090 | LEU | 0.005642 |
| 1091 | LEU | 0.007834 |
| 1096 | TRP | 0.011899 |
| 1100 | HIS | 0.007672 |
| 1131 | VAL | 0.001925 |
| 1163 | PHE | 0.016407 |
| 1239 | TRP | 0.001118 |
| 1257 | MET | 0.00326 |
| 1264 | MET | 0.008112 |
| 1296 | MET | 0.021267 |
| 1476 | ILE | 0.007206 |
| 1487 | ASN | 0.006525 |
| 1500 | ILE | 0.025187 |
| 1539 | PHE | 0.006509 |
| 1561 | MET | 0.002735 |
| 1618 | CYS | 0.005185 |
| 1647 | LEU | 0.003287 |
| 1667 | HIS | 0.007599 |
| 1705 | ILE | 0.007482 |
| 1723 | VAL | 0.011494 |
| 1727 | MET | 0.004657 |
| 1734 | CYS | 0.011727 |

Lastly, it should be understood that while the present disclosure has been provided in detail with respect to certain illustrative and specific aspects thereof, it should not be considered limited to such, as numerous modifications are possible without departing from the broad spirit and scope of the present disclosure as defined in the appended claims.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

### REFERENCES

1. Jais P H, Decroly E, Jacquet E, Le Boulch M, Jais A, Jean-Jean O, et al. C3P3-G1: first generation of a eukaryotic artificial cytoplasmic expression system. Nucleic Acids Research. 2019; 47 (5): 2681-98.
2. Eaton H E, Kobayashi T, Dermody T S, Johnston R N, Jais P H, Shmulevitz M. African Swine Fever Virus NP868R Capping Enzyme Promotes Reovirus Rescue during Reverse Genetics by Promoting Reovirus Protein Expression, Virion Assembly, and RNA Incorporation into Infectious Virions. J Virol. 2017; 91 (11).

SEQUENCES
Wild Type NP868R: T7 RNAP
SEQ ID NO: 1
MFLEPPKKKRKVVASLDNLVARYQRCFNDQSLKNSTIELEIRFQQ
INFLLFKTVYEALVAQEIPSTISHSIRCIKKVHHENHCREKILPS
ENLYFKKQPLMFFKFSEPASLGCKVSLAIEQPIRKFILDSSVLVR
LKNRTTFRVSELWKIELTIVKQLMGSEVSAKLAAFKTLLFDTPEQ
QTTKNMMTLINPDGEYLYEIEIEYTGKPESLTAADVIKIKNTVLT
LISPNHLMLTAYHQAIEFIASHILSSEILLARIKSGKWGLKRLLP
RVKSMTKADYMKFYPPVGYYVTDKADGIRGIAVIQDTQIYVVADQ
LYSLGTTGIEPLKPTILDGEFMPEKKEFYGFDVIMYEGNLLTQQG
FETRIESLSKGIKVLQAFNIKAEMKPFISLTSADPNVLLKNFESI
FKKKTRPYSIDGIILVEPGNSYLNTNTFKWKPTWDNTLDFLVRKC
PESLNVPEYAPKKGFSLHLLFVGISGELFKKLALNWCPGYTKLFP
VTQRNQNYFPVQFQPSDFPLAFLYYHPDTSSFSNIDGKVLEMRCL
KREINYVRWEIVKIREDRQQDLKTGGYFGNDFKTAELTWLNYMDP
FSFEELAKGPSGMYFAGAKTGIYRAQTALISFIKQEIIQKISHQS
WVIDLGIGKGQDLGRYLDAGVRHLVGIDKDQTALAELVYRKFSHA
TTRQHKHATNIYVLHQDLAEPAKEISEKVHQIYGFPKEGASSIVS
NLFIHYLMKNTQQVENLAVLCHKLLQPGGMVWFTTMLGEQVLELL
HENRIELNEVWEARENEVVKFAIKRLFKEDILQETGQEIGVLLPF
SNGDFYNEYLVNTAFLIKIFKHHGFSLVQKQSFKDWIPEFQNFSK
SLYKILTEADKTWTSLFGFICLRKNGGGGSGGGGSGGGGSLNTIN
IAKNDFSDIELAAIPFNTLADHYGERLAREQLALEHESYEMGEAR
FRKMFERQLKAGEVADNAAAKPLITTLLPKMIARINDWFEEVKAK
RGKRPTAFQFLQEIKPEAVAYITIKTTLACLTSADNTTVQAVASA
IGRAIEDEARFGRIRDLEAKHFKKNVEEQLNKRVGHVYKKAFMQV
VEADMLSKGLLGGEAWSSWHKEDSIHVGVRCIEMLIESTGMVSLH
RQNAGVVGQDSETIELAPEYAEAIATRAGALAGISPMFQPCVVPP
KPWTGITGGGYWANGRRPLALVRTHSKKALMRYEDVYMPEVYKAI
NIAQNTAWKINKKVLAVANVITKWKHCPVEDIPAIEREELPMKPE
DIDMNPEALTAWKRAAAAVYRKDKARKSRRISLEFMLEQANKFAN
HKAIWFPYNMDWRGRVYAVSMFNPQGNDMTKGLLTLAKGKPIGKE
GYYWLKIHGANCAGVDKVPFPERIKFIEENHENIMACAKSPLENT
WWAEQDSPFCFLAFCFEYAGVQHHGLSYNCSLPLAFDGSCSGIQH
FSAMLRDEVGGRAVNLLPSETVQDIYGIVAKKVNEILQADAINGT
DNEVVTVTDENTGEISEKVKLGTKALAGQWLAYGVTRSVTKRSVM
TLAYGSKEFGFRQQVLEDTIQPAIDSGKGLMFTQPNQAAGYMAKL
IWESVSVTVVAAVEAMNWLKSAAKLLAAEVKDKKTGEILRKRCAV
HWVTPDGFPVWQEYKKPIQTRLNLMFLGQFRLQPTINTNKDSEID
AHKQESGIAPNFVHSQDGSHLRKTVVWAHEKYGIESFALIHDSFG
TIPADAANLFKAVRETMVDTYESCDVLADFYDQFADQLHESQLDK
MPALPAKGNLNLRDILESDFAFA
Nuclear Localization signal
SEQ ID NO: 2
MFLEPPKKKRKVV
T7 RNAP
SEQ ID NO: 3
NTINIAKNDFSDIELAAIPFNTLADHYGERLAREQLALEHESYEM
GEARFRKMFERQLKAGEVADNAAAKPLITTLLPKMIARINDWFEE
VKAKRGKRPTAFQFLQEIKPEAVAYITIKTTLACLTSADNTTVQA
VASAIGRAIEDEARFGRIRDLEAKHFKKNVEEQLNKRVGHVYKKA
FMQVVEADMLSKGLLGGEAWSSWHKEDSIHVGVRCIEMLIESTGM
VSLHRQNAGVVGQDSETIELAPEYAEAIATRAGALAGISPMFQPC
VVPPKPWTGITGGGYWANGRRPLALVRTHSKKALMRYEDVYMPEV
YKAINIAQNTAWKINKKVLAVANVITKWKHCPVEDIPAIEREELP
MKPEDIDMNPEALTAWKRAAAAVYRKDKARKSRRISLEFMLEQAN
KFANHKAIWFPYNMDWRGRVYAVSMFNPQGNDMTKGLLTLAKGKP
IGKEGYYWLKIHGANCAGVDKVPFPERIKFIEENHENIMACAKSP
LENTWWAEQDSPFCFLAFCFEYAGVQHHGLSYNCSLPLAFDGSCS
GIQHFSAMLRDEVGGRAVNLLPSETVQDIYGIVAKKVNEILQADA
INGTDNEVVTVTDENTGEISEKVKLGTKALAGQWLAYGVTRSVTK
RSVMTLAYGSKEFGFRQQVLEDTIQPAIDSGKGLMFTQPNQAAGY
MAKLIWESVSVTVVAAVEAMNWLKSAAKLLAAEVKDKKTGEILRK
RCAVHWVTPDGFPVWQEYKKPIQTRLNLMFLGQFRLQPTINTNKD
SEIDAHKQESGIAPNFVHSQDGSHLRKTVVWAHEKYGIESFALIH
DSFGTIPADAANLFKAVRETMVDTYESCDVLADFYDQFADQLHES
QLDKMPALPAKGNLNLRDILESDFAFA
Linker
SEQ ID NO: 4
GGGGSGGGGSGGGGSL
NP868R
SEQ ID NO: 5
ASLDNLVARYQRCFNDQSLKNSTIELEIRFQQINFLLFKTVYEAL
VAQEIPSTISHSIRCIKKVHHENHCREKILPSENLYFKKQPLMFF
KFSEPASLGCKVSLAIEQPIRKFILDSSVLVRLKNRTTFRVSELW
KIELTIVKQLMGSEVSAKLAAFKTLLFDTPEQQTTKNMMTLINPD
GEYLYEIEIEYTGKPESLTAADVIKIKNTVLTLISPNHLMLTAYH
QAIEFIASHILSSEILLARIKSGKWGLKRLLPRVKSMTKADYMKF
YPPVGYYVTDKADGIRGIAVIQDTQIYVVADQLYSLGTTGIEPLK
PTILDGEFMPEKKEFYGFDVIMYEGNLLTQQGFETRIESLSKGIK
VLQAFNIKAEMKPFISLTSADPNVLLKNFESIFKKKTRPYSIDGI
ILVEPGNSYLNTNTFKWKPTWDNTLDFLVRKCPESLNVPEYAPKK
GFSLHLLFVGISGELFKKLALNWCPGYTKLFPVTQRNQNYFPVQF
QPSDFPLAFLYYHPDTSSFSNIDGKVLEMRCLKREINYVRWEIVK
IREDRQQDLKTGGYFGNDFKTAELTWLNYMDPFSFEELAKGPSGM
YFAGAKTGIYRAQTALISFIKQEIIQKISHQSWVIDLGIGKGQDL
GRYLDAGVRHLVGIDKDQTALAELVYRKFSHATTRQHKHATNIYV
LHQDLAEPAKEISEKVHQIYGFPKEGASSIVSNLFIHYLMKNTQQ
VENLAVLCHKLLQPGGMVWFTTMLGEQVLELLHENRIELNEVWEA
RENEVVKFAIKRLFKEDILQETGQEIGVLLPFSNGDFYNEYLVNT
AFLIKIFKHHGFSLVQKQSFKDWIPEFQNFSKSLYKILTEADKTW
TSLFGFICLRKN
Variant ES 230
SEQ ID NO: 6
MFLEPPKKKIKVVASLDNLVARYQRCFNDQSLKNSTIELEIRFQQ
INFLLFKTVYEALVAQEIPSTISHSIRCIKKVHHENHCREKILPS
ENLYFKKQPLMFFKFSEPASLGCKVSLAIEQPIRKFILDSSVLVR
LKNRTTFRVSELWKIELTIVKQLMSSEVSAKLAAFKTLLFDTPEQ
QTTKNMMTLINPDGEYLYEIEIEYTGKPESLTAADVIKIKNTVLT
LISPNHLMLTAYHQAIEFIASHILSSEILLARIKSGKWGLKRLLP
RVKSMTKADYMKFYPPVGYYVTDKADGIRGIAVIQDTQIYVVADQ
LYSLGTTGIEPLKPTILDGEFMPEKKEFYGFDVIMYEGNLLTQQG
FETRIESLSKGIKVLQAFNIKAEMKPFISLTSADPNVLLKNFESI
FKKKTRPYSIDGIILVEPGNSYLNTNTFKWKPTWDNTLDFLVRKC
PESLNVPEYAPKKGFSLHLLFVGISGELFKKLALNWCPGYTKLFP
VTQRNQNYFPVQFQPSDFPLAFLYYHPDTSSFSNIDGKVLEMRCL
KREINYVRWEIVKIREDRQQDLKTGGYFGNDFKTAELTWLNYMDP
FSFEELAKGPSGMYFAGAKTGIYRAQTALISFIKQEIIQKISHQS
WVIDLGIGKGQDLGRYLDAGVRHLVGIDKDQTALAELVYRKFSHA
TTRQHKHATNIYVLHQDLAEPAKEISEKVHQIYGFPKEGASSIVS
NLFIHYLMKNTQQVENLAVLCHKLLQPGGMVWFTTMLGEQVLELL
HENRIELNEVWEARENEVVKFAIKRLFKEDILQETGQEIGVLLPF
SNGDFYNEYLVNTAFLIKIFEHHGFSLVQKQSFKDWIPEFQNFSK
SLYKILTEADKTWTSLFGFICLRKSGGGGSGGGGSGGGGSLNTIN
IAKNDFSDIELAAIPFNTLADHYGERLAREQLALEHESYEMGEAR
FRKMFERQLKAGEVADNAAAKPLITTLLPKMIARINNWFEEVKAK
RGKRPTAFQFLQEIKPEAVAYITIKTTLACLTSADNTTVQAVASA
IGRAIEDEARFGRIRDLEAKHFKKNVEEQLNKRVGHVYKKAFMQV
VEADMLSKGLLGGEAWSSWHKEDSIHVGVRCIEMLIESTGMVSLH
RQNAGVVGQDSETIELAPEYAEAIATRAGALAGISPMFQPCVVPP
KPWTGITCGGYWANGRRPLALVRTHSKKALMRYEDVYMPEVYKAI
NIAQNTAWKINKKVLAVANVITKWKHCPVEDIPAIEREELPMKPE
DIDMNPEALTAWKRAAAAVYRKDKARKSRRISLEFMLEQANKFAN
HKAIWFPYNMDWRGRVYAVSMFNPQGNDMTKGLLTLAKGKPIGKE
GYYWLKIHGANCAGVDKVPFPERIKFIEENHENIMACAKSPLENT
WWAEQDSPFCFLAFCFEYAGVQHHGLSYNCSLPLAFDGSCSGIQH
FSAMLRDEVGGRAVNLLPSETVQDIYGIVAKKVNEILQADAINGT
DNEVVTVTDENTGEISEKVKLGTKALAGQWLAYGVTRSVTKRSVM
TLAYGSKEFGFRRQVLEDTIQPAIDSGKGLMFTQPNQAAGYMAKL
IWESVSVTVVAAVEAMNWLKSAAKLLAAEVKDKKTGEILRKRCAV
HWVTPDGFPVWQEYKKPIQTRLNLMFLGQFRLQPTINTNKDSEID
ARKQESGIAPNFVHSQDGSHLRKTVVWAHEKYGIESFALIHDSFG
TIPADAANLFKAVRETMVDTYESCDVLADFYDQFADQLHESQLDK
MPALPAKGNLNLRDILESDFAFA
Variant ES 368
SEQ ID NO: 7
MFLEPPKKKKKVVASLDNLVARYQRCFNDQSLKNSTIELEIRFQQ
INFLLFKTVYEALVAQEIPSTISHSIRCIKKVHHENHCREKILPS
ENLYFKKQPLMFFKFSEPASLGCKVSLAIEQPIRKFILDSSVLVR
LKNRTTFRVSELWKIELTIVKQLMGSEVSAKLAAFKTLLFDTPEQ
QTTKNMMTLINPDGEYLYEIEIEYTGKPESLTAADVIKIKNTVLT
LISPNHLMLTAYHQAIEFIASHILSSEILLARIKSGKWGLKRLLP
RVKSMTKAYYMKFYPPVGYYVTDKADGIRGIAVIQDTQIYVVADQ
LYSLGTTGIEPLKPTILDGEFMPEKKEFYGFDVIMYEGNLLTQQG
FETRIESLSKGIKVLQAFNIKAEMKPFISLTSADPNVLLKNFESI
FKKKTRPYSIDGIILVEPGNSYLNTNTFKWKPTWDNTLDFLVRKC
PESLNVPEYAPKKGFSLHLLFVGISGELFKKLALNWCPGYTKLFP
VTQRNQNYFPVQFQPSDFPLAFLYYHPDTSSFSNIDGKVLEMRCL
KREINYVRWEIVKIREDRQQDLKTGGYFGNDFKTAELTWLNYMDP
FSFEELAKGPSGMYFAGAKTGIYRAQTALISFIKREIIQKISHQS
WVIDLGIGKGQDLGRYLDAGVRHLVGIDKDQTALAELVYRKFSHA
TTRQHKHATNIYVLHQDLAEPAKEISEKVHQIYGFPKEGASSIVS
NLFIHYLMKNTQQVENLAVMCHKLLQPGGMVWLTTMLGEQVLELL
HENRIELNEVWEARENEVVKFAIKRLFKEDILQETGQEIGVLLPF
SNGDFYNEYLVNTAFLIKIFKHHGFSLVQKQSFKDWIPEFQNFSK
SLYKILTEADKTWTSLFGFICLRKNGGGGSGGGGSGGGGSLNTIN
IAKNDFSDIELAAIPFNTLADHYGERLAREQLALEHESYEMGEAR
FRKMFERQLKAGEVADNAAAKPLITTLLPKMIARINDRFEEVKAK
RGKRPTAFQFLQEIKPEAVAYITIKTTLACLTSADNTTVQAVASA
IGRAIEDEARFGRIRDLEAKHFKKNVEEQLNKRVGHVYKKAFMQV
VEADMLSKGLLGGEAWSSWHKEDSIHVGVRCIEMLIESTGMVSLH
RQNAGVVGQDSETIELAPEYAEAIATRAGAMAGISPMFQPCVVPP
KPWTGITGGGYWANGRRPLALVRTHSKKALMRYEDVYMPEVYKAI
NIAQNTAWKINEKVLAVANVITKWKHCPVEDIPAIEREELPMKPE
DIDMNPEALTAWKRAAAAVYRKDKARKSRRISLEFMLEQANKFAN
HKAIWFPYNMDWRGRVYAVSMFNPQGNDMTKGLLTLAKGKPIGKE
GYYWLKIHGANCAGVDKVPFPERIKFIEENHENIMACAKFPLENT
WWAEQDSPFCFLAFCFEYAGVQHHGLSYNCSLPLAFDGSCSGIQH
FSAMLRDEVGGRAVNLLPSETVQDIYGIVAKKVNEILQADAINGT
DNEVVTVTDENTGEISEKVKLGTKALAGQWLAYGVTRSVTKRSVM
TLAYGSKEFGFRQQVLEDTIQPAIDSGKGLMFTQPNQAAGYMAKL
IWESVNVTVVAAVEAMNWLKSAAKLLAAEVKDKKTGEILRKRCAV
HWVTPDGFPVWQEYKKPIQTRLNLMFLGQFRLQPTINTNKDSEID
ARKQESGIAPNFVHSQDGSHLRKTVVWAHEKYGIESFALIHDSFG
TIPADAANLFKAVRETMVDTYESCDVLADFYDQFADQLHESQLDK
MPALPAKGNLNLRGILESDFAFA
Variant ES 369
SEQ ID NO: 8
MFLEPPKKNRKVVASLDNLVARYQRCFNDQSLKNSTIELEIRFQQ
INFLLFKTVYEALVAQEIPSTISHSIRCIKKVHHENHCREKILPS
ENLYFKKQPLMFFKFSEPASLGCKVSLAIEQPIRKFILDSSVLVR
LKNRTTFRVSELWKIELTIVKQLMGSEVSAKLAAFKTLLFDTPEQ
QTTKNMMTLINPDGEYLYEIEIEYTGKPESLTAADVIKIKNTVLT
LISPNHLMLTAYHQAIEFIASHILSSEILLARIKSGKWGLKRLLP
RVKSMTKANYMKFYPPVGYYVTDKADGIRGIAVIQDTQIYVVADQ
LYSLGTTGIEPLKPTILDGEFMPEKKEFYGFDVIMYEGNLLTQQG
FETRIESLSKGIKVLQAFDIKAEMKPFISLTSADPNVLLKNFESI
FKKKTRPYSIDGIILVEPGNSYLNTNTFKWKPTWDNTLDFLVRKC
PESLNVPEYAPKKGFSLHLLFVGISGELFKKLALNWCPGYTKLFP
VTQRNQNYFPVQFQPSDFPLAFLYYHPDTSSFSNIDGKVLEMRCL
KREINYVRWEIVRIREDRQQDLKTGGYFGNDFKTAELTWLNYMDP
FSFEELAKGPSGMYFAGAKTGIYRAQTALISFIKQEIIRKISHQS
WVIDLGIGKGQDLGRYLDAGVRHLVGIDKDQTALAELVYRKFSHA
TTRQHKHATNIYVLHQDLAEPAKEISEKVHQIYGFPKEGASSIVS
NLFIHYLMKNTQQVENLAVLCHKLLQPGGMVWFTTMLGEQVLELL
HENRIELNEVWEARENEVVKFAIKRLFKEDILQETGQEIGVLLPF
SNGDFYNEYLVNTAFLIKIFKHHGFSLVQKQSFKDWIPEFQNFSK
SLYKILTEADKTWTSLFGFICLRKNGGGGSGGGGSGGGGSLNTIN
IAKNDFSDIELAAIPFNTLAGHYGERLAREQLALEHESYEMGEAR
FRKMFERQLKAGEVADNAAAKPLITTLLPKMIARINDWFEEVKAK
RGKRPTAFQFLQEIKPEAVAYTTIKTTLACLTSTDNTTVQAVASA
IGRAIEDEARFGRIRDLEAKHFRKNVEEQLNKRVGHVYKKAFMQV
VEADMLSKGLLGGEAWSSWHKEDSIHVGVRCIEMLIESTGMVSLH
RQNAGVVCQDSETIELAPEYAEAIATRAGALAGISPMFQPCVVPP
KPWTGITGGGYWANGRRPLALVRTRSKKALMHYEDVYMPEVYKAI
NIAQNTAWKINKKVLAVANVITKWKHCPVEDIPAIEREELPMKPE
DIDMNPEALTAWKRAAAAVYRKDKARKSRRISLEFMLEQANKFAN
HKAIWFPYNMDWRGRVYAVSMFNPQGNDMTKGLLTLAKGKPIGKE
GYYWLKIHGANCAGVDKVPFPERIKFIEENHENIMACAKSPLENT
WWAEQGSPFCFLAFCFEYAGVQHHGLSYNCSLPLAFDGSCSGIQH
FSAMLRDEVGGRAVNLLPSETVQDIYGIVAKKVNEILQADAINGT
DNEVVTVTDENTGEISEKVKLGTKALAGQWLAYGVTRSVTKRSVM
TLAYGSKEFGFRQQVLEDTIRPAIDSGKGLMFTQPNQAAGYMAKL
IWESVSVTVVAAVEAMNWLKSAAKLLAAEVKDKKTGEILRKRCAV
HWVTPDGFPVWQEYKKPIQTRLNLMFLGQFRLQPTINTNKDSEID
ARKQESGIAPNFVHSQDGSHLRKTVVWAHEKYGIESFALIHDSFG
TIPADAANLFKAVRETMVDTYESCDVLADFYDQFADQLHESQLDK
MPALPAKGNLNLRGILESDFAFA
Variant ES-430
SEQ ID NO: 9
MFLEPPKKNRKVVASLDNLVARYQRCFNDQSLKNSTIELEIRFQQ
INFLLFKTVYEALVAQEIPSTISHSIRCIKKVHHENHCREKILPS
ENLYFKKQPLMFFKFSEPASLGCKVSLAIEQPIRKFILDSSVLVR
LKNRTTFRVSELWKIELTIVKQLMGSEVSAKLAAFKTLLFDTPEQ
QTTKNMMTLINPDGEYLYEIEIEYTGKPESLTAADVIKIKNTVLT
LISPNHLMLTAYHQATEFIASHILSSEILLARIKSGKWGLKRLLP
RVKSMTKANYMKFYPPVGYYVTDKADGIRGIAVIQDTQIYVVADQ
LYSLGTTGIEPLKPTILDGEFMPEKKEFYGFDVIMYEGNLLTQQG
FETRIESLSKGIKVLQAFDIKAEMKPFISLTSADPNVLLKNFESI
FKKKTRPYSIDGIILVEPGNSYLNTNTFKWKPTWDNTLDFLVRKC
PESLNVPEYAPKKGFSLHLLFVGISGELFKKLALNWCPGYTKLFP
VTQRNQNYFPVQFQPSDFPLAFLYYHPDTSSFSNIDGKVLEMRCL
KREINYVRWEIVRIREDRQQDLKTGGYFGNDFKTAELTWLNYMDP
FSFEELAKGPSGMYFAGAKTGIYRAQTALISFIKQEIIQKISHQS
WVIDLGIGKGQDLGRYLDAGVRHLVGIDKDQTALAELVYRKFSHA
TTRQHKHATNIYVLHQDLAEPAKEISEKVHQIYGFPKEGASSIVS
NLFIHYLMKNTQQVENLAVLCHKLLQPGGMVWFTTMLGEQVLELL
HENRIELNEVWEARENEVVKFAIKRLFKEDILQETGQEIGVLLPF
SNGDFYNEYLVNTAFLIKIFKHHGFSLVQKQSFKDWIPEFQNFSK
SLYKILTEADKTWTSLFGFICLRKNGGGGSGGGGSGGGGSLNTIN
IAKNDFSDIELAAIPFNTLADHYGERLAREQLALEHESYEMGEAR
FRKMFERQLKAGEVADNAAAKPLITTLLPKMIARINDWFEEVKAK
RGKRPTAFQFLQEIKPEAVAYITIKTTLACLTSADNTTVQAVASA
IGRAIEDEARFGRIRDLEAKHFRKNVEEQLNKRVGHVYKKAFMQV
VEADMLSKGLLGGEAWSSWHKEDSIHVGVRCIEMLIESTGMVSLH
RQNAGVVGQDSETIELAPEYAEAIETRAGALAGISPMFQPCVVPP
KPWTGITGGGYWANGRRPLALVRTRSKKALMHYEDVYMPEVYKAI
NIAQNTAWKINKKVLAVANVITKWKHCPVEDIPAIEREELPMKPE
DIDMNPEALTAWKRAAAAVYRKDKARKSRRISLEFMLEQANKFAN
HKAIWFPYNMDWRGRVYAVSMFNPQGNDMTKGLLTLAKGKPIGKE
GYYWLKIHGANCAGVDKVPFPERIKFIEENHENIMACAKSPLENT
WWAEQDSPFCFLAFCFEYAGVQHHGLSYNCSLPLAFDGSCSGIQH
FSAMLRDEVGGRAVNLLPSETVQDIYGIVAKKVNEILQADAINGT
DNEVVTVTDENTGEISEKVKLGTKALAGQWLAYGVTRSVTKRSVM
TLAYGSKEFGFRQQVLEDTIRPAIDSGKGLMFTQPNQAAGYMAKL
IWESVSVTVVAAVEAMNWLKSAAKLLAAEVKDKKTGEILRKRCAV
HWVTPDGFPVWQEYKKPIQTRLNLMFLGQFRLQPTINTNKDSEID
ARKQESGIAPNFVHSQDGSHLRKTVVWAHEKYGIESFALIHDSFG
TIPADAANLFKAVRETMVDTYESCDVLADFYDQFADQLHESQLDK
MPALPAKGNLNLRGILESDFAFA
Variant ES-431
SEQ ID NO: 10
MFLEPPKKNRKVVASLDNLVARYQRCFNDQSLKNSTIELEIRFQQ
INFLLFKTVYEALVAQEIPSTISHSIRCIKKVHHENHCREKILPS
ENLYFKKQPLMFFKFSEPASLGCKVSLAIEQPIRKFILDSSVLVR
LKNRTTFRVSELWKIELTIVKQLMGSEVSAKLAAFKTLLFDTPEQ
QTTKNMMTLINPDGEYLYEIEIEYTGKPESLTAADVIKIKNTVLT
LISPNHLMLTAYHQAIEFIASHILSSEILLARIKSGKWGLKRLLP
RVKSMTKANYMKFYPPVGYYVTDKADGIRGIAVIQDTQIYVVADQ
LYSLGTTGIEPLKPTILDGEFMPEKKEFYGFDVIMYEGNLLTQQG
FETRIESLSKGIKVLQAFNIKAEMKPFISLTSADPNVLLKNFESI
FKKKTRPYSIDGIILVEPGNSYLNTNTFKWKPTWDNTLDFLVRKC
PESLNVPEYAPKKGFSLHLLFVGISGELFKKLALNWCPGYTKLFP
VTQRNQNYFPVQFQPSDFPLAFLYYHPDTSSFSNIDGKVLEMRCL
KREINYVRWEIVKIREDRQQDLKTGGYFGNDFKTAELTWLNYMDP
FSFEELAKGPSGMYFAGAKTGIYRAQTALISFIKQEIIRKISHQS
WVIDLGIGKGQDLGRYLDAGVRHLVGIDKDQTALAELVYRKFSHA
TTRQHKHATNIYVLHQDLAEPAKEISEKVHQIYGFPKEGASSIVS
NLFIHYLMKNTQQVENLAVMCHKLLQPGGMVWFTTMLGEQVLELL
HENRIELNEVWEARENEVVKFAIKRLFKEDILQETGQEIGVLLPF
SNGDFYNEYLVNTAFLIKIFKHHGFSLVQKQSFKDWIPEFQNFSK
SLYKILTEADKTWTSLFGFICLRKNGGGGSGGGGSGGGGSLNTIN
IAKNDSSDIELAAIPFNTLADHYGERLAREQLALEHESYEMGEAR
FRKMFERQLKAGEVADNAAAKPLITTLLPKMIARINDRFEEVKAK
RGKRPTAFQFLQEIKPEAVAYITIKTTLACLTSADNTTVQAVASA
IGRAIEDEARFGRIRDLEAKHFKKNVEEQLNKRVGHVYKKAFMQV
VEADMLSKGLLGGEAWSSWHKEDSIHVGVRCIEMLIESTGMVSLH
RQNAGVVGQDSETIELAPEYAEAIATRAGAMAGISPMFQPCVVPP
KPWTGITGGGYWANGRRPLALVRTRSKKALMHYEDVYMPEVYKAI
NIAQNTAWKINKKVLAVANVITKWKHCPVEDIPAIEREELPMKPE
DIDMNPEALTAWKRAAAAVYRKDKARKSRRISLEFMLEQANKFAN
HKAIWFPYNMDWRGRVYAVSMFNPQGNDMTKGLLTLAKGKPIGKE
GYYWLKIHGANCAGVDKVPFPERIKFIEENHENIMACAKSPLENT
WWAEQDSPFCFLAFCFEYAGVQHHGLSYNCSLPLAFDGSCSGIQH
FSAMLRDEVGGRAVNLLPSETVQDIYGIVAKKVNEILQADAINGT
DNEVVTVTDENTGEISEKVKLGTKALAGQWLAYGVTRSVTKRSVM
TLAYGSKEFGFRQQVLEDTIQPAIDSGKGLMFTQPNQAAGYMAKL
IWESVNVTVVAAVEAMNWLKSAAKLLAAEVKDKKTGEILRKRCAV
HWVTPDGFPVWQEYKKPIQTRLNLMFLGQFRLQPTINTNKDSEID
ARKQESGIAPNFVHSQDGSHLRKTVVWAHEKYGIESFALIHDSFG
TIPADAANLFKAVRETMVDTYESCDVLADFYDQFADQLHESQLDK
MPALPAKGNLNLRDILESDFAFA
Variant ES-432
SEQ ID NO: 11
MFLEPPKKNRKVVASLDNLVARYQRCFNDQSLKNSTIELEIRFQQ
INFLLFKTVYEALVAQEIPSTISHSIRCIKKVHHENHCREKILPS
ENLYFKKQPLMFFKFSEPASLGCKVSLAIEQPIRKFILDSSVLVR
LKNRTTFRVSELWKIELTIVKQLMGSEVSAKLAAFKTLLFDTPEQ
QTTKNMMTLINPDGEYLYEIEIEYTGKPESLTAADVIKIKNTVLT
LISPNHLMLTAYHQAIEFIASHILSSEILLARIKSGKWGLKRLLP
RVKSMTKANYMKFYPPVGYYVTDKADGIRGIAVIQDTQIYVVADQ
LYSLGTTGIEPLKPTILDGEFMPEKKEFYGFDVIMYEGNLLTQQG
FETRIESLSKGIKVLQAFDIKAEMKPFISLTSADPNVLLKNFEFI
FKKKTRPYSIDGIILVEPGNSYLNTNTFKWKPTWDNTLDFLVRKC
PESLNVPEYAPKKGFSLHLLFVGISGELFKKLALNWCPGYTKLFP
VTQRNQNYFPVQFQPSDFPLAFLYYHPDTSSFSNIDGKVLEMRCL
KREINYVRWEIVRIREDRQQDLKTGGYFGNDFKTAELTWLNYMDP
FSFEELAKGPSGMYFAGAKTGIYRAQTALISFIKQEIIRKISHQS
WVIDLGIGKGQDLGRYLDAGVRHLVGIDKDQTALAELVYRKFSHA
TTRQHKHATNIYVLHQDLAEPAKEISEKVHQIYGFPKEGASSIVS
NLFIHYLMKNTQQVENLAVLCHKLLQPGGMVWFTTMLGEQVLELL
HENRIELNEVWEARENEVVKFAIKRLFKEDILQETGQEIGVLLPF
SNGDFYNEYLVNTAFLIKIFKHHGFSLVQKQSFKDWIPEFQNFSK
SLYKILTEADKTWTSLFGFICLRKNGGGGSGGGGSGGGGSLNTIN
IAKNDFSDIELAAIPFNTLADHYGERLAREQLALEHESYEMGEAR
FRKMFERQLKAGEVADNAAAKPLITTLLPKMIARINDWFEEVKAK
RGKRPTAFQFLQEIKPEAVAYTTIKTTLACLTSADNTTVQAVASA
IGRAIEDEARFGRIRDLEAKHFKKNVEEQLNKRVGHVYKKAFMQV
VEADMLSKGLLGGEAWSSWHKEDSIHVGVRCIEMLIESTGMVSLH
RQNAGVVCQDSETIELAPEYAEAIATRAGALAGISPMFQPCVVPP
KPWTGITGGGYWANGRRPLALVRTRSKKALMHYEDVYMPEVYKAI
NIAQNTAWKINKKVLAVANVITKWKHCPVEDIPAIEREELPMKPE
DIDMNPEALTAWKRAAAAVYRKDKARKSRRISLEFMLEQANKFAN
HKAIWFPYNMDWRGRVYAVSMFNPQGNDMTKGLLTLAKGKPIGKE
GYYWLKIHGANCAGVDKVPFPERIKFIEENHENIMACAKSPLENT
WWAEQDSPFCFLAFCFEYAGVQHHGLSYNCSLPLAFDGSCSGIQH
FSAMLRDEVGGRAVNLLPSETVQDIYGIVAKKVNEILQADAINGT
DNEVVTVTDENTGEISEKVKLGTKALAGQWLAYGVTRSVTKRSVM
TLAYGSKEFGFRQQVLEDTIRPAIDSGKGLMFTQPNQAAGYMAKL
IWESVSVTVVAAVEAMNWLKSAAKLLAAEVKDKKTGEILRKRCAV
HWVTPDGFPVWQEYKKPIQTRLNLMFLGQFRLQPTINTNKDSEID
ARKQESGIAPNFVHSQDGSHLRKTVVWAHEKYGIESFALIHDSFG
TIPADAANLFKAVRETMVDTYESCDVLADFYDQFADQLHESQLDK
MPALPAKGNLNLRGILESDFAFA
Variant ES-433
SEQ ID NO: 12
MFLEPPKKNRKVVASLDNLVARYQRCFNDQSLKNSTIELEIRFQQ
INFLLFKTVYEALVAQEIPSTISHSIRCIKKVHHENHCREKILPS
ENLYFKKQPLMFFKFSEPASLGCKVSLAIEQPIRKFILDSSVLVR
LKNRTTFRVSELWKIELTIVKQLMGSEVSAKLAAFKTLLFDTPEQ
QTTKNMMTLINPDGEYLYEIEIEYTGKPESLTAADVIKIKNTVLT
LISPNHLMLTAYHQAIEFIASHILSSEILLARIKSGKWGLKRLLP
RVKSMTKANYMKFYPPVGYYVTDKADGIRGIAVIQDTQIYVVADQ
LYSLGTTGIEPLKPTILDGEFMPEKKEFYGFDVIMYEGNLLTQQG
FETRIESLSKGIKVLQAFNIKAEMKPFISLTSADPNVLLKNFESI
FKKKTRPYSIDGIILVEPGNSYLNTNTFKWKPTWDNTLDFLVRKC
PESLNVPEYAPKKGFSLHLLFVGISGELFKKLALNWCPGYTKLFP
VTQRNQNYFPVQFQPSDFPLAFLYYHPDTSSFSNIDGKVLEMRCL
KREINYVRWETVKIREDRQQDLKTGGYFGNDFKTAELTWLNYMDP
FSFEELAKGPSGMYFAGAKTGIYRAQTALISFIKREIIRKISHQS
WVIDLGIGKGQDLGRYLDAGVRHLVGIDKDQTALAELVYRKFSHA
TTRQHKHATNIYVLHQDLAEPAKEISEKVHQIYGFPKEGASSIVS
NLFIHYLMKNTQQVENLAVMCHKLLQPGGMVWLTTMLGEQVLELL
HENRIELNEVWEARENEVVKFAIKRLFKEDILQETGQEIGVLLPF
SNGDFYNEYLVNTAFLIKIFKHHGFSLVQKQSFKDWIPEFQNFSK
SLYKILTEADKTWTSLFGFICLRKNGGGGSGGGGSGGGGSLNTIN
IAKNDFSDIELAAIPFNTLAGHYGERLAREQLALEHESYEMGEAR
FRKMFERQLKAGEVADNAAAKPLITTLLPKMIARINDRFEEVKAK
RGKRPTAFQFLQEIKPEAVAYITIKTTLACLTSADNTTVQAVASA
IGRAIEDEARFGRIRDLEAKHFRKNVEEQLNKRVGHVYKKAFMQV
VEADMLSKGLLGGEAWSSWHKEDSIHVGVRCIEMLIESTGMVSLH
RQNAGVVGQDSETIELAPEYAEAIATRAGAMAGISPMFQPCVVPP
KPWTGITGGGYWANGRRPLALVRTRSKKALMHYEDVYMPEVYKAI
NIAQNTAWKINKKVLAVANVITKWKHCPVEDIPAIEREELPMKPE
DIDMNPEALTAWKRAAAAVYRKDKARKSRRISLEFMLEQANKFAN
HKAIWFPYNMDWRGRVYAVSMFNPQGNDMTKGLLTLAKGKPIGKE
GYYWLKIHGANCAGVDKVPFPERIKFIEENHENIMACAKSPLENT
WWAEQDSPFCFLAFCFEYAGVQHHGLSYNCSLPLAFDGSCSGIQH
FSAMLRDEVGGRAVNLLPSETVQDIYGIVAKKVNEILQADAINGT
DNEVVTVTDENTGEISEKVKLGTKALAGQWLAYGVTRSVTKRSVM
TLAYGSKEFGFRQQVLEDTIRPAIDSGKGLMFTQPNQAAGYMAKL
IWESVNVTVVAAVEAMNWLKSAAKLLAAEVKDKKTGEILRKRCAV
HWVTPDGFPVWQEYKKPIQTRLNLMFLGQFRLQPTINTNKDSEID
ARKQESGIAPNFVHSQDGSHLRKTVVWAHEKYGIESFALIHDSFG
TIPADAANLFKAVRETMVDTYESCDVLADFYDQFADQLHESQLDK
MPALPAKGNLNLRDILESDFAFA
Variant ES-434
SEQ ID NO: 13
MFLEPPKKNRKVVASLDNLVARYQRCFNDQSLKNSTIELEIRFQQ
INFLLFKTVYEALVAQEIPSTISHSIRCIKKVHHENHCREKILPS
ENLYFKKQPLMFFKFSEPASLGCKVSLAIEQPIRKFILDSSVLVR
LKNRTTFRVSELWKIELTIVKQLMGSEVSAKLAAFKTLLFDTPEQ
QTTKNMMTLINPDGEYLYEIEIEYTGKPESLTAADVIKIKNTVLT
LISPNHLMLTAYHQAIEFIASHILSSEILLARIKSGKWGLKRLLP
RVKSMTKAYYMKFYPPVGYYVTDKADGIRGIAVIQDTQIYVVADQ
LYSLGTTGIEPLKPTILDGEFMPEKKEFYGFDVIMYEGNLLTQQG
FETRIESLSKGIKVLQAFNIKAEMKPFISLTSADPNVLLKNFESI
FKKKTRPYSIDGIILVEPGNSYLNTNTFKWKPTWDNTLDFLVRKC
PESLNVPEYAPKKGFSLHLLFVGISGELFKKLALNWCPGYTKLFP
VTQRNQNYFPVQFQPSDFPLAFLYYHPDTSSFSNIDGKVLEMRCL
KREINYVRWEIVKIREDRQQDLKTGGYFGNDFKTAELTWLNYMDP
FSFEELAKGPSGMYFAGAKTGIYRAQTALISFIKQEIIQKISHQS
WVIDLGIGKGQDLGRYLDAGVRHLVGIDKDQTALAELVYRKFSHA
TTRQHKHATNIYVLHQDLAEPAKEISEKVHQIYGFPKEGASSIVS
NLFIHYLMKNTQQVENLAVLCHKLLQPGGMVWFTTMLGEQVLELL
HENRIELNEVWEARENEVVKFAIKRLFKEDILQETGQEIGVLLPF
SNGDFYNEYLVNTAFLIKIFKHHGFSLVQKQSFKDWIPEFQNFSK
SLYKILTEADKTWTSLFGFICLRKNGGGGSGGGGSGGGGSLNTIN
IAKNDFSDIELAAIPFNTLADHYGERLAREQLALEHESYEMGEAR
FRKMFERQLKAGEVADNAAAKPLITTLLPKMIARINDWFEEVKAK
RGKRPTAFQFLQEIKPEAVAYTTIKTTLACLTSTDNTTVQAVASA
IGRAIEDEARFGRIRDLEAKHFKKNVEEQLNKRVGHVYKKAFMQV
VEADMLSKGLLGGEAWSSWHKEDSIHVGVRCIEMLIESTGMVSLH
RQNAGVVGQDSETIELAPEYAEAIATRAGALAGISPMFQPCVVPP
KPWTGITGGGYWANGRRPLALVRTRSKKALMRYEDVYMPEVYKAI
NIAQNTAWKINKKVLAVANVITKWKHCPVEDIPAIEREELPMKPE
DIDMNPEALTAWKRAAAAVYRKDKARKSRRISLEFMLEQANKFAN
HKAIWFPYNMDWRGRVYAVSMFNPQGNDMTKGLLTLAKGKPIGKE
GYYWLKIHGANCAGVDKVPFPERIKFIEENHENIMACAKFPLENT
WWAEQDSPFCFLAFCFEYAGVQHHGLSYNCSLPLAFDGSCSGIQH
FSAMLRDEVGGRAVNLLPSETVQDIYGIVAKKVNEILQADAINGT
DNEVVTVTDENTGEISEKVKLGTKALAGQWLAYGVTRSVTKRSVM
TLAYGSKEFGFRQQVLEDTIRPAIDSGKGLMFTQPNQAAGYMAKL
IWESVSVTVVAAVEAMNWLKSAAKLLAAEVKDKKTGEILRKRCAV
HWVTPDGFPVWQEYKKPIQTRLNLMFLGQFRLQPTINTNKDSEID
ARKQESGIAPNFVHSQDGSHLRKTVVWAHEKYGIESFALIHDSFG
TIPADAANLFKAVRETMVDTYESCDVLADFYDQFADQLHESQLDK
MPALPAKGNLNLRGILESDFAFA
Variant ES-436
SEQ ID NO: 14
MFLEPPKKNRKVVASLDNLVARYQRCFNDQSLKNSTIELEIRFQQ
INFLLFKTVYEALVAQEIPSTISHSIRCIKKVHHENHCREKILPS
ENLYFKKQPLMFFKFSEPASLGCKVSLAIEQPIRKFILDSSVLVR
LKNRTTFRVSELWKIELTIVKQLMGSEVSAKLAAFKTLLFDTPEQ
QTTKNMMTLINPDGEYLYEIEIEYTGKPESLTAADVIKIKNTVLT
LISPNHLMLTAYHQAIEFIASHILSSEILLARIKSGKWGLKRLLP
RVKSMTKAYYMKFYPPVGYYVTDKADGIRGIAVIQDTQIYVVADQ
LYSLGTTGIEPLKPTILDGEFMPEKKEFYGFDVIMYEGNLLTQQG
FETRIESLSKGIKVLQAFNIKAEMKPFISLTSADPNVLLKNFESI
FKKKARPYSIDGIILVEPGNSYLNTNTFKWKPTWDNTLDFLVRKC
PESLNVPEYAPKKGFSLHLLFVGISGELFKKLALNWCPGYTKLFP
VTQRNQNYFPVQFQPSDFPLAFLYYHPDTSSFSNIDGKVLEMRCL
KREINYVRWEIVKIREDRQQDLKTGGYFGNDFKTAELTWLNYMDP
FSFEELAKGPSGMYFAGAKTGIYRAQTALISFIKQEIIQKISHQS
WVIDLGIGKGQDLGRYLDAGVRHLVGIDKDQTALAELVYRKFSHA
TTRQHKHATNIYVLHQDLAEPAKEISEKVHQIYGFPKEGASSIVS
NLFIHYLMKNTQQVENLAVMCHKLLQPGGMVWLTTMLGEQVLELL
HENRIELNEVWEARENEVVKFAIKRLFKEDILQETGQEIGVLLPF
SNGDFYNEYLVNTAFLIKIFKHHGFSLVQKQSFKDWIPEFQNFSK
SLYKILTEADKTWTSLFGFICLRKNGGGGSGGGGSGGGGSLNTIN
IAKNDFSDIELAAIPFNTLADHYGERLAREQLALEHESYEMGEAR
FRKMFERQLKAGEVADNAAAKPLITTLLPKMIARINDRFEEVKAK
RGKRPTAFQFLQEIKPEAVAYITIKTTLACLTSTDNTTVQAVASA
IGRAIEDEARFGRIRDLEAKHFRKNVEEQLNKRVGHVYKKAFVQV
VEADMLSKGLLGGEAWSSWHKEDSIHVGVRCIEMLIESTGMVSLH
RQNAGVVCQDSETIELAPEYAEAIATRAGALAGISPMFQPCVVPP
KPWTGITGGGYWANGRRPLALVRTRSKKALMHYEDVYMPEVYKAI
NIAQNTAWKINKKVLAVANVITKWKHCPVEDIPAIEREELPMKPE
DIDMNPEALTAWKRAAAAVYRKDKARKSRRISLEFMLEQANKFAN
HKAIWFPYNMDWRGRVYAVSMFNPQGNDMTKGLLTLAKGKPIGKE
GYYWLKIHGANCAGVDKVPFPERIKFIEENHENIMACAKSPLENT
WWAEQDSPFCFLAFCFEYAGVQHHGLSYNCSLPLAFDGSCSGIQH
FSAMLRDEVGGRAVNLLPSETVQDIYGIVAKKVNEILQADAINGT
DNEVVTVTDENTGEISEKVKLGTKALAGQWLAYGVTRSVTKRSVM
TLAYGSKEFGFRQQVLEDTIQPAIDSGKGLMFTQPNQAAGYMAKL
IWESVSVTVVAAVEAMNWLKSAAKLLAAEVKDKKTGEILRKRCAV
HWVTPDGFPVWQEYKKPIQTRLNLMFLGQFRLQPTINTNKDSEID
ARKQESGIAPNFVHSQDGSHLRKTVVWAHEKYGIESFALIHDSFG
TIPADAANLFKAVRETMVDTYESCDVLADFYDQFADQLHESQLDK
MPALPAKGNLNLRGILESDFAFA
Variant ES-440
SEQ ID NO: 15
MFLEPPKKKKKVVASLDNLVARYQRCFNDQSLKNSTIELEIRFQQ
INFLLFKTVYEALVAQEIPSTISHSIRCIKKVHHENHCREKILPS
ENLYFKKQPLMFFKFSEPASLGCKVSLAIEQPIRKFILDSSVLVR
LKNRTTFRVSELWKIELTIVKQLMGSEVSAKLAAFKTLLFDTPEQ
QTTKNMMTLINPDGEYLYEIEIEYTGKPESLTAADVIKIKNTVLT
LISPNHLMLTAYHQAIEFIASHILSSEILLARIKSGKWGLKRLLP
RVKSMTKANYMKFYPPVGYYVTDKADGIRGIAVIQDTQIYVVADQ
LYSLGTTGIEPLKPTILDGEFMPEKKEFYGFDVIMYEGNLLTQQG
FETRIESLSKGIKVLQAFDIKAEMKPFISLTSADPNVLLKNFESI
FKKKTRPYSIDGIILVEPGNSYLNTNTFKWKPTWDNTLDFLVRKC
PESLNVPEYAPKKGFSLHLLFVGISGELFKKLALNWCPGYTKLFP
VTQRNQNYFPVQFQPSDFPLAFLYYHPDTSSFSNIDGKVLEMRCL
KREINYVRWEIVKIREDRQQDLKTGGYFGNDFKTAELTWLNYMDP
FSFEELAKGPSGMYFAGAKTGIYRAQTALISFIKQEIIRKISHQS
WVIDLGIGKGQDLGRYLDAGVRHLVGIDKDQTALAELVYRKFSHA
TTRQHKHATNIYVLHQDLAEPAKEISEKVHQIYGFPKEGASSIVS
NLFIHYLMKNTQQVENLAVMCHKLLQPGGMVWLTTMLGEQVLELL
HENRIELNEVWEARENEVVKFAIKRLFKEDILQETGQEIGVLLPF
SNGDFYNEYLVNTAFLIKIFKHHGFSLVQKQSFKDWIPEFQNFSK
SLYKILTEADKTWTSLFGFICLRKNGGGGSGGGGSGGGGSLNTIN
IAKNDFSDIELAAIPFNTLAGHYGERLAREQLALEHESYEMGEAR
FRKMFERQLKAGEVADNAAAKPLITTLLPKMIARINDWFEEVKAK
RGKRPTAFQFLQEIKPEAVAYTTIKTTLACLTSTDNTTVQAVASA
IGRAIEDEARFGRIRDLEAKHFKKNVEEQLNKRVGHVYKKAFMQV
VEADMLSKGLLGGEAWSSWHKEDSIHVGVRCIEMLIESTGMVSLR
RQNAGVVGQDSETIELAPEYAEAIATRAGALAGISPMFQPCVVPP
KPWTGITGGGYWANGRRPLALVRTHSKKALMRYEDVYMPEVYKAI
NIAQNTAWKINKKVLAVANVITKWKHCPVEDIPAIEREELPMKPE
DIDMNPEALTAWKRAAAAVYRKDKARKSRRISLEFMLEQANKFAN
HKAIWFPYNMDWRGRVYAVSMFNPQGNDMTKGLLTLAKGKPIGKE
GYYWLKIHGANCAGVDKVPFPERIKFIEENHENIMACAKFPLENT
WWAEQDSPFCFLAFCFEYAGVQHHGLSYNCSLPLAFDGSCSGIQH
FSAMLRDEVGGRAVNLLPSETVQDIYGIVAKKVNEILQADAINGT
DNEVVTVTDENTGEISEKVKLGTKALAGQWLAYGVTRSVTKRSVM
TLAYGSKEFGFRQQVLEDTIQPAIDSGKGLMFTQPNQAAGYMAKL
IWESVNVTVVAAVEAMNWLKSAAKLLAAEVKDKKTGEILRKRCAV
HWVTPDGFPVWQEYKKPIQTRLNLMFLGQFRLQPTINTNKDSEID
ARKQESGIAPNFVHSQDGSHLRKTVVWAHEKYGIESFALIHDSFG
TIPADAANLFKAVRETMVDTYESCDVLADFYDQFADQLHESQLDK
MPALPAKGNLNLRGILESDFAFA
Variant ES-441
SEQ ID NO: 16
MFLEPPKKNRKVVASLDNLVARYQRCFNDQSLKNSTIELEIRFQQ
INFLLFKTVYEALVAQEIPSTISHSIRCIKKVHHENHCREKILPS
ENLYFKKQPLMFFKFSEPASLGCKVSLAIEQPIRKFILDSSVLVR
LKNRTTFRVSELWKIELTIVKQLMGSEVSAKLAAFKTLLFDTPEQ
QTTKNMMTLINPDGEYLYEIEIEYTGKPESLTAADVIKIKNTVLT
LISPNHLMLTAYHQAIEFIASHILSSEILLARIKSGKWGLKRLLP
RVKSMTKAYYMKFYPPVGYYVTDKADGIRGIAVIQDTQIYVVADQ
LYSLGTTGIEPLKPTILDGEFMPEKKEFYGFDVIMYEGNLLTQQG
FETRIESLSKGIKVLQAFNIKAEMKPFISLTSADPNVLLKNFESI
FKKKTRPYSIDGIILVEPGNSYLNTNTFKWKPTWDNTLDFLVRKC
PESLNVPEYAPKKGFSLHLLFVGISGELFKKLALNWCPGYTKLFP
VTQRNQNYFPVQFQPSDFPLAFLYYHPDTSSFSNIDGKVLEMRCL
KREINYVRWEIVKIREDRQQDLKTGGYFGNDFKTAELTWLNYMDP
FSFEELAKGPSGMYFAGAKTGIYRAQTALISFIKQEIIQKISHQS
WVIDLGIGKGQDLGRYLDAGVRHLVGIDKDQTALAELVYRKFSHA
TTRQHKHATNIYVLHQDLAEPAKEISEKVHQIYGFPKEGASSIVS
NLFIHYLMKNTQQVENLAVMCHKLLQPGGMVWLTTMLGEQVLELL
HENRIELNEVWEARENEVVKFAIKRLFKEDILQETGQEIGVLLPF
SNGDFYNEYLVNTAFLIKIFKHHGFSLVQKQSFKDWIPEFQNFSK
SLYKILTEADKTWTSLFGFICLRKNGGGGSGGGGSGGGGSLNTIN
IAKNDFSDIELAAIPFNTLADHYGERLAREQLALEHESYEMGEAR
FRKMFERQLKAGEVADNAAAKPLITTLLPKMIARINDRFEEVKAK
RGKRPTAFQFLQEIKPEAVAYITIKTTLACLTSADNTTVQAVASA
IGRAVEDEARFGRIRDLEAKHFRKNVEEQLNKRVGHVYKKAFMQV
VEADMLSKGLLGGEAWSSWHKEDSIHVGVRCIEMLIESTGMVSLH
RQNAGVVGQDSETIELAPEYAEAIATRAGAMAGISPMFQPCVVPP
KPWTGITGGGYWANGRRPLALVRTHSKKALMRYEDVYMPEVYKAI
NIAQNTAWKINKKVLAVANVITKWKHCPVEDIPAIEREELPMKPE
DIDMNPEALTAWKRAAAAVYRKDKARKSRRISLEFMLEQANKFAN
HKAIWFSYNMDWRGRVYAVSMFNPQGNDMTKGLLTLAKGKPIGKE
GYYWLKIHGANCAGVDKVPFPERIKFIEENHENIMACAKFPLENT
WWAEQDSPFCFLAFCFEYAGVQHHGLSYNCSLPLAFDGSCSGIQH
FSAMLRDEVGGRAVNLLPSETVQDIYGIVAKKVNEILQADAINGT
DNEVVTVTDENTGEISEKVKLGTKALAGQWLAYGVTRSVTKRSVM
TLAYGSKEFGFRQQVLEDTIQPAIDSGKGLMFTQPNQAAGYMAKL
IWESVNVTVVAAVEAMNWLKSAAKLLAAEVKDKKTGEILRKRCAV
HWVTPDGFPVWQEYKKPIQTRLNLMFLGQFRLQPTINTNKDSEID
ARKQESGIAPNFVHSQDGSHLRKTVVWAHEKYGIESFALIHDSFG
TIPADAANLFKAVRETMVDTYESCDVLADFYDQFADQLHESQLDK
MPALPAKGNLNLRGILESDFAFA
Variant ES-442
SEQ ID NO: 17
MFLEPPKKNRKVVASLDNLVARYQRCFNDQSLKNSTIELEIRFQQ
INFLLFKTVYEALVAQEIPSTISHSIRCIKKVHHENHCREKILPS
ENLYFKKQPLMFFKFSEPASLGCKVSLAIEQPIRKFILDSSVLVR
LKNRTTFRVSELWKIELTIVKQLMGSEVSAKLAAFKTLLFDTPEQ
QTTKNMMTLINPDGEYLYEIEIEYTGKPESLTAADVIKIKNTVLT
LISPNHLMLTAYHQAIEFIASHILSSEILLARIKSGKWGLKRLLP
RVKSMTKANYMKFYPPVGYYVTDKADGIRGIAVIQDTQIYVVADQ
LYSLGTTGIEPLKPTILDGEFMPEKKEFYGFDVIMYEGNLLTQQG
FETRIESLSKGIKVLQAFNIKAEMKPFISLTSADPNVLLKNFESI
FKKKTRPYSIDGIILVEPGNSYLNTNTFKWKPTWDNTLDFLVRKC
PESLNVPEYAPKKGFSLHLLFVGISGELFKKLALNWCPGYTKLFP
VTQRNQNYFPVQFQPSDFPLAFLYYHPDTSSFSNIDGKVLEMRCL
KREINYVRWEIVKIREDRQQDLKTGGYFGNDFKTAELTWLNYMDP
FSFEELAKGPSGMYFAGAKTGIYRAQTALISFIKQEIIQKISHQS
WVIDLGIGKGQDLGRYLDAGVRHLVGIDKDQTALAELVYRKFSHA
TTRQHKHATNIYVLNQDLAEPAKEISEKVHQIYGFPKEGASSIVS
NLFIHYLMKNTQQVENLAVLCHKLLQPGGMVWFTTMLGEQVLELL
HENRIELNEVWEARENEVVKFAIKRLFKEDILQETGQEIGVLLPF
SNGDFYNEYLVNTAFLIKIFEHHGFSLVQKQSFKDWIPEFQNFSK
SLYKILTEADKTWTSLFGFICLRKNGGGGSGGGGSGGGGSLNTIN
IAKNDFSDIELAAIPFNTLAGHYGERLAREQLALEHESYEMGEAR
FRKMFERQLKAGEVADNAAAKPLITTLLPKMIARINDWFEEVKAK
RGKRPTAFQFLQEIKPEAVAYITIKTTLACLTSADNTTVQAVASA
IGRAIEDEARFGRIRDLEAKHFRKNVEEQLNKRVGHVYKKAFMQV
VEADMLSKGLLGGEAWSSWHKEDSIHVGVRCIEMLIESTGMVSLH
RQNAGVVCQDSETIELAPEYAEAIATRAGALAGISPMFQPCVVPP
KPWTGITGGGYWANGRRPLALVRTRSKKALMHYEDVYMPEVYKAI
NIAQNTAWKINKKVLAVANVITKWKHCPVEDIPAIEREELPMKPE
DIDMNPEALTAWKRAAAAVYRKDKARKSRRISLEFMLEQANKFAN
HKAIWFPYNMDWRGRVYAVSMFNPQGNDMTKGLLTLAKGKPIGKE
GYYWLKIHGANCAGVDKVPFPERIKFIEENHENIMACAKSPLENT
WWAEQGSPFCFLAFCFEYAGVQHHGLSYNCSLPLAFDGSCSGIQH
FSAMLRDEVGGRAVNLLPSETVQDIYGIVAKKVNEILQADAINGT
DNEVVTVTDENTGEISEKVKLGTKALAGQWLAYGVTRSVTKRSVM
TLAYGSKEFGFRQQVLEDTIRPAIDSGKGLMFTQPNQAAGYMAKL
IWESVSVTVVAAVEAMNWLKSAAKLLAAEVKDKKTGEILRKRCAV
HWVTPDGFPVWQEYKKPIQTRLNLMFLGQFRLQPTINTNKDSEID
ARKQESGIAPNFVHSQDGSHLRKTVVWAHEKYGIESFALIHDSFG
TIPADAANLFKAVRETMVDTYESCDVLADFYDQFADQLHESQLDK
MPALPAKGNLNLRDILESDFAFA
Variant ES-443
SEQ ID NO: 18
MFLEPPKKNRKVVASLDNLVARYQRCFNDQSLKNSTIELEIRFQQ
INFLLFKTVYEALVAQEIPSTISHSIRCIKKVHHENHCREKILPS
ENLYFKKQPLMFFKFSEPASLGCKVSLAIEQPIRKFILDSSVLVR
LKNRTTFRVSELWKIELTIVKQLMGSEVSAKLAAFKTLLFDTPEQ
QTTKNMMTLINPDGEYLYEIEIEYTGKPESLTAADVIKIKNTVLT
LISPNHLMLTAYHQAIEFIASHILSSEILLARIKSGKWGLKRLLP
RVKSMTKANYMKFYPPVGYYVTDKADGIRGIAVIQDTQIYVVADQ
LYSLGTTGIEPLKPTILDGEFMPEKKEFYGFDVIMYEGNLLTQQG
FETRIESLSKGIKVLQAFNIKAEMKPFISLTSADPNVLLKNFESI
FKKKTRPYSIDGIILVEPGNSYLNTNTFKWKPTWDNTLDFLVRKC
PESLNVPEYAPKKGFSLHLLFVGISGELFKKLALNWCPGYTKLFP
VTQRNQNYFPVQFQPSDFPLAFLYYHPDTSSFSNIDGKVLEMRCL
KREINYVRWEIVKIREDRQQDLKTGGYFGNDFKTAELTWLNYMDP
FSFEELAKGPSGMYFAGAKTGIYRAQTALISFIKQEIIQKISHQS
WVIDLGIGKGQDLGRYLDAGVRHLVGIDKDQTALAELVYRKFSHA
TTRQHKHATNIYVLHQDLAEPAKEISEKVHQIYGFPKEGASSIVS
NLFIHYLMKNTQQVENLAVLCHKLLQPGGMVWFTTMLGEQVLELL
HENRIELNEVWEARENEVVKFAIKRLFKEDILQETGQEIGVLLPF
SNGDFYNEYLVNTAFLIKIFKHHGFSLVQKQSFKDWIPEFQNFSK
SLYKILTEADKTWTSLFGFICLRKNGGGGSGGGGSGGGGSLNTIN
IAKNDFSDIELAAIPFNTLADHYGERLAREQLALEHESYEMGEAR
FRKMFERQLKAGEVADNAAAKPLITTLLPKMIARINDWFEEVKAK
RGKRPTAFQFLQEIKPEAVAYITIKTTLACLTSADKTTVQAVASA
IGRAIEDEARFGRIRDLEAKHFKKNVEEQLNKRVGHVYKKAFMQV
VEADMLSKGLLGGEAWSSWHKEDSIHVGVRCIEMLIESTGMVSLH
RQNAGVVGQDSETIELAPEYAEAIATRAGAMAGISPMFQPCVVPP
KPWTGITGGGYWANGRRPLALVRTRSKKALMHYEDVYMPEVYKAI
NIAQNTAWKINKKVLAVANVITKWKHCPVEDIPAIEREELPMKPE
DIDMNPEALTAWKRAAAAVYRKDKARKSRRISLEFMLEQANKFAN
HKAIWFPYNMDWRGRVYAVSMFNPQGNDMTKGLLTLAKGKPIGKE
GYYWLKIHGANCAGVDKVPFPERIKFIEENHENIMACAKSPLENT
WWAEQDSPFCFLAFCFEYAGVQHHGLSYNCSLPLAFDGSCSGIQH
FSAMLRDEVGGRAVNLLPSETVQDIYGIVAKKVNEILQADAINGT
DNEVVTVTDENTGEISEKVKLGTKALAGQWLAYGVTRSVTKRSVM
TLAYGSKEFGFRRQVLEDTIQPAIDSGKGLMFTQPNQAAGYMAKL
IWESVSVTVVAAVEAMNWLKSAAKLLAAEVKDKKTGEILRKRCAV
HWVTPDGFPVWQEYKKPIQTRLNLMFLGQFRLQPTINTNKDSEID
ARKQESGIAPNFVHSQDGSHLRKTVVWAHEKYGIESFALIHDSFG
TIPADAANLFKAVRETMVDTYESCDVLADFYDQFADQLHESQLDK
MPALPAKGNLNLRDILESDFAFA
Variant ES-444
SEQ ID NO: 19
MFLEPPKKKKKVVASLDNLVARYQRCFNDQSLKNSTIELEIRFQQ
INFLLFKTVYEALVAQEIPSTISHSIRCIKKVHHENHCREKILPS
ENLYFKKQPLMFFKFSEPASLGCKVSLAIEQPIRKFILDSSVLVR
LKNRTTFRVSELWKIELTIVKQLMSSEVSAKLAAFKTLLFDTPEQ
QTTRNMMTLINPDGEYLYEIEIEYTGKPESLTAADVIKIKNTVLT
LISPNHLMLTAYHQAIEFIASHILSSEILLARIKSGKWGLKRLLP
RVKSMTKANYMKFYPPVGYYVTDKADGIRGIAVIQDTQIYVVADQ
LYSLGTTGIEPLKPTILDGEFMPEKKEFYGFDVIMYEGNLLTQQG
FETRIESLSKGIKVLQAFDIKAEMKPFISLTSADPNVLLKNFESI
FKKKTRPYSIDGIILVEPGNSYLNTNTFKWKPTWDNTLDFLVRKC
PESLNVPEYAPKKGFSLHLLFVGISGELFKKLALNWCPGYTKLFP
VTQRNQNYFPVQFQPSDFPLAFLYYHPDTSSFSNIDGKVLEMRCL
KREINYVRWEIVRIREDRQQDLKTGGYFGNDFKTAELTWLNYMDP
FSFEELAKGPSGMYFAGAKTGIYRAQTALISFIKQEIIRKISHQS
WVIDLGIGKGQDLGRYLDAGVRHLVGIDKDQTALAELVYRKFSHA
TTRQHKHATNIYVLNQDLAEPAKEISEKVHQIYGFPKEGASSIVS
NLFIHYLMKNTQQVENLAVLCHKLLQPGGMVWFTTMLGEQVLELL
HENRIELNEVWEARENEVVKFAIKRLFKEDILQETGQEIGVLLPF
SNGDFYNEYLVNTAFLIKIFEHHGFSLVQKQSFKDWIPEFQNFSK
SLYKILTEADKTWTSLFGFICLRKNGGGGSGGGGSGGGGSLNTIN
IAKNDFSDIELAAIPFNTLADHYGERLAREQLALEHESYEMGEAR
FRKMFERQLKAGEVADNAAAKPLITTLLPKMIARINDWFEEVKAK
RGKRPTAFQFLQEIKPEAVAYITIKTTLACLTSADNTTVQAVASA
IGRAIEDEARFGRIRDLEAKHFKKDVEEQLNKRVGHVYKKAFMQV
VEADMLSKGLLGGEAWSSWHKEDSIHVGVRCIEMLIESTGMVSLH
RQNAGVVGQDSETIELAPEYAEAIATRAGALAGISPMFQPCVVPP
KPWTGITGGGYWANGRRPLALVRTRSKKALMHYEDVYMPEVYKAI
NIAQNTAWKINKKVLAVANVITKWKHCPVEDIPAIEREELPMKPE
DIDMNPEALTAWKRAAAAVYRKDKARKSRRISLEFMLEQANKFAN
HKAIWFPYNMDWRGRVYAVSMFNPQGNDMTKGLLTLAKGKPIGKE
GYYWLKIHGANCAGVDKVPFPERIKFIEENHENIMACAKFPLENT
WWAEQDSPFCFLAFCFEYAGVQHHGLSYNCSLPLAFDGSCSGIQH
FSAMLRDEVGGRAVNLLPSETVQDIYGIVAKKVNEILQADAINGT
DNEVVTVTDENTGEISEKVKLGTKALAGQWLAYGVTRSVTKRSVM
TLAYGSKEFGFRQQVLEDTIRPAIDSGKGLMFTQPNQAAGYMAKL
IWESVSVTVVAAVEAMNWLKSAAKLLAAEVKDKKTGEILRKRCAV
HWVTPDGFPVWQEYKKPIQTRLNLMFLGQFRLQPTINTNKDSEID
ARKQESGIAPNFVHSQDGSHLRKTVVWAHEKYGIESFALIHDSFG
TIPADAANLFKAVRETMVDTYESCDVLADFYDQFADQLHESQLDK
MPALPAKGNLNLRDILESDFAFA
SEQ ID NO: 20
Variant ES-446
MFLEPPKKKKKVVASLDNLVARYQRCFNDQSLKNSTIELEIRFQQ
INFLLFKTVYEALVAQEIPSTISHSIRCIKKVHHENHCREKILPS
ENLYFKKQPLMFFKFSEPASLGCKVSLAIEQPIRKFILDSSVLVR
LKNRTTFRVSELWKIELTIVKQLMSSEVSAKLAAFKTLLFDTPEQ
QTTRNMMTLINPDGEYLYEIEIEYTGKPESLTAADVIKIKNTVLT
LISPNHLMLTAYHQAIEFIASHILSSEILLARIKSGKWGLKRLLP
RVKSMTKANYMKFYPPVGYYVTDKADGIRGIAVIQDTQIYVVADQ
LYSLGTTGIEPLKPTILDGEFMPEKKEFYGFDVIMYEGNLLTQQG
FETRIESLSKGIKVLQAFDIKAEMKPFISLTSADPNVLLKNFESI
FKKKTRPYSIDGIILVEPGNSYLNTNTFKWKPTWDNTLDFLVRKC
PESLNVPEYAPKKGFSLHLLFVGISGELFKKLALNWCPGYTKLFP
VTQRNQNYFPVQFQPSDFPLAFLYYHPDTSSFSNIDGKVLEMRCL
KREINYVRWEIVKIREDRQQDLKTGGYFGNDFKTAELTWLNYMDP
FSFEELAKGPSGMYFAGAKTGIYRAQTALISFIKQEIIRKISHQS
WVIDLGIGKGQDLGRYLDAGVRHLVGIDKDQTALAELVYRKFSHA
TTRQHKHATNIYVLNQDLAEPAKEISEKVHQIYGFPKEGASSIVS
NLFIHYLMKNTQQVENLAVLCHKLLQPGGMVWFTTMLGEQVLELL
HENRIELNEVWEARENEVVKFAIKRLFKEDILQETGQEIGVLLPF
SNGDFYNEYLVNTAFLIKIFEHHGFSLVQKQSFKDWIPEFQNFSK
SLYKILTEADKTWTSLFGFICLRKNGGGGSGGGGSGGGGSLNTIN
IAKNDFSDIELAAIPFNTLADHYGERLAREQLALEHESYEMGEAR
FRKMFERQLKAGEVADNAAAKPLITTLLPKMIARINDWFEEVKAK
RGKRPTAFQFLQEIKPEAVAYITIKTTLACLTSADNTTVQAVASA
IGRAIEDEARFGRIRDLEAKHFKKDVEEQLNKRVGHVYKKAFMQV
VEADMLSKGLLGGEAWSSWHKEDSIHVGVRCIEMLIESTGMVSLH
RQNAGVVGQDSETIELAPEYAEAIATRAGALAGISPMFQPCVVPP
KPWTGITGGGYWANGRRPLALVRTRSKKALMHYEDVYMPEVYKAI
NIAQNTAWKINKKVLAVANVITKWKHCPVEDIPAIEREELPMKPE
DIDMNPEALTAWKRAAAAVYRKDKARKSRRISLEFMLEQANKFAN
HKAIWFPYNMDWRGRVYAVSMFNPQGNDMTKGLLTLAKGKPIGKE
GYYWLKIHGANCAGVDKVPFPERIKFIEENHENIMACAKFPLENT
WWAEQDSPFCFLAFCFEYAGVQHHGLSYNCSLPLAFDGSCSGIQH
FSAMLRDEVGGRAVNLLPSETVQDIYGIVAKKVNEILQADAINGT
DNEVVTVTDENTGEISEKVKLGTKALAGQWLAYGVTRSVTKRSVM
TLAYGSKEFGFRQQVLEDTIRPAIDSGKGLMFTQPNQAAGYMAKL
IWESVSVTVVAAVEAMNWLKSAAKLLAAEVKDKKTGEILRKRCAV
HWVTPDGFPVWQEYKKPIQTRLNLMFLGQFRLQPTINTNKDSEID
ARKQESGIAPNFVHSQDGSHLRKTVVWAHEKYGIESFALIHDSFG
TIPADAANLFKAVRETMVDTYESCDVLADFYDQFADQLHESQLDK
MPALPAKGNLNLRDILESDFAFA
Variant ES-447
SEQ ID NO: 21
MFLEPPKKNRKVVASLDNLVARYQRCFNDQSLKNSTIELEIRFQQ
INFLLFKTVYEALVAQEIPSTISHSIRCIKKVHHENHCREKILPS
ENLYFKKQPLMFFKFSEPASLGCKVSLAIEQPIRKFILDSSVLVR
LKNRTTFRVTELWKIELTIVKQLMSSEVSAKLAAFKTLLFDTPEQ
QTTKNMMTLINPDGEYLYEIEIEYTGKPESLTAADVIKIKNTVLT
LISPNHLMLTAYHQAIEFIASHILSSEILLARIKSGKWGLKRLLP
RVKSMTKANYMKFYPPVGYYVTDKADGIRGIAVIQDTQIYVVADQ
LYSLGTTGIEPLKPTILDGEFMPEKKEFYGFDVIMYEGNLLTQQG
FETRIESLSKGIKVLQAFNIKAEMKPFISLTSADPNVLLKNFESI
FKKKTRPYSIDGIILVEPGNSYLNTNTFKWKPTWDNTLDFLVRKC
PESLNVPEYAPKKGFSLHLLFVGISGELFKKLALNWCPGYTKLFP
VTQRNQNYFPVQFQPPDSPLAFLYYHPDTSSFSNIDGKVLEMRCL
KREINYVRWEIVRIREDRQQDLKTGGYFGNDFKTAELTWLNYMDP
FSFEELAKGPSGMYFAGAKTGIYRAQTALISFIKQEIIQKISHQS
WVIDLGIGKGQDLGRYLDAGVRHLVGIDKDQTALAELVYRKFSHA
TTRQHKHATNIYVLHQDLAEPAKEISEKVHQIYGFPKEGASSIVS
NLFIHYLMKNTQQVENLAVLCHKLLQPGGMVWFTTMLGEQVLELL
HENRIELNEVWEARENEVVKFAIKRLFKEDILQETGQEIGVLLPF
SNGDFYNEYLVNTAFLIKIFKHHGFSLVQKQSFKDWIPEFQNFSK
SLYKILTEADKTWTSLFGFICLRKNGGGGSGGGGSGGGGSLNTIN
IAKNDFSDIELAAIPFNTLADHYGERLAREQLALEHESYEMGEAR
FRKMFERQLKVGEVADNAAAKPLITTLLPKMIARINDWFEEVKAK
RGKRPTAFQFLQEIKPEAVAYITIKTTLACLTSADKTTVQAVASA
IGRAIEDEARFGRIRDLEAKHFRKNVEEQLNKRVGHVYKKAFMQV
VEADMLSKGLLGGEAWSSWHKEDSIHVGVRCIEMLIESTGMVSLH
RQNAGVVGQDSETIELAPEYAEAIATRAGALAGISPMFQPCVVPP
KPWTGITGGGYWANGRRPLALVRTRSKKALMHYEDVYMPEVYKAI
NIAQNTAWKINKKVLAVANVITKWKHCPVEDIPAIEREELPMKPE
DIDMNPEALTAWKRAAAAVYRKDKARKSRRISLEFMLEQANKFAN
HKAIWFPYNMDWRGRVYAVSMFNPQGNDMTKGLLTLAKGKPIGKE
GYYWLKIHGANCAGVDKVPFPERIKFIEENHENIMACAKSPLENT
WWAEQDSPFCFLAFCFEYAGVQHHGLSYNCSLPLAFDGSCSGIQH
FSAMLRDEVGGRAVNLLPSETVQDIYGIVAKKVNEILQADAINGT
DNEVVTVTDENTGEISEKVKLGTKALAGQWLAYGVTRSVTKRSVM
TLAYGSKEFGFRQQVLEDTIRPAIDSGKGLMFTQPNQAAGYMAKL
IWESVSVTVVAAVEAMNWLKSAAKLLAAEVKDKKTGEILRKRCAV
HWVTPDGFPVWQEYKKPIQTRLNLMFLGQFRLQPTINTNKDSEID
ARKQESGIAPNFVHSQDGSHLRKTVVWAHEKYGIESFALIHDSFG
TIPADAANLFKAVRETMVDTYESCDVLADFYDQFADQLHESQLDK
MPALPAKGNLNLRGILESDFAFA
Variant ES-448
SEQ ID NO: 22
MFLEPPKKNRKVVASLDNLVARYQRCFNDQSLKNSTIELEIRFQQ
INFLLFKTVYEALVAQEIPSTISHSIRCIKKVHHENHCREKILPS
ENLYFKKQPLMFFKFSEPASLGCKVSLAIEQPIRKFILDSSVLVR
LKNRTTFRVSELWKIELTIVKQLMGSEVSAKLAAFKTLLFDTPEQ
QTTKNMMTLINPDGEYLYEIEIEYTGKPESLTAADVIKIKNTVLT
LISPNHLMLTAYHQAIEFIASHILSSEILLARIKSGKWGLKRLLP
RVKSMTKANYMKFYPPVGYYVTDKADGIRGIAVIQDTQIYVVADQ
LYSLGTTGIEPLKPTILDGEFMPEKKEFYGFDVIMCEGNLLTQQG
FETRIESLSKGIKVLQAFNIKAEMKPFISLTSADPNVLLKNFESI
FKKKTRPYSIDGIILVEPGNSYLNTNTFKWKPTWDNTLDFLVRKC
PESLNVPEYAPKKGFSLHLLFVGISGELFKKLALNWCPGYTKLFP
VTQRNQNYFPVQFQPSDFPLAFLYYHPDTSSFSNIDGKVLEMRCL
KREINYVRWEIVKIREDRQQDLKTGGYFGNDFKTAELTWLNYMDP
FSFEELAKGPSGMYFAGAKTGIYRAQTALISFIKQEIIRKISHQS
WVIDLGIGKGQDLGRYLDAGVRHLVGIDKDQTALAELVYRKFSHA
TTRQHKHATNIYVLHQDLAEPAKEISEKVHQIYGFPKEGASSIVS
NLFIHYLMKNTQQVENLAVMCHKLLQPGGMVWFTTMLGEQVLELL
HENRIELNEVWEARENEVVKFAIKRLFKEDILQETGQEIGVLLPF
SNGDFYNEYLVNTAFLIKIFKHHGFSLVQKQSFKDWIPEFQNFSK
SLYKILTEADKTWTSLFGFICLRKNGGGGSGGGGSGGGGSLNTIN
IAKNDFSDIELAAIPFNTLADHYGERLAREQLALEHESYEMGEAR
FRKMFERQLKAGEVADNAAAKPLITTLLPKMIARINNWFEEVKAK
RGKRPTAFQFLQEIKPEAVAYITIKTTLACLTSADKTTVQAVASA
IGRAIEDEARFGRIRDLEAKHFKKNVEEQLNKRVGHVYKKAFMQV
VEADMLSKGLLGGEAWSSWHKEDSIHVGVRCIEMLIESTGMVSLH
RQNAGVVGQDSETIELAPEYAEAIATRAGAMAGISPMFQPCVVPP
KPWTGITGGGYWANGRRPLALVRTHSKKALMRYEDVYMPEVYKAI
NIAQNTAWKINEKVLAVANVITKWKHCPVEDIPAIEREELPMKPE
DIDMNPEALTAWKRAAAAVYRKDKARKSRRISLEFMLEQANKFAN
HKAIWFPYNMDWRGRVYAVSMFNPQGNDMTKGLLTLAKGKPIGKE
GYYWLKIHGANCAGVDKVPFPERIKFIEENHENIMACAKSPLENT
WWAEQDSPFCFLAFCFEYAGVQHHGLSYNCSLPLAFDGSCSGIQH
FSAMLRDEVGGRAVNLLPSETVQDIYGIVAKKVNEILQADAINGT
DNEVVTVTDENTGEISEKVKLGTKALAGQWLAYGVTRSVTKRSVM
TLAYGSKEFGFRQQVLEDTIQPAIDSGKGLMFTQPNQAAGYMAKL
IWESVNVTVVAAVEAMNWLKSAAKLLAAEVKDKKTGEILRKRCAV
HWVTPDGFPVWQEYKKPIQTRLNLMFLGQFRLQPTINTNKDSEID
ARKQESGIAPNFVHSQDGSHLRKTVVWAHEKYGIESFALIHDSFG
TIPADAANLFKAVRETMVDTYESCDVLADFYDQFADQLHESQLDK
MPALPAKGNLNLRDILESDFAFA
Variant ES-45:
SEQ ID NO: 23
MFLEPPKKNRKVVASLDNLVARYQRCFNDQSLKNSTIELEIRFQQ
INFLLFKTVYEALVAQEIPSTISHSIRCIKKVHHENHCREKILPS
ENLYFKKQPLMFFKFSEPASLGCKVSLAIEQPIRKFILDSSVLVR
LKNRTTFRVSELWKIELTIVKQLMGSEVSAKLAAFKTLLFDTPEQ
QTTKNMMTLINPDGEYLYEIEIEYTGKPESLTAADVIKIKNTVLT
LISPNHLMLTAYHQAIEFIASHILSSEILLARIKSGKWGLKRLLP
RVKSMTKADYMKFYPPVGYYVTDKADGIRGIAVIQDTQIYVVADQ
LYSLGTTGIEPLKPTILDGEFMPEKKEFYGFDVIMYEGNLLTQQG
FETRIESLSKGIKVLQAFNIKAEMKPFISLTSADPNVLLKNFESI
FKKKTRPYSIDGIILVEPGNSYLNTNTFKWKPTWDNTLDFLVRKC
PESLNVPEYAPKKGFSLHLLFVGISGELFKKLALNWCPGYTKLFP
VTQRNQNYFPVQFQPSDFPLAFLYYHPDTSSFSNIDGKVLEMRCL
KREINYVRWEIVKIREDRQQDLKTGGYFGNDFKTAELTWLNYMDP
FSFEELAKGPSGTYFAGAKTGIYRAQTALISFIKQEIIQKISHQS
WVIDLGIGKGQDLGRYLDAGVRHLVGIDKDQTALAELVYRKFSHA
TTRQHKHATNIYVLHQDLAEPAKEISEKVHQIYGFPKEGASSIVS
NLFIHYLMKNTQQVENLAVLCHKLLQPGGMVWFTTMLGEQVLELL
HENRIELNEVWEARENEVVKFAIKRLFKEDILQETGQEIGVLLPF
SNGDFYNEYLVNTAFLIKIFKHHGFSLVQKQSFKDWIPEFQNFSK
SLYKILTEADKTWTSLFGFICLRKNGGGGSGGGGSGGGGSLNTIN
IAKNDFSDIELAAIPFNTLADHYGERLAREQLALEHESYEMGEAR
FRKMFERQLKAGEVADNAAAKPLITTLLPKMIARINNWFEEVKAK
RGKRPTAFQFLQEIKPEAVAYITIKTTLACLTSADNTTVQAVASA
IGRAIEDEARFGRIRDLEAKHFKKNVEEQLNKRVGHVYKKAFMQV
VEADMLSKGLLGGEAWPSWHKEDSIHVGVRCIEMLIESTGMVSLH
RQNAGVVCQDSETIELAPEYAEAIATRAGALAGISPMFQPCVVPP
KPWTGITGGGYWANGRRPLALVRTRSKKALMHYEDVYMPEVYKAI
NIAQNTAWKINKKVLAVANVITKWKHCPVEDIPAIEREELPMKPE
DIDMNPEALTAWKRAAAAVYRKDKARKSRRISLEFMLEQANKFAN
HKAIWFPYNMDWRGRVYAVSMFNPQGNDMTKGLLTLAKGKPIGKE
GYYWLKIHGANCAGVDKVPFPERIKFIEENHENIMACAKSPLENT
WWAEQDSPFCFLAFCFEYAGVQHHGLSYNCSLPLAFDGSCSGIQH
FSAMLRDEVGGRAVNLLPSETVQDIYGIVAKKVNEILQADAINGT
DNEVVTVTDENTGEISEKVKLGTKALAGQWLAYGVTRSVTKRSVM
TLAYGSKEFGFRQQVLEDTIQPAIDSGKGLMFTQPNQAAGYMAKL
IWESVNVTVVAAVEAMNWLKSAAKLLAAEVKDKKTGEILRKRCAV
HWVTPDGFPVWQEYKKPIQTRLNLMFLGQFRLQPTINTNKDSEID
ARKQESGIAPNFVHSQDGSHLRKTVVWAHEKYGIESFALIHDSFG
TIPADAANLFKAVRETMVDTYESCDVLADFYDQFADQLHESQLDK
MPALPAKGNLNLRDILESDFAFA
Variant ES-451
SEQ ID NO: 24
MFLEPPKKKIKVVASLDNLVARYQRCFNDQSLKNSTIELEIRFQQ
INFLLFKTVYEALVAQEIPSTISHSIRCIKKVHHENHCREKILPS
ENLYFKKQPLMFFKFSEPASLGCKVSLAIEQPIRKFILDSSVLVR
LKNRTTFRVSELWKIELTIVKQLMGSEVSAKLAAFKTLLFDTPEQ
QTTKNMMTLINPDGEYLYEIEIEYTGKPESLTAADVIKIKNTVLT
LISPNHLMLTAYHQAIEFIASHILSSEILLARIKSGKWGLKRLLP
RVKSMTKANYMKFYPPVGYYVTDKADGIRGIAVIQDTQIYVVADQ
LYSLGTTGIEPLKPTILDGEFMPEKKEFYGFDVIMYEGNLLTQQG
FETRIESLSKGIKVLQAFDIKAEMKPFISLTSADPNVLLKNFESI
FKKKTRPYSIDGIILVEPGNSYLNTNTFKWKPTWDNTLDFLVRKC
PESLNVPEYAPKKGFSLHLLFVGISGELFKKLALNWCPGYTKLFP
VTQRNQNYFPVQFQPSDFPLAFLYYHPDTSSFSNIDGKVLEMRCL
KREINYVRWEIVKIREDRQQDLKTGGYFGNDFKTAELTWLNYMDP
FSFEELAKGPSGMYFAGAKTGIYRAQTALISFIKQEIIQKISHQS
WVIDLGIGKGQDLGRYLDAGVRHLVGIDKDQTALAELVYRKFSHA
TTRQHKHATNIYVLHQDLAEPAKEISEKVHQIYGFPKEGASSIVS
NLFIHYLMKNTQQVENLAVMCHKLLQPGGMVWLTTMLGEQVLELL
HENRIELNEVWEARENEVVKFAIKRLFKEDILQETGQEIGVLLPF
SNGDFYNEYLVNTAFLIKIFKHHGFSLVQKQSFKDWIPEFQNFSK
SLYKILTEADKTWTSLFGFICLRKNGGGGSGGGGSLNTINIAKND
FSDIELAAIPFNTLADHYGERLAREQLALEHESYEMGEARFRKMF
ERQLKAGEVADNAAAKPLITTLLPKMIARINDRFEEVKAKRGKRP
TAFQFLQEIKPEAVAYITIKTTLACLTSADNTTVQAVASAIGRAI
EDEARFGRIRDLEAKHFKKNVEEQLNKRVGHVYKKAFMQVVEADM
LSKGLLGGEAWSSWHKEDSIHVGVRCIEMLIESTGMVSLHRQNAG
VVGQDSETIELAPEYAEAIATRAGAMAGISPMFQPCVVPPKPWTG
ITGGGYWANGRRPLALVRTRSKKALMHYEDVYMPEVYKAINIAQN
TAWKINKKVLAVANVITKWKHCPVEDIPAIEREELPMKPEDIDMN
PEALTAWKRAAAAVYRKDKARKSRRISLEFMLEQANKFANHKAIW
FPYNMDWRGRVYAVSMFNPQGNDMTKGLLTLAKGKPIGKEGYYWL
KIHGANCAGVDKVPFPERIKFIEENHENIMACAKSPLENTWWAEQ
DSPFCFLAFCFEYAGVQHHGLSYNCSLPLAFDGSCSGIQHFSAML
RDEVGGRAVNLLPSETVQDIYGIVAKKVNEILQADAINGTDNEVV
TVTDENTGEISEKVKLGTKALAGQWLAYGVTRSVTKRSVMTLAYG
SKEFGFRRQVLEDTIQPAIDSGKGLMFTQPNQAAGYMAKLIWESV
NVTVVAAVEAMNWLKSAAKLLAAEVKDKKTGEILRKRCAVHWVTP
DGFPVWQEYKKPIQTRLNLMFLGQFRLQPTINTNKDSEIDARKQE
SGIAPNFVHSQDGSHLRKTVVWAHEKYGIESFALIHDSFGTIPAD
AANLFKAVRETMVDTYESCDVLADFYDQFADQLHESQLDKMPALP
AKGNLNLRDILESDFAFA
SEQ ID NO: 25
TAATACGACTCACTATA
SEQ ID NO 26
TAATACGACTCACTAAA
SEQ ID NO 27
TAATACGACTCACTGTA
SEQ ID NO 28
TAATACGACTCACTCTA
SEQ ID NO: 29
TAATACCGGTCACTATA
SEQ ID NO: 30
TAATACCTGACACTATA
SEQ ID NO: 31
TAATAACCCTCACTATA
SEQ ID NO: 32
TAATAACTATCACTATA
SEQ ID NO: 33
TAATAACCCACACTATA
WT-NPT7
SEQ ID NO: 34
MGHHHHHHHHHHGSSAWSHPQFEKGGGSGGGSGGGSWSHPQFEKG
SLEVLFQGPGSFFEPPKKKRKVVASLDNLVARYQRCFNDQSLKNS
TIELEIRFQQINFLLFKTVYEALVAQEIPSTISHSIRCIKKVHHE
NHCREKILPSENLYFKKQPLMFFKFSEPASLGCKVSLAIEQPIRK
FILDSSVLVRLKNRTTFRVSELWKIELTIVKQLMGSEVSAKLAAF
KTLLFDTPEQQTTKNMMTLINPDGEYLYEIEIEYTGKPESLTAAD
VIKIKNTVLTLISPNHLMLTAYHQAIEFIASHILSSEILLARIKS
GKWGLKRLLPRVKSMTKADYMKFYPPVGYYVTDKADGIRGIAVIQ
DTQIYVVADQLYSLGTTGIEPLKPTILDGEFMPEKKEFYGFDVIM
YEGNLLTQQGFETRIESLSKGIKVLQAFNIKAEMKPFISLTSADP
NVLLKNFESIFKKKTRPYSIDGIILVEPGNSYLNTNTFKWKPTWD
NTLDFLVRKCPESLNVPEYAPKKGFSLHLLFVGISGELFKKLALN
WCPGYTKLFPVTQRNQNYFPVQFQPSDFPLAFLYYHPDTSSFSNI
DGKVLEMRCLKREINYVRWEIVKIREDRQQDLKTGGYFGNDFKTA
ELTWLNYMDPFSFEELAKGPSGMYFAGAKTGIYRAQTALISFIKQ
EIIQKISHQSWVIDLGIGKGQDLGRYLDAGVRHLVGIDKDQTALA
ELVYRKFSHATTRQHKHATNIYVLHQDLAEPAKEISEKVHQIYGF
PKEGASSIVSNLFIHYLMKNTQQVENLAVLCHKLLQPGGMVWFTT
MLGEQVLELLHENRIELNEVWEARENEVVKFAIKRLFKEDILQET
GQEIGVLLPFSNGDFYNEYLVNTAFLIKIFKHHGFSLVQKQSFKD
WIPEFQNFSKSLYKILTEADKTWTSLFGFICLRKNGGGGSGGGGS
GGGGSLNTINIAKNDFSDIELAAIPFNTLADHYGERLAREQLALE
HESYEMGEARFRKMFERQLKAGEVADNAAAKPLITTLLPKMIARI
NDWFEEVKAKRGKRPTAFQFLQEIKPEAVAYITIKTTLACLTSAD
NTTVQAVASAIGRAIEDEARFGRIRDLEAKHFKKNVEEQLNKRVG
HVYKKAFMQVVEADMLSKGLLGGEAWSSWHKEDSIHVGVRCIEML
IESTGMVSLHRQNAGVVGQDSETIELAPEYAEAIATRAGALAGIS
PMFQPCVVPPKPWTGITGGGYWANGRRPLALVRTHSKKALMRYED
VYMPEVYKAINIAQNTAWKINKKVLAVANVITKWKHCPVEDIPAI
EREELPMKPEDIDMNPEALTAWKRAAAAVYRKDKARKSRRISLEF
MLEQANKFANHKAIWFPYNMDWRGRVYAVSMFNPQGNDMTKGLLT
LAKGKPIGKEGYYWLKIHGANCAGVDKVPFPERIKFIEENHENIM
ACAKSPLENTWWAEQDSPFCFLAFCFEYAGVQHHGLSYNCSLPLA
FDGSCSGIQHFSAMLRDEVGGRAVNLLPSETVQDIYGIVAKKVNE
ILQADAINGTDNEVVTVTDENTGEISEKVKLGTKALAGQWLAYGV
TRSVTKRSVMTLAYGSKEFGFRQQVLEDTIQPAIDSGKGLMFTQP
NQAAGYMAKLIWESVSVTVVAAVEAMNWLKSAAKLLAAEVKDKKT
GEILRKRCAVHWVTPDGFPVWQEYKKPIQTRLNLMFLGQFRLQPT
INTNKDSEIDAHKQESGIAPNFVHSQDGSHLRKTVVWAHEKYGIE
SFALIHDSFGTIPADAANLFKAVRETMVDTYESCDVLADFYDQFA
DQLHESQLDKMPALPAKGNLNLRDILESDFAFA

1. An engineered enzyme comprising a T7 RNA polymerase component and a capping enzyme component separated by a linker, wherein the T7 RNA polymerase component comprises 90% or more identity to SEQ ID NO: 3 and the capping enzyme component comprises 90% or more identity to SEQ ID NO: 5.

2. The engineered enzyme of claim 1, wherein the engineered enzyme comprises at least one substitution which confers at least one improved property compared to the engineered enzyme without the substitution.

3. The engineered enzyme of claim 1, wherein the linker can vary in length or amino acid composition.

4. An engineered enzyme comprising SEQ ID NO: 1 with at least one substitution which confers at least one improved property compared to SEQ ID NO: 1 without the substitution, and further wherein positions 881-896 of the engineered enzyme comprise a linker which can vary in length or amino acid composition.

5. The engineered enzyme of claim 4, wherein a substitution exists at one or more of the following positions of SEQ ID NO: 1: D279, H1667, K9, H1195, N379, Q624, L740, K1058, L1156, R1202, Q1543, S1581, D1769.

6-30. (canceled)

31. The engineered enzyme of claim 4, wherein a substitution exists at one or more of the following positions of SEQ ID NO: 1: R10, G160, K553, H690, F753, K831, D921, W983, 11012, A1024, N1026, G1133, and/or S1390.

32-44. (canceled)

45. The engineered enzyme of any one of claim 4, wherein a substitution exists at one or more of the following positions of SEQ ID NO: 1: K184, Q624, D982, N1060, and/or Q1551.

46-50. (canceled)

51. The engineered enzyme of claim 4, wherein a substitution exists at one or more of the following positions of SEQ ID NO: 1: R22, Q30, E38, Q45, Q45, Q98, L100, R124, R143, M159, 1324, N396, T497, Q498, N502, M598, R678, Q679, Q760, H832, L911, 1914, H922, R952, R994, T1022, D1025, T1027, Y1073, L1090, L1091, W1096, H1100, V1131, F1163, W1239, M1257, M1264, M1296, I1476, N1487, I1500, F1539, M1561, C1618, L1647, 11705, V1723, M1727, and/or C1734.

52. (canceled)

53. The engineered enzyme of claim 51, wherein said engineered enzyme comprises at least one improved property compared to SEQ ID NO: 1.

54. The engineered enzyme of claim 4, wherein the at least one improved property is selected from improved selectivity for capping, improved processivity of capping, improved capping enzymatic activities, improved protein expression, improved RNA yield, improved stability in storage buffer, improved stability under reaction conditions, improved processivity of translation, improved thermostability, and improved transcription fidelity.

55. The engineered enzyme of claim 54, wherein the improved enzymatic activities comprise improvement to activity of RNA triphosphatase guanyltransferase, and/or methyltransferase.

56. A nucleic acid encoding the engineered enzyme of claim 1.

57. The nucleic acid of claim 56, wherein said polynucleotide sequence is operably linked to a control sequence.

58. An expression vector comprising the nucleic acid sequence of claim 55.

59. A host cell comprising the expression vector of claim 58.

60-80. (canceled)

81. A method of selecting one or more engineered enzymes comprising a non-eukaryotic polymerase component and a capping enzyme component, wherein the engineered enzyme comprises enhanced activity compared to a control, the method comprising:
a) Creating a nucleic acid encoding the one or more engineered enzyme variants, wherein said variants comprise a variant of a naturally occurring non-eukaryotic polymerase and/or a variant of a naturally occurring capping enzyme component;
b) Integrating said nucleic acid encoding one or more engineered enzyme variants into a one or more eukaryotic cells, wherein said eukaryotic cells comprises a reporter, wherein said reporter is under the control of a polymerase promoter which is specific for the polymerase of the engineered enzyme, and further wherein the reporter is only expressed when it is capped by said capping enzyme;
c) Expressing said nucleic acid encoding one or more engineered enzyme variants; and
d) Determining which of the one or more variants confer enhanced activity compared to a control and selecting said engineered enzyme variant.

82. The method of claim 81, wherein said naturally occurring non-eukaryotic polymerase and naturally occurring capping enzyme component are not naturally occurring in the same organism.

83. The method of claim 81, wherein the polymerase is T7 RNA polymerase.

84. The method of claim 81, wherein the capping enzyme is NP868R.

85. The method of claim 81, wherein said polymerase and capping enzyme are separated by a linker.

86-113. (canceled)


SEQUENCE LISTING (34 sequences)

SEQ ID NO: 1 - protein, 1778 aa, synthetic construct
MFLEPPKKKRKVVASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVSELWKIELTIVKQLMGSEVSAKLAAFKTLLFDTPEQQTTKNMMTLINPDGEYLYEIEIEYTGKPESLTAADVIKIKNTVLTLISPNHLMLTAYHQAIEFIASHILSSEILLARIKSGKWGLKRLLPRVKSMTKADYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFNIKAEMKPFISLTSADPNVLLKNFESIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINYVRWEIVKIREDRQQDLKTGGYFGNDFKTAELTWLNYMDPFSFEELAKGPSGMYFAGAKTGIYRAQTALISFIKQEIIQKISHQSWVIDLGIGKGQDLGRYLDAGVRHLVGIDKDQTALAELVYRKFSHATTRQHKHATNIYVLHQDLAEPAKEISEKVHQIYGFPKEGASSIVSNLFIHYLMKNTQQVENLAVLCHKLLQPGGMVWFTTMLGEQVLELLHENRIELNEVWEARENEVVKFAIKRLFKEDILQETGQEIGVLLPFSNGDFYNEYLVNTAFLIKIFKHHGFSLVQKQSFKDWIPEFQNFSKSLYKILTEADKTWTSLFGFICLRKNGGGGSGGGGSGGGGSLNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 2 - protein, 13 aa, synthetic construct
MFLEPPKKKRKVV

SEQ ID NO: 3 - protein, 882 aa, synthetic construct
NTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 4 - protein, 16 aa, synthetic construct
GGGGSGGGGSGGGGSL

SEQ ID NO: 5 - protein, 867 aa, synthetic construct
ASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVSELWKIELTIVKQLMGSEVSAKLAAFKTLLFDTPEQQTTKNMMTLINPDGEYLYEIEIEYTGKPESLTAADVIKIKNTVLTLISPNHLMLTAYHQAIEFIASHILSSEILLARIKSGKWGLKRLLPRVKSMTKADYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFNIKAEMKPFISLTSADPNVLLKNFESIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINYVRWEIVKIREDRQQDLKTGGYFGNDFKTAELTWLNYMDPFSFEELAKGPSGMYFAGAKTGIYRAQTALISFIKQEIIQKISHQSWVIDLGIGKGQDLGRYLDAGVRHLVGIDKDQTALAELVYRKFSHATTRQHKHATNIYVLHQDLAEPAKEISEKVHQIYGFPKEGASSIVSNLFIHYLMKNTQQVENLAVLCHKLLQPGGMVWFTTMLGEQVLELLHENRIELNEVWEARENEVVKFAIKRLFKEDILQETGQEIGVLLPFSNGDFYNEYLVNTAFLIKIFKHHGFSLVQKQSFKDWIPEFQNFSKSLYKILTEADKTWTSLFGFICLRKN

SEQ ID NO: 6 - protein, 1778 aa, synthetic construct
MFLEPPKKKIKVVASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVSELWKIELTIVKQLMSSEVSAKLAAFKTLLFDTPEQQTTKNMMTLINPDGEYLYEIEIEYTGKPESLTAADVIKIKNTVLTLISPNHLMLTAYHQAIEFIASHILSSEILLARIKSGKWGLKRLLPRVKSMTKADYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFNIKAEMKPFISLTSADPNVLLKNFESIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINYVRWEIVKIREDRQQDLKTGGYFGNDFKTAELTWLNYMDPFSFEELAKGPSGMYFAGAKTGIYRAQTALISFIKQEIIQKISHQSWVIDLGIGKGQDLGRYLDAGVRHLVGIDKDQTALAELVYRKFSHATTRQHKHATNIYVLHQDLAEPAKEISEKVHQIYGFPKEGASSIVSNLFIHYLMKNTQQVENLAVLCHKLLQPGGMVWFTTMLGEQVLELLHENRIELNEVWEARENEVVKFAIKRLFKEDILQETGQEIGVLLPFSNGDFYNEYLVNTAFLIKIFEHHGFSLVQKQSFKDWIPEFQNFSKSLYKILTEADKTWTSLFGFICLRKSGGGGSGGGGSGGGGSLNTINIAKNDFSDIELAAIPFNTLADHYGERLAREQLALEHESYEMGEARFRKMFERQLKAGEVADNAAAKPLITTLLPKMIARINNWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITCGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRRQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDARKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 7 - protein, 1778 aa, synthetic construct
MFLEPPKKKKKVVASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVSELWKIELTIVKQLMGSEVSAKLAAFKTLLFDTPEQQTTKNMMTLINPDGEYLYEIEIEYTGKPESLTAADVIKIKNTVLTLISPNHLMLTAYHQAIEFIASHILSSEILLARIKSGKWGLKRLLPRVKSMTKAYYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFNIKAEMKPFISLTSADPNVLLKNFESIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINYVRWEIVKIREDRQQDLKTGGYFGNDFKTAELTWLNYMDPFSFEELAKGPSGMYFAGAKTGIYRAQTALISFIKREIIQKISHQSWVIDLGIGKGQDLGRYLDAGVRHLVGIDKDQTALAELVYRKFSHATTRQHKHATNIYVLHQDLAEPAKEISEKVHQIYGFPKEGASSIVSNLFIHYLMKNTQQVENLAVMCHKLLQPGGMVWLTTMLGEQVLELLHENRIELNEVWEARENEVVKFAIKRLFKEDILQETGQEIGVLLPFSNGDFYNEYLVNTAFLIKIFKHHGFSLVQKQSFKDWIPEFQNFSKSLYKILTEADKTWTSLFGFICLRKNGGGGSGGGGSGGGGSLNTINIAKNDFSDIELAAIPFNTLADHYGERLAREQLALEHESYEMGEARFRKMFERQLKAGEVADNAAAKPLITTLLPKMIARINDRFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGAMAGISPMFQPCVVPPKPWTGITGGGYWANGRRPLALVRTHSKKALMRYEDVYMPEVYKAINIAQNTAWKINEKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKFPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVNVTVVAAVEAMNWLKSAAKLLAAEVKDKKTGEILRKRCAVHWVTPDGFPVWQEYKKPIQTRLNLMFLGQFRLQPTINTNKDSEIDARKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRGILESDFAFA

SEQ ID NO: 8 - protein, 1778 aa, synthetic construct
MFLEPPKKNRKVVASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVSELWKIELTIVKQLMGSEVSAKLAAFKTLLFDTPEQQTTKNMMTLINPDGEYLYEIEIEYTGKPESLTAADVIKIKNTVLTLISPNHLMLTAYHQAIEFIASHILSSEILLARIKSGKWGLKRLLPRVKSMTKANYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFDIKAEMKPFISLTSADPNVLLKNFESIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINYVRWEIVRIREDRQQDLKTGGYFGNDFKTAELTWLNYMDPFSFEELAKGPSGMYFAGAKTGIYRAQTALISFIKQEIIRKISHQSWVIDLGIGKGQDLGRYLDAGVRHLVGIDKDQTALAELVYRKFSHATTRQHKHATNIYVLHQDLAEPAKEISEKVHQIYGFPKEGASSIVSNLFIHYLMKNTQQVENLAVLCHKLLQPGGMVWFTTMLGEQVLELLHENRIELNEVWEARENEVVKFAIKRLFKEDILQETGQEIGVLLPFSNGDFYNEYLVNTAFLIKIFKHHGFSLVQKQSFKDWIPEFQNFSKSLYKILTEADKTWTSLFGFICLRKNGGGGSGGGGSGGGGSLNTINIAKNDFSDIELAAIPFNTLAGHYGERLAREQLALEHESYEMGEARFRKMFERQLKAGEVADNAAAKPLITTLLPKMIARINDWFEEVKAKRGKRPTAFQFLQEIKPEAVAYTTIKTTLACLTSTDNTTVQAVASAIGRAIEDEARFGRIRDLEAKHFRKNVEEQLNKRVGHVYKKAFMQVVEADMLSKGLLGGEAWSSWHKEDSIHVGVRCIEMLIESTGMVSLHRQNAGVVCQDSETIELAPEYAEAIATRAGALAGISPMFQPCVVPPKPWTGITGGGYWANGRRPLALVRTRSKKALMHYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQGSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIRPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDARKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRGILESDFAFA

SEQ ID NO: 9 - protein, 1778 aa, synthetic construct
MFLEPPKKNRKVVASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVSELWKIELTIVKQLMGSEVSAKLAAFKTLLFDTPEQQTTKNMMTLINPDGEYLYEIEIEYTGKPESLTAADVIKIKNTVLTLISPNHLMLTAYHQATEFIASHILSSEILLARIKSGKWGLKRLLPRVKSMTKANYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFDIKAEMKPFISLTSADPNVLLKNFESIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINYVRWEIVRIREDRQQDLKTGGYFGNDFKTAELTWLNYMDPFSFEELAKGPSGMYFAGAKTGIYRAQTALISFIKQEIIQKISHQSWVIDLGIGKGQDLGRYLDAGVRHLVGIDKDQTALAELVYRKFSHATTRQHKHATNIYVLHQDLAEPAKEISEKVHQIYGFPKEGASSIVSNLFIHYLMKNTQQVENLAVLCHKLLQPGGMVWFTTMLGEQVLELLHENRIELNEVWEARENEVVKFAIKRLFKEDILQETGQEIGVLLPFSNGDFYNEYLVNTAFLIKIFKHHGFSLVQKQSFKDWIPEFQNFSKSLYKILTEADKTWTSLFGFICLRKNGGGGSGGGGSGGGGSLNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFRKNVEEQLNKRVGHVYKKAFMQVVEADMLSKGLLGGEAWSSWHKEDSIHVGVRCIEMLIESTGMVSLHRQNAGVVGQDSETIELAPEYAEAIETRAGALAGISPMFQPCVVPPKPWTGITGGGYWANGRRPLALVRTRSKKALMHYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIRPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDARKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRGILESDFAFA

SEQ ID NO: 10 - protein, 1778 aa, synthetic construct
MFLEPPKKNRKVVASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVSELWKIELTIVKQLMGSEVSAKLAAFKTLLFDTPEQQTTKNMMTLINPDGEYLYEIEIEYTGKPESLTAADVIKIKNTVLTLISPNHLMLTAYHQAIEFIASHILSSEILLARIKSGKWGLKRLLPRVKSMTKANYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFNIKAEMKPFISLTSADPNVLLKNFESIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINYVRWEIVKIREDRQQDLKTGGYFGNDFKTAELTWLNYMDPFSFEELAKGPSGMYFAGAKTGIYRAQTALISFIKQEIIRKISHQSWVIDLGIGKGQDLGRYLDAGVRHLVGIDKDQTALAELVYRKFSHATTRQHKHATNIYVLHQDLAEPAKEISEKVHQIYGFPKEGASSIVSNLFIHYLMKNTQQVENLAVMCHKLLQPGGMVWFTTMLGEQVLELLHENRIELNEVWEARENEVVKFAIKRLFKEDILQETGQEIGVLLPFSNGDFYNEYLVNTAFLIKIFKHHGFSLVQKQSFKDWIPEFQNFSKSLYKILTEADKTWTSLFGFICLRKNGGGGSGGGGSGGGGSLNTINIAKNDSSDIELAAIPFNTLADHYGERLAREQLALEHESYEMGEARFRKMFERQLKAGEVADNAAAKPLITTLLPKMIARINDRFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGAMAGISPMFQPCVVPPKPWTGITGGGYWANGRRPLALVRTRSKKALMHYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVNVTVVAAVEAMNWLKSAAKLLAAEVKDKKTGEILRKRCAVHWVTPDGFPVWQEYKKPIQTRLNLMFLGQFRLQPTINTNKDSEIDARKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 11 - protein, 1778 aa, synthetic construct
MFLEPPKKNRKVVASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVSELWKIELTIVKQLMGSEVSAKLAAFKTLLFDTPEQQTTKNMMTLINPDGEYLYEIEIEYTGKPESLTAADVIKIKNTVLTLISPNHLMLTAYHQAIEFIASHILSSEILLARIKSGKWGLKRLLPRVKSMTKANYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFDIKAEMKPFISLTSADPNVLLKNFEFIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINYVRWEIVRIREDRQQDLKTGGYFGNDFKTAELTWLNYMDPFSFEELAKGPSGMYFAGAKTGIYRAQTALISFIKQEIIRKISHQSWVIDLGIGKGQDLGRYLDAGVRHLVGIDKDQTALAELVYRKFSHATTRQHKHATNIYVLHQDLAEPAKEISEKVHQIYGFPKEGASSIVSNLFIHYLMKNTQQVENLAVLCHKLLQPGGMVWFTTMLGEQVLELLHENRIELNEVWEARENEVVKFAIKRLFKEDILQETGQEIGVLLPFSNGDFYNEYLVNTAFLIKIFKHHGFSLVQKQSFKDWIPEFQNFSKSLYKILTEADKTWTSLFGFICLRKNGGGGSGGGGSGGGGSLNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYTTIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVCQDSETIELAPEYAEAIATRAGALAGISPMFQPCVVPPKPWTGITGGGYWANGRRPLALVRTRSKKALMHYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIRPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDARKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRGILESDFAFA

SEQ ID NO: 12 - protein, 1778 aa, synthetic construct
MFLEPPKKNRKVVASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVSELWKIELTIVKQLMGSEVSAKLAAFKTLLFDTPEQQTTKNMMTLINPDGEYLYEIEIEYTGKPESLTAADVIKIKNTVLTLISPNHLMLTAYHQAIEFIASHILSSEILLARIKSGKWGLKRLLPRVKSMTKANYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFNIKAEMKPFISLTSADPNVLLKNFESIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINYVRWETVKIREDRQQDLKTGGYFGNDFKTAELTWLNYMDPFSFEELAKGPSGMYFAGAKTGIYRAQTALISFIKREIIRKISHQSWVIDLGIGKGQDLGRYLDAGVRHLVGIDKDQTALAELVYRKFSHATTRQHKHATNIYVLHQDLAEPAKEISEKVHQIYGFPKEGASSIVSNLFIHYLMKNTQQVENLAVMCHKLLQPGGMVWLTTMLGEQVLELLHENRIELNEVWEARENEVVKFAIKRLFKEDILQETGQEIGVLLPFSNGDFYNEYLVNTAFLIKIFKHHGFSLVQKQSFKDWIPEFQNFSKSLYKILTEADKTWTSLFGFICLRKNGGGGSGGGGSGGGGSLNTINIAKNDFSDIELAAIPFNTLAGHYGERLAREQLALEHESYEMGEARFRKMFERQLKAGEVADNAAAKPLITTLLPKMIARINDRFEEVKAKRGKRPTAFQFLQEIKPEAVAYITIKTTLACLTSADNTTVQAVASAIGRAIEDEARFGRIRDLEAKHFRKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGAMAGISPMFQPCVVPPKPWTGITGGGYWANGRRPLALVRTRSKKALMHYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIRPAIDSGKGLMFTQPNQAAGYMAKLIWESVNVTVVAAVEAMNWLKSAAKLLAAEVKDKKTGEILRKRCAVHWVTPDGFPVWQEYKKPIQTRLNLMFLGQFRLQPTINTNKDSEIDARKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 13 - protein, 1778 aa, synthetic construct
MFLEPPKKNRKVVASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVSELWKIELTIVKQLMGSEVSAKLAAFKTLLFDTPEQQTTKNMMTLINPDGEYLYEIEIEYTGKPESLTAADVIKIKNTVLTLISPNHLMLTAYHQAIEFIASHILSSEILLARIKSGKWGLKRLLPRVKSMTKAYYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFNIKAEMKPFISLTSADPNVLLKNFESIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINYVRWEIVKIREDRQQDLKTGGYFGNDFKTAELTWLNYMDPFSFEELAKGPSGMYFAGAKTGIYRAQTALISFIKQEIIQKISHQSWVIDLGIGKGQDLGRYLDAGVRHLVGIDKDQTALAELVYRKFSHATTRQHKHATNIYVLHQDLAEPAKEISEKVHQIYGFPKEGASSIVSNLFIHYLMKNTQQVENLAVLCHKLLQPGGMVWFTTMLGEQVLELLHENRIELNEVWEARENEVVKFAIKRLFKEDILQETGQEIGVLLPFSNGDFYNEYLVNTAFLIKIFKHHGFSLVQKQSFKDWIPEFQNFSKSLYKILTEADKTWTSLFGFICLRKNGGGGSGGGGSGGGGSLNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYTTIKTTLACLTSTDNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTRSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKFPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIRPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDARKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRGILESDFAFA

SEQ ID NO: 14 - protein, 1778 aa, synthetic construct
MFLEPPKKNRKVVASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVSELWKIELTIVKQLMGSEVSAKLAAFKTLLFDTPEQQTTKNMMTLINPDGEYLYEIEIEYTGKPESLTAADVIKIKNTVLTLISPNHLMLTAYHQAIEFIASHILSSEILLARIKSGKWGLKRLLPRVKSMTKAYYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFNIKAEMKPFISLTSADPNVLLKNFESIFKKKARPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINYVRWEIVKIREDRQQDLKTGGYFGNDFKTAELTWLNYMDPFSFEELAKGPSGMYFAGAKTGIYRAQTALISFIKQEIIQKISHQSWVIDLGIGKGQDLGRYLDAGVRHLVGIDKDQTALAELVYRKFSHATTRQHKHATNIYVLHQDLAEPAKEISEKVHQIYGFPKEGASSIVSNLFIHYLMKNTQQVENLAVMCHKLLQPGGMVWLTTMLGEQVLELLHENRIELNEVWEARENEVVKFAIKRLFKEDILQETGQEIGVLLPFSNGDFYNEYLVNTAFLIKIFKHHGFSLVQKQSFKDWIPEFQNFSKSLYKILTEADKTWTSLFGFICLRKNGGGGSGGGGSGGGGSLNTINIAKNDFSDIELAAIPFNTLADHYGERLAREQLALEHESYEMGEARFRKMFERQLKAGEVADNAAAKPLITTLLPKMIARINDRFEEVKAKRGKRPTAFQFLQEIKPEAVAYITIKTTLACLTSTDNTTVQAVASAIGRAIEDEARFGRIRDLEAKHFRKNVEEQLNKRVGHVYKKAFVQVVEADMLSKGLLGGEAWSSWHKEDSIHVGVRCIEMLIESTGMVSLHRQNAGVVCQDSETIELAPEYAEAIATRAGALAGISPMFQPCVVPPKPWTGITGGGYWANGRRPLALVRTRSKKALMHYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDARKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRGILESDFAFA

SEQ ID NO: 15 - protein, 1778 aa, synthetic construct
MFLEPPKKKKKVVASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVSELWKIELTIVKQLMGSEVSAKLAAFKTLLFDTPEQQTTKNMMTLINPDGEYLYEIEIEYTGKPESLTAADVIKIKNTVLTLISPNHLMLTAYHQAIEFIASHILSSEILLARIKSGKWGLKRLLPRVKSMTKANYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFDIKAEMKPFISLTSADPNVLLKNFESIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINYVRWEIVKIREDRQQDLKTGGYFGNDFKTAELTWLNYMDPFSFEELAKGPSGMYFAGAKTGIYRAQTALISFIKQEIIRKISHQSWVIDLGIGKGQDLGRYLDAGVRHLVGIDKDQTALAELVYRKFSHATTRQHKHATNIYVLHQDLAEPAKEISEKVHQIYGFPKEGASSIVSNLFIHYLMKNTQQVENLAVMCHKLLQPGGMVWLTTMLGEQVLELLHENRIELNEVWEARENEVVKFAIKRLFKEDILQETGQEIGVLLPFSNGDFYNEYLVNTAFLIKIFKHHGFSLVQKQSFKDWIPEFQNFSKSLYKILTEADKTWTSLFGFICLRKNGGGGSGGGGSGGGGSLNTINIAKNDFSDIELAAIPFNTLAGHYGERLAREQLALEHESYEMGEARFRKMFERQLKAGEVADNAAAKPLITTLLPKMIARINDWFEEVKAKRGKRPTAFQFLQEIKPEAVAYTTIKTTLACLTSTDNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLRRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKFPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVNVTVVAAVEAMNWLKSAAKLLAAEVKDKKTGEILRKRCAVHWVTPDGFPVWQEYKKPIQTRLNLMFLGQFRLQPTINTNKDSEIDARKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRGILESDFAFA

SEQ ID NO: 16 - protein, 1778 aa, synthetic construct
MFLEPPKKNRKVVASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVSELWKIELTIVKQLMGSEVSAKLAAFKTLLFDTPEQQTTKNMMTLINPDGEYLYEIEIEYTGKPESLTAADVIKIKNTVLTLISPNHLMLTAYHQAIEFIASHILSSEILLARIKSGKWGLKRLLPRVKSMTKAYYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFNIKAEMKPFISLTSADPNVLLKNFESIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINYVRWEIVKIREDRQQDLKTGGYFGNDFKTAELTWLNYMDPFSFEELAKGPSGMYFAGAKTGIYRAQTALISFIKQEIIQKISHQSWVIDLGIGKGQDLGRYLDAGVRHLVGIDKDQTALAELVYRKFSHATTRQHKHATNIYVLHQDLAEPAKEISEKVHQIYGFPKEGASSIVSNLFIHYLMKNTQQVENLAVMCHKLLQPGGMVWLTTMLGEQVLELLHENRIELNEVWEARENEVVKFAIKRLFKEDILQETGQEIGVLLPFSNGDFYNEYLVNTAFLIKIFKHHGFSLVQKQSFKDWIPEFQNFSKSLYKILTEADKTWTSLFGFICLRKNGGGGSGGGGSGGGGSLNTINIAKNDFSDIELAAIPFNTLADHYGERLAREQLALEHESYEMGEARFRKMFERQLKAGEVADNAAAKPLITTLLPKMIARINDRFEEVKAKRGKRPTAFQFLQEIKPEAVAYITIKTTLACLTSADNTTVQAVASAIGRAVEDEARFGRIRDLEAKHFRKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGAMAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFSYNMDWRGRVYAVSMFNPQGNDMTKGLLTLAKGKPIGKEGYYWLKIHGANCAGVDKVPFPERIKFIEENHENIMACAKFPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVNVTVVAAVEAMNWLKSAAKLLAAEVKDKKTGEILRKRCAVHWVTPDGFPVWQEYKKPIQTRLNLMFLGQFRLQPTINTNKDSEIDARKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRGILESDFAFA

SEQ ID NO: 17 - protein, 1778 aa, synthetic construct
MFLEPPKKNRKVVASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVSELWKIELTIVKQLMGSEVSAKLAAFKTLLFDTPEQQTTKNMMTLINPDGEYLYEIEIEYTGKPESLTAADVIKIKNTVLTLISPNHLMLTAYHQAIEFIASHILSSEILLARIKSGKWGLKRLLPRVKSMTKANYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFNIKAEMKPFISLTSADPNVLLKNFESIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINYVRWEIVKIREDRQQDLKTGGYFGNDFKTAELTWLNYMDPFSFEELAKGPSGMYFAGAKTGIYRAQTALISFIKQEIIQKISHQSWVIDLGIGKGQDLGRYLDAGVRHLVGIDKDQTALAELVYRKFSHATTRQHKHATNIYVLNQDLAEPAKEISEKVHQIYGFPKEGASSIVSNLFIHYLMKNTQQVENLAVLCHKLLQPGGMVWFTTMLGEQVLELLHENRIELNEVWEARENEVVKFAIKRLFKEDILQETGQEIGVLLPFSNGDFYNEYLVNTAFLIKIFEHHGFSLVQKQSFKDWIPEFQNFSKSLYKILTEADKTWTSLFGFICLRKNGGGGSGGGGSGGGGSLNTINIAKNDFSDIELAAIPFNTLAGHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFRKNVEEQLNKRVGHVYKKAFMQVVEADMLSKGLLGGEAWSSWHKEDSIHVGVRCIEMLIESTGMVSLHRQNAGVVCQDSETIELAPEYAEAIATRAGALAGISPMFQPCVVPPKPWTGITGGGYWANGRRPLALVRTRSKKALMHYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQGSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIRPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDARKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 18 - protein, 1778 aa, synthetic construct
MFLEPPKKNRKVVASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVSELWKIELTIVKQLMGSEVSAKLAAFKTLLFDTPEQQTTKNMMTLINPDGEYLYEIEIEYTGKPESLTAADVIKIKNTVLTLISPNHLMLTAYHQAIEFIASHILSSEILLARIKSGKWGLKRLLPRVKSMTKANYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFNIKAEMKPFISLTSADPNVLLKNFESIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINYVRWEIVKIREDRQQDLKTGGYFGNDFKTAELTWLNYMDPFSFEELAKGPSGMYFAGAKTGIYRAQTALISFIKQEIIQKISHQSWVIDLGIGKGQDLGRYLDAGVRHLVGIDKDQTALAELVYRKFSHATTRQHKHATNIYVLHQDLAEPAKEISEKVHQIYGFPKEGASSIVSNLFIHYLMKNTQQVENLAVLCHKLLQPGGMVWFTTMLGEQVLELLHENRIELNEVWEARENEVVKFAIKRLFKEDILQETGQEIGVLLPFSNGDFYNEYLVNTAFLIKIFKHHGFSLVQKQSFKDWIPEFQNFSKSLYKILTEADKTWTSLFGFICLRKNGGGGSGGGGSGGGGSLNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADKTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGAMAGISPMFQPCVVPPKPWTGITGGGYWANGRRPLALVRTRSKKALMHYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRRQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDARKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 19 - protein, 1778 aa, synthetic construct
MFLEPPKKKKKVVASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVSELWKIELTIVKQLMSSEVSAKLAAFKTLLFDTPEQQTTRNMMTLINPDGEYLYEIEIEYTGKPESLTAADVIKIKNTVLTLISPNHLMLTAYHQAIEFIASHILSSEILLARIKSGKWGLKRLLPRVKSMTKANYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFDIKAEMKPFISLTSADPNVLLKNFESIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINYVRWEIVRIREDRQQDLKTGGYFGNDFKTAELTWLNYMDPFSFEELAKGPSGMYFAGAKTGIYRAQTALISFIKQEIIRKISHQSWVIDLGIGKGQDLGRYLDAGVRHLVGIDKDQTALAELVYRKFSHATTRQHKHATNIYVLNQDLAEPAKEISEKVHQIYGFPKEGASSIVSNLFIHYLMKNTQQVENLAVLCHKLLQPGGMVWFTTMLGEQVLELLHENRIELNEVWEARENEVVKFAIKRLFKEDILQETGQEIGVLLPFSNGDFYNEYLVNTAFLIKIFEHHGFSLVQKQSFKDWIPEFQNFSKSLYKILTEADKTWTSLFGFICLRKNGGGGSGGGGSGGGGSLNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKDVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTRSKKALMHYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKFPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIRPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDARKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 20 - protein, 1778 aa, synthetic construct
MFLEPPKKKKKVVASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVSELWKIELTIVKQLMSSEVSAKLAAFKTLLFDTPEQQTTRNMMTLINPDGEYLYEIEIEYTGKPESLTAADVIKIKNTVLTLISPNHLMLTAYHQAIEFIASHILSSEILLARIKSGKWGLKRLLPRVKSMTKANYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFDIKAEMKPFISLTSADPNVLLKNFESIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINYVRWEIVKIREDRQQDLKTGGYFGNDFKTAELTWLNYMDPFSFEELAKGPSGMYFAGAKTGIYRAQTALISFIKQEIIRKISHQSWVIDLGIGKGQDLGRYLDAGVRHLVGIDKDQTALAELVYRKFSHATTRQHKHATNIYVLNQDLAEPAKEISEKVHQIYGFPKEGASSIVSNLFIHYLMKNTQQVENLAVLCHKLLQPGGMVWFTTMLGEQVLELLHENRIELNEVWEARENEVVKFAIKRLFKEDILQETGQEIGVLLPFSNGDFYNEYLVNTAFLIKIFEHHGFSLVQKQSFKDWIPEFQNFSKSLYKILTEADKTWTSLFGFICLRKNGGGGSGGGGSGGGGSLNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKDVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTRSKKALMHYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKFPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIRPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDARKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 21 - protein, 1778 aa, synthetic construct
MFLEPPKKNRKVVASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVTELWKIELTIVKQLMSSEVSAKLAAFKTLLFDTPEQQTTKNMMTLINPDGEYLYEIEIEYTGKPESLTAADVIKIKNTVLTLISPNHLMLTAYHQAIEFIASHILSSEILLARIKSGKWGLKRLLPRVKSMTKANYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFNIKAEMKPFISLTSADPNVLLKNFESIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPPDSPLAFLYYHPDTSSFSNIDGKVLEMRCLKREINYVRWEIVRIREDRQQDLKTGGYFGNDFKTAELTWLNYMDPFSFEELAKGPSGMYFAGAKTGIYRAQTALISFIKQEIIQKISHQSWVIDLGIGKGQDLGRYLDAGVRHLVGIDKDQTALAELVYRKFSHATTRQHKHATNIYVLHQDLAEPAKEISEKVHQIYGFPKEGASSIVSNLFIHYLMKNTQQVENLAVLCHKLLQPGGMVWFTTMLGEQVLELLHENRIELNEVWEARENEVVKFAIKRLFKEDILQETGQEIGVLLPFSNGDFYNEYLVNTAFLIKIFKHHGFSLVQKQSFKDWIPEFQNFSKSLYKILTEADKTWTSLFGFICLRKNGGGGSGGGGSGGGGSLNTINIAKNDFSDIELAAIPFNTLADHYGERLAREQLALEHESYEMGEARFRKMFERQLKVGEVADNAAAKPLITTLLPKMIARINDWFEEVKAKRGKRPTAFQFLQEIKPEAVAYITIKTTLACLTSADKTTVQAVASAIGRAIEDEARFGRIRDLEAKHFRKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTRSKKALMHYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIRPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDARKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRGILESDFAFA

SEQ ID NO: 22 - protein, 1778 aa, synthetic construct
MFLEPPKKNRKVVASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVSELWKIELTIVKQLMGSEVSAKLAAFKTLLFDTPEQQTTKNMMTLINPDGEYLYEIEIEYTGKPESLTAADVIKIKNTVLTLISPNHLMLTAYHQAIEFIASHILSSEILLARIKSGKWGLKRLLPRVKSMTKANYMKFYPPVGYYVTDKADGIRGIAVIQDTQIYVVADQLYSLGTTGIEPLKPTILDGEFMPEKKEFYGFDVIMCEGNLLTQQGFETRIESLSKGIKVLQAFNIKAEMKPFISLTSADPNVLLKNFESIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINYVRWEIVKIREDRQQDLKTGGYFGNDFKTAELTWLNYMDPFSFEELAKGPSGMYFAGAKTGIYRAQTALISFIKQEIIRKISHQSWVIDLGIGKGQDLGRYLDAGVRHLVGIDKDQTALAELVYRKFSHATTRQHKHATNIYVLHQDLAEPAKEISEKVHQIYGFPKEGASSIVSNLFIHYLMKNTQQVENLAVMCHKLLQPGGMVWFTTMLGEQVLELLHENRIELNEVWEARENEVVKFAIKRLFKEDILQETGQEIGVLLPFSNGDFYNEYLVNTAFLIKIFKHHGFSLVQKQSFKDWIPEFQNFSKSLYKILTEADKTWTSLFGFICLRKNGGGGSGGGGSGGGGSLNTINIAKNDFSDIELAAIPFNTLADHYGERLAREQLALEHESYEMGEARFRKMFERQLKAGEVADNAAAKPLITTLLPKMIARINNWFEEVKAKRGKRPTAFQFLQEIKPEAVAYITIKTTLACLTSADKTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGAMAGISPMFQPCVVPPKPWTGITGGGYWANGRRPLALVRTHSKKALMRYEDVYMPEVYKAINIAQNTAWKINEKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVNVTVVAAVEAMNWLKSAAKLLAAEVKDKKTGEILRKRCAVHWVTPDGFPVWQEYKKPIQTRLNLMFLGQFRLQPTINTNKDSEIDARKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 23 - protein, 1778 aa, synthetic construct
MFLEPPKKNRKVVASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVSELWKIELTIVKQLMGSEVSAKLAAFKTLLFDTPEQQTTKNMMTLINPDGEYLYEIEIEYTGKPESLTAADVIKIKNTVLTLISPNHLMLTAYHQAIEFIASHILSSEILLARIKSGKWGLKRLLPRVKSMTKADYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFNIKAEMKPFISLTSADPNVLLKNFESIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINYVRWEIVKIREDRQQDLKTGGYFGNDFKTAELTWLNYMDPFSFEELAKGPSGTYFAGAKTGIYRAQTALISFIKQEIIQKISHQSWVIDLGIGKGQDLGRYLDAGVRHLVGIDKDQTALAELVYRKFSHATTRQHKHATNIYVLHQDLAEPAKEISEKVHQIYGFPKEGASSIVSNLFIHYLMKNTQQVENLAVLCHKLLQPGGMVWFTTMLGEQVLELLHENRIELNEVWEARENEVVKFAIKRLFKEDILQETGQEIGVLLPFSNGDFYNEYLVNTAFLIKIFKHHGFSLVQKQSFKDWIPEFQNFSKSLYKILTEADKTWTSLFGFICLRKNGGGGSGGGGSGGGGSLNTINIAKNDFSDIELAAIPFNTLADHYGERLAREQLALEHESYEMGEARFRKMFERQLKAGEVADNAAAKPLITTLLPKMIARINNWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWPSWHKEDSIHVGVRCIEMLIESTGMVSLHRQNAGVVCQDSETIELAPEYAEAIATRAGALAGISPMFQPCVVPPKPWTGITGGGYWANGRRPLALVRTRSKKALMHYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVNVTVVAAVEAMNWLKSAAKLLAAEVKDKKTGEILRKRCAVHWVTPDGFPVWQEYKKPIQTRLNLMFLGQFRLQPTINTNKDSEIDARKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 24 - protein, 1773 aa, synthetic construct
MFLEPPKKKIKVVASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVSELWKIELTIVKQLMGSEVSAKLAAFKTLLFDTPEQQTTKNMMTLINPDGEYLYEIEIEYTGKPESLTAADVIKIKNTVLTLISPNHLMLTAYHQAIEFIASHILSSEILLARIKSGKWGLKRLLPRVKSMTKANYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFDIKAEMKPFISLTSADPNVLLKNFESIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINYVRWEIVKIREDRQQDLKTGGYFGNDFKTAELTWLNYMDPFSFEELAKGPSGMYFAGAKTGIYRAQTALISFIKQEIIQKISHQSWVIDLGIGKGQDLGRYLDAGVRHLVGIDKDQTALAELVYRKFSHATTRQHKHATNIYVLHQDLAEPAKEISEKVHQIYGFPKEGASSIVSNLFIHYLMKNTQQVENLAVMCHKLLQPGGMVWLTTMLGEQVLELLHENRIELNEVWEARENEVVKFAIKRLFKEDILQETGQEIGVLLPFSNGDFYNEYLVNTAFLIKIFKHHGFSLVQKQSFKDWIPEFQNFSKSLYKILTEADKTWTSLFGFICLRKNGGGGSGGGGSLNTINIAKNDFSDIELAAIPFNTLADHYGERLAREQLALEHESYEMGEARFRKMFERQLKAGEVADNAAAKPLITTLLPKMIARINDRFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGAMAGISPMFQPCVVPPKPWTGITGGGYWANGRRPLALVRTRSKKALMHYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRRQVLEDTIQPAIDSGKGLMFTQPNQAAGYMAKLIWESVNVTVVAAVEAMNWLKSAAKLLAAEVKDKKTGEILRKRCAVHWVTPDGFPVWQEYKKPIQTRLNLMFLGQFRLQPTINTNKDSEIDARKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 25 - DNA, 17 bp, synthetic construct
  source 1..17: mol_type = other DNA
taatacgactcactata

SEQ ID NO: 26 - DNA, 17 bp, synthetic construct
  source 1..17: mol_type = other DNA
taatacgactcactaaa

SEQ ID NO: 27 - DNA, 17 bp, synthetic construct
  source 1..17: mol_type = other DNA
taatacgactcactgta

SEQ ID NO: 28 - DNA, 17 bp, synthetic construct
  source 1..17: mol_type = other DNA
taatacgactcactcta

SEQ ID NO: 29 - DNA, 17 bp, synthetic construct
  source 1..17: mol_type = other DNA
taataccggtcactata

SEQ ID NO: 30 - DNA, 17 bp, synthetic construct
  source 1..17: mol_type = other DNA
taatacctgacactata

SEQ ID NO: 31 - DNA, 17 bp, synthetic construct
  source 1..17: mol_type = other DNA
taataaccctcactata

SEQ ID NO: 32 - DNA, 17 bp, synthetic construct
  source 1..17: mol_type = other DNA
taataactatcactata

SEQ ID NO: 33 - DNA, 17 bp, synthetic construct
  source 1..17: mol_type = other DNA
taataacccacactata

SEQ ID NO: 34 - protein, 1833 aa, synthetic construct
MGHHHHHHHHHHGSSAWSHPQFEKGGGSGGGSGGGSWSHPQFEKGSLEVLFQGPGSFFEPPKKKRKVVASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVSELWKIELTIVKQLMGSEVSAKLAAFKTLLFDTPEQQTTKNMMTLINPDGEYLYEIEIEYTGKPESLTAADVIKIKNTVLTLISPNHLMLTAYHQAIEFIASHILSSEILLARIKSGKWGLKRLLPRVKSMTKADYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFNIKAEMKPFISLTSADPNVLLKNFESIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINYVRWEIVKIREDRQQDLKTGGYFGNDFKTAELTWLNYMDPFSFEELAKGPSGMYFAGAKTGIYRAQTALISFIKQEIIQKISHQSWVIDLGIGKGQDLGRYLDAGVRHLVGIDKDQTALAELVYRKFSHATTRQHKHATNIYVLHQDLAEPAKEISEKVHQIYGFPKEGASSIVSNLFIHYLMKNTQQVENLAVLCHKLLQPGGMVWFTTMLGEQVLELLHENRIELNEVWEARENEVVKFAIKRLFKEDILQETGQEIGVLLPFSNGDFYNEYLVNTAFLIKIFKHHGFSLVQKQSFKDWIPEFQNFSKSLYKILTEADKTWTSLFGFICLRKNGGGGSGGGGSGGGGSLNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA